SEQUENCE LISTING (70 sequences)

SEQ ID NO: 1 - DNA, 3112 bp, Zea mays
caaccctgccttctcttcttccttattcccagtttcccacagcttgcgcggtctcactctctctcggcgacgactacgccaaggcccaagtgtgccgaggtattttttaggctcaggtcgctaccgccctacggccccggtcttcttccggcggcccttcgccggcgacgagaaccagccggtaggtcttcctcacctcgctgctgtgcgaaagcgagcacacaacaaaccctaacttactatttgtattcggatctgatctactgggtccgcccctgatttgaggcagtattcctcatatttggcaaaggaggaattccagccagattctctattctccccctgatttccagcagcttccaaaactttctaaagaaacaaaagaaaagtctaacacagctcgtaatccttccgccagggcagcagacggatggcttcgtcggtgccggcgccatcggggtcggtgatcaccgtggcgtcgtcttcttcctcagcagccgcggccgcggtgtgcggcacgggctccccatgcgctgcgtgcaagttcctgcgtcgcaagtgccagccggactgcgtgttcgcgccctacttcccaccggacaacccgcagaagttcgtgcacgtgcaccgcgtcttcggcgcgagcaacgtgaccaagctgctgaacgagctccaccccttccagcgcgaggacgccgtgaactccctcgcctacgaggccgacatgcgcctccgcgaccccgtctacggctgcgtcggcgtcatctccatcctccagcacaacctactattagacagatatattccagattgtaatcttgatttctttccaactaaccggacgtagatagcacaggatctccttgatttttggcgctgatatcgcttgtgatacggcagcctctgttagcctccggttagagtttggtacgatgtaacaacctccgctgctttgtcagcctataaatacatatacgcggctgctcaagggcagtagagaacgcttcctaattccttcacctacgacagctccagcaggacctcgcccgcgccaagtacgaactctccaagtaccaggtgacgtgacgatgttcgtcgatcgacatatatatatcgtcgtctcacaacgtaccattcgtcgctggctcgcacacgttgtagagatcacatgagcgaacgtgtgttgcgtcggctgttaattcgtcctcgctcttcttccttgtaggcggcagcggcagcttctgcgtccacggcgccgaccgggccgcaagcgatggcagagttcatcggcaacgcgatgccgaacggcgcgcacaacttcatcaacattggacactcggcggcgctcgggtccctcggaggctccgcctccgtgttcgggcaggagcagttcggcaacgcgcagatgctgtccaggagctacgacggcggcgagcccatcgcgaggctggggatcaacggcggcggctacgagttcggctactcgaccgcgatgggcgggtctggcgctgtctccggcctcgggacgctcggcatcagcccgttcttgaagtctggtaccgccggcggcgacgagaagcccaacggcggtcagtaggcggatcgatccatccactcccatcaatcttgtcaggtttggtttgttttggttttgcatgctgcctgcacgcgccgtgttttattttgccttccctttttctgattaattcgcggagtttggtggactacggttagtcgatttattttctaggtagaggaacaattatgcatgactaatataattaagggtttcattccatggctaaatataccatttgcatgatgctagttggagctaacgtccttgttcaaacaaagcatcttagaactgtttggatctacatattaaattgatggctaggtgcatgcatatgtacttgaacagcagggcttttccgaacttctgatcagcagaatccagcatctgcttttgtcaaactgttttgcttttagatcgtgggtacttgttagtatccatgcacagcacacggtgtggccgtgtgtggtgaacaaatctattttcagaattgcacttagctcatcatgtttggtgtacctaattaagcttccgtttctttctgtattctttctttttttataaggacgcttctgttaaatcggttgaaacagaagcaatatcattggttattagcaaatcctgaagaatgcatgcaagttcttctttatctgtatcactatttcttagcacagttctctaactttagatgcacctacaaatctattttttttataaaaaatttgtctatatatgagagcacacagaatttttggacttgtgcatcaataatcacatgctcttatgcctgaatgcctgatgtccagtggattagtgttcgatctcatgaattcgagctgtcctccattcctcctgcgtgaaaaatatactagaaatgctcctatatagtcctatatatcaagaacacgcacgggtatgttctccgtgaaatatcagtgtatcagtaaccgagcatcatatcaccaactagctagcgacagcagtagctactataatagggtgtagctactataatagcctgttggggtcgagaaatgagatagtttatcatctttttactcatcactttattgtttagttcgtggaaatgaatgggttaatctatcatcgttttattactcatatgttaataattcataataatgaggaatacgttcgtttcactaaatttatgtaataaattcatgatatatcatcatatatagaacagattgactcttcaaaccaaacactccatacgtatctgtaggaacatttctttctttctttaacttaaaactggttcgagaaaagagaactgcccatttcattagtttataaagttaacagcgcctgcagagaaaaagcatatggccggaatgtgctgtcagctttgaactgtgctaataatatataagtacccccaacacacagaagcttctgtgttgaaatagtatagctgtattccattctttgtctattcataagtgggggaaaaaacgtacctgcatttagcatatccagtttctatttacgacagtttgtgaacagctcata

SEQ ID NO: 2 - DNA, 780 bp, Artificial Sequence
  cDNA of haploid induction conferring ig allele - transcript 1
atggcttcgtcggtgccggcgccatcggggtcggtgatcaccgtggcgtcgtcttcttcctcagcagccgcggccgcggtgtgcggcacgggctccccatgcgctgcgtgcaagttcctgcgtcgcaagtgccagccggactgcgtgttcgcgccctacttcccaccggacaacccgcagaagttcgtgcacgtgcaccgcgtcttcggcgcgagcaacgtgaccaagctgctgaacgagctccaccccttccagcgcgaggacgccgtgaactccctcgcctacgaggccgacatgcgcctccgcgaccccgtctacggctgcgtcggcgtcatctccatcctccagcacaacctactattactccagcaggacctcgcccgcgccaagtacgaactctccaagtaccaggcggcagcggcagcttctgcgtccacggcgccgaccgggccgcaagcgatggcagagttcatcggcaacgcgatgccgaacggcgcgcacaacttcatcaacattggacactcggcggcgctcgggtccctcggaggctccgcctccgtgttcgggcaggagcagttcggcaacgcgcagatgctgtccaggagctacgacggcggcgagcccatcgcgaggctggggatcaacggcggcggctacgagttcggctactcgaccgcgatgggcgggtctggcgctgtctccggcctcgggacgctcggcatcagcccgttcttgaagtctggtaccgccggcggcgacgagaagcccaacggcggtcag

SEQ ID NO: 3 - DNA, 777 bp, Artificial Sequence
  cDNA of haploid induction conferring ig allele - transcript 2
atggcttcgtcggtgccggcgccatcggggtcggtgatcaccgtggcgtcgtcttcttcctcagcagccgcggccgcggtgtgcggcacgggctccccatgcgctgcgtgcaagttcctgcgtcgcaagtgccagccggactgcgtgttcgcgccctacttcccaccggacaacccgcagaagttcgtgcacgtgcaccgcgtcttcggcgcgagcaacgtgaccaagctgctgaacgagctccaccccttccagcgcgaggacgccgtgaactccctcgcctacgaggccgacatgcgcctccgcgaccccgtctacggctgcgtcggcgtcatctccatcctccagcacaacctactattactccagcaggacctcgcccgcgccaagtacgaactctccaagtaccaggcggcagcggcagcttctgcgtccacggcgccgaccgggccgcaagcgatggcagagttcatcggcaacgcgatgccgaacggcgcgcacaacttcatcaacattggacactcggcggcgctcgggtccctcggaggctccgcctccgtgttcgggcaggagcagttcggcaacgcgcagatgctgtccaggagctacgacggcggcgagcccatcgcgaggctggggatcaacggcggcggctacgagttcggctactcgaccgcgatgggcgggtctggcgctgtctccggcctcgggacgctcggcatcagcccgttcttgaagtctggtaccgccggcggcgacgagaagcccaacggcggt

SEQ ID NO: 4 - protein, 260 aa, Zea mays
MASSVPAPSGSVITVASSSSSAAAAAVCGTGSPCAACKFLRRKCQPDCVFAPYFPPDNPQKFVHVHRVFGASNVTKLLNELHPFQREDAVNSLAYEADMRLRDPVYGCVGVISILQHNLLLLQQDLARAKYELSKYQAAAAASASTAPTGPQAMAEFIGNAMPNGAHNFINIGHSAALGSLGGSASVFGQEQFGNAQMLSRSYDGGEPIARLGINGGGYEFGYSTAMGGSGAVSGLGTLGISPFLKSGTAGGDEKPNGGQ

SEQ ID NO: 5 - protein, 259 aa, Zea mays
MASSVPAPSGSVITVASSSSSAAAAAVCGTGSPCAACKFLRRKCQPDCVFAPYFPPDNPQKFVHVHRVFGASNVTKLLNELHPFQREDAVNSLAYEADMRLRDPVYGCVGVISILQHNLLLLQQDLARAKYELSKYQAAAAASASTAPTGPQAMAEFIGNAMPNGAHNFINIGHSAALGSLGGSASVFGQEQFGNAQMLSRSYDGGEPIARLGINGGGYEFGYSTAMGGSGAVSGLGTLGISPFLKSGTAGGDEKPNGG

SEQ ID NO: 6 - DNA, 2839 bp, Zea mays
caaccctgccttctcttcttccttattcccagtttcccacagcttgcgcggtctcactctctctcggcgacgactacgccaaggcccaagtgtgccgaggtattttttaggctcaggtcgctaccgccctacggccccggtcttcttccggcccttcgccggcgacgagaaccagcaggtaggtcttcctcacctcgctgctgtgcgaaagcgagcacacaacaaaccctaacttactatttgtattcggatctgatctactggatccgcccctgatttgaggcagtattcctcatatttggcaaaggaggccgaattccagccagattctctattctccccctgatttccagcagcttccaaaactttctaaagaaacaaaagaagagtctaacacagctcgtgatccttccgccagggcagcagacggatggcttcgtcggtgccggcgccatcggggtcggtgatcaccgtggcgtcgtcttcttcctcagcagccgcggccgcggtgtgcggcacgggctccccatgcgctgcgtgcaagttcctgcgtcgcaagtgccagccggactgcgtgttcgcgccctacttcccaccggacaacccgcagaagttcgtgcacgtgcaccgcgtcttcggcgcgagcaacgtgaccaagctgctgaacgagctccaccccttccagcgcgaggacgccgtgaactccctcgcctacgaggccgacatgcgcctccgcgaccccgtctacggctgcgtcggcgtcatctccatcctccagcacaacctacgacagctccagcaggacctcgcccgcgccaagtacgaactctccaagtaccaggtgatgtgacgatcgtcgatcgacatatatatatatatcgtcgtctcacaacgtaccattcgctggctcgcacatgttgtagagatcacatgagcgaacgtgtgttgcgtcggctgttaattcgtcctcgctcttcttccttgtaggcggcagcggcagcttctgcgtccacggcgccgacagggccgcaagcgatggcagagttcatcggcaacgcgatgccgaacggcgcgcacaacttcatcaacattggacactcggcggcgctcgggtccctcggaggctccgcctccgtgttcgggcaggagcagttcggcaacgcgcagatgctgtccaggagctatgacggcggcgagcccatcgcgaggctggggatcaacggcggcggctacgagttcggctactcgaccgcgatgggcgggtctggcgctgtctccggcctcgggacgctcggcatcagcccgttcttgaagtctggtaccgccggcggcgacgagaagcccaacggcggtcagtaggcggatcgatccatccactcccatcaatcttgtcaggtttggtttgttttggttttgcatgctgcctgcacgcgcggtgttttattttgccttccctttttctgattaattcgcggagtttggtggactacggttagtcggtttattttctaggtagaggaacaattatgcatgactaataataattaagggtttcatttcatggttaaatataccatttgcatgatgctagttggagctaacgtccttgttcaaacaaagtatcgtagaattgtttggatctacatattaaattgatggctaggtgcatgcatatgtacttgaacagcagggcttttccgaacttctgatcagcagaatccagcatctgcttttgtcaaactgttttgcttttagatcgagggtacttgttagtatccatgcacagcacacggtgtggccgtatggtgaacaaatctattttcagatttgcgctcagctcatcgtgtgtggtgtacctaattaagcttccgtttttttgtattctttcttttttataaggacgctcctgttaaatcgtttgaaacagaagcaacatcattgattattagcaaatcctgaagaatgcatgcaagttcttctttatctgtatcactatctcttagcaccgttctctaactttagatgcacgtacaagtctaatttttttttataaaaaaattgtctatatataggagcacacagaatttttggacgtgtacatcaatcatcacatgctcttatgcctgtggattggtgttcgactagtgaattcgagctgtcctccatattttccatactagaaatgctcctatatagtcctatatatcaagaacacgcacgggtgtgttctccgtgaaatatcagtgtatcagtaaccgggcatcatatcaccaactagctagcgacagcagtagctactacaatagggtgtagctactataatagcgtgttgggttcgagaaataagatagttcattatttttttactcctcattttattgtttagttcgtggaaatgaatggattgatccatcatcattttattactcatatgttaataattcataataatgaggaatacgtttgttccactaaatttatgtaataaactcatgatgcatcatcatatatagaacggattgactcttcaaaccaaacactctctacgtatctctaggaacatttctttctttctttaacttaaaactggttcgagaaaagagaactgcaatttcattagtttgtaaagttaacagagcttgcagagaaaaaacatatgtccggaatgtgctgtcagctttgaactgtgctaataatataagtaccccaacacacagaagcttctgtgttgaaatagtatagctgtattccattctttgtctattcataagtgggaaaaaaacgtacctgcatttagcatatgcagtttctatttacgacagtttgtgaacagctcata

SEQ ID NO: 7 - DNA, 780 bp, Artificial Sequence
  cDNA of B73 ig allele - transcript 1
atggcttcgtcggtgccggcgccatcggggtcggtgatcaccgtggcgtcgtcttcttcctcagcagccgcggccgcggtgtgcggcacgggctccccatgcgctgcgtgcaagttcctgcgtcgcaagtgccagccggactgcgtgttcgcgccctacttcccaccggacaacccgcagaagttcgtgcacgtgcaccgcgtcttcggcgcgagcaacgtgaccaagctgctgaacgagctccaccccttccagcgcgaggacgccgtgaactccctcgcctacgaggccgacatgcgcctccgcgaccccgtctacggctgcgtcggcgtcatctccatcctccagcacaacctacgacagctccagcaggacctcgcccgcgccaagtacgaactctccaagtaccaggcggcagcggcagcttctgcgtccacggcgccgacagggccgcaagcgatggcagagttcatcggcaacgcgatgccgaacggcgcgcacaacttcatcaacattggacactcggcggcgctcgggtccctcggaggctccgcctccgtgttcgggcaggagcagttcggcaacgcgcagatgctgtccaggagctatgacggcggcgagcccatcgcgaggctggggatcaacggcggcggctacgagttcggctactcgaccgcgatgggcgggtctggcgctgtctccggcctcgggacgctcggcatcagcccgttcttgaagtctggtaccgccggcggcgacgagaagcccaacggcggtcag

SEQ ID NO: 8 - DNA, 777 bp, Artificial Sequence
  cDNA of B73 ig allele - transcript 2
atggcttcgtcggtgccggcgccatcggggtcggtgatcaccgtggcgtcgtcttcttcctcagcagccgcggccgcggtgtgcggcacgggctccccatgcgctgcgtgcaagttcctgcgtcgcaagtgccagccggactgcgtgttcgcgccctacttcccaccggacaacccgcagaagttcgtgcacgtgcaccgcgtcttcggcgcgagcaacgtgaccaagctgctgaacgagctccaccccttccagcgcgaggacgccgtgaactccctcgcctacgaggccgacatgcgcctccgcgaccccgtctacggctgcgtcggcgtcatctccatcctccagcacaacctacgacagctccagcaggacctcgcccgcgccaagtacgaactctccaagtaccaggcggcagcggcagcttctgcgtccacggcgccgacagggccgcaagcgatggcagagttcatcggcaacgcgatgccgaacggcgcgcacaacttcatcaacattggacactcggcggcgctcgggtccctcggaggctccgcctccgtgttcgggcaggagcagttcggcaacgcgcagatgctgtccaggagctatgacggcggcgagcccatcgcgaggctggggatcaacggcggcggctacgagttcggctactcgaccgcgatgggcgggtctggcgctgtctccggcctcgggacgctcggcatcagcccgttcttgaagtctggtaccgccggcggcgacgagaagcccaacggcggt

SEQ ID NO: 9 - protein, 260 aa, Zea mays
MASSVPAPSGSVITVASSSSSAAAAAVCGTGSPCAACKFLRRKCQPDCVFAPYFPPDNPQKFVHVHRVFGASNVTKLLNELHPFQREDAVNSLAYEADMRLRDPVYGCVGVISILQHNLRQLQQDLARAKYELSKYQAAAAASASTAPTGPQAMAEFIGNAMPNGAHNFINIGHSAALGSLGGSASVFGQEQFGNAQMLSRSYDGGEPIARLGINGGGYEFGYSTAMGGSGAVSGLGTLGISPFLKSGTAGGDEKPNGGY

SEQ ID NO: 10 - protein, 260 aa, Zea mays
MASSVPAPSGSVITVASSSSSAAAAAVCGTGSPCAACKFLRRKCQPDCVFAPYFPPDNPQKFVHVHRVFGASNVTKLLNELHPFQREDAVNSLAYEADMRLRDPVYGCVGVISILQHNLRQLQQDLARAKYELSKYQAAAAASASTAPTGPQAMAEFIGNAMPNGAHNFINIGHSAALGSLGGSASVFGQEQFGNAQMLSRSYDGGEPIARLGINGGGYEFGYSTAMGGSGAVSGLGTLGISPFLKSGTAGGDEKPNGGQ

SEQ ID NO: 11 - DNA, 537 bp, Artificial Sequence
  cDNA of wildtype CENH3 protein derived from Arabidopsis thaliana
atggcgagaaccaagcatcgcgttaccaggtcacaacctcggaatcaaactgatgccgccggtgcttcatcttctcaggcggcaggtccaactacgaccccgacaaggagaggcggtgaaggtggagataatactcaacaaacaaatcctacaacttcaccagctactggtacaaggagaggggctaagagatccagacaggctatgccacgaggctcacagaagaagtcttatcgatacaggccaggaaccgttgctctaaaagagattcgccatttccagaagcagacaaaccttcttattccggctgccagtttcataagagaagtgagaagtataacccatatgttggcccctccccaaatcaatcgttggacagctgaagctcttgttgctcttcaagaggcggcagaagattacttggttggtttgttctcagattcaatgctctgtgctatccatgcaagacgtgttactctaatgagaaaagactttgaacttgcacgccggcttggaggaaaaggcagaccatggtga

SEQ ID NO: 12 - protein, 178 aa, Arabidopsis thaliana
MARTKHRVTRSQPRNQTDAAGASSSQAAGPTTTPTRRGGEGGDNTQQTNPTTSPATGTRRGAKRSRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 13 - DNA, 471 bp, Artificial Sequence
  cDNA of wildtype CENH3 protein derived from Zea mays
atggctcgaaccaagcaccaggccgtgaggaagacggcggagaagcccaagaagaagctccagttcgagcgctcaggtggtgcgagtacctcggcgacgccggaaagggctgctgggaccgggggaagagcggcgtctggaggtgactcagttaagaagacgaaaccacgccaccgctggcggccagggactgtagcgctgcgggagatcaggaagtaccagaagtccactgaaccgctcatcccctttgcgcctttcgtccgtgtggtgagggagttaaccaatttcgtaacaaacgggaaagtagagcgctataccgcagaagccctccttgcgctgcaagaggcagcagaattccacttgatagaactgtttgaaatggcgaatctgtgtgccatccatgccaagcgtgtcacaatcatgcaaaaggacatacaacttgcaaggcgtatcggaggaaggcgttgggca

SEQ ID NO: 14 - protein, 157 aa, Zea mays
MARTKHQAVRKTAEKPKKKLQFERSGGASTSATPERAAGTGGRAASGGDSVKKTKPRHRWRPGTVALREIRKYQKSTEPLIPFAPFVRVVRELTNFVTNGKVERYTAEALLALQEAAEFHLIELFEMANLCAIHAKRVTIMQKDIQLARRIGGRRWA

SEQ ID NO: 15 - DNA, 540 bp, Artificial Sequence
  cDNA of wildtype CENH3 protein derived from Brassica napus
atggcgagaaccaaacatttcgcttccagggcacgagatcgcaatcgaactaatgcgactgcttcatcttcggcggcggcggcggaaggtccgagtgcgaccccgacgagaagagaaggcagccaaggagaagctcaacagacaactcctactacgactccaccagccggtagaaaaaaaggagggactaagcgaactaaacaagctatgcctaaaagttccaacaagaagaagacattccgttacaagcctggaaccgttgccctcagagagattcgccatttccagaagaccaccaaacttcttatccctgccgctagtttcatccgagaagtgagaagtgtcacccagatctttgcccctcccgatgttacccgttggactgctgaagctcttatggctattcaagaggcggctgaagattttttaattggcttgttctctgatgctatgctttgcgctatccatgcaaggcgtgttactctaatgagaaaagattttgagcttgcacgccgtcttggaggaaaaggcagaccattg

SEQ ID NO: 16 - protein, 180 aa, Brassica napus
MARTKHFASRARDRNRTNATASSSAAAAEGPSATPTRREGSQGEAQQTTPTTTPPAGRKKGGTKRTKQAMPKSSNKKKTFRYKPGTVALREIRHFQKTTKLLIPAASFIREVRSVTQIFAPPDVTRWTAEALMAIQEAAEDFLIGLFSDAMLCAIHARRVTLMRKDFELARRLGGKGRPL

SEQ ID NO: 17 - DNA, 471 bp, Artificial Sequence
  cDNA of wildtype CENH3 protein derived from Sorghum bicolor
atggctcgaaccaagcaccaggccgtgaggaagctgccgcagaagcccaagaagaagctccagttcgagcgcgcaggtggggcgagtacgtcggcgaccccggagaggaggaatgctgggaccgggggaggagccgcggctcgcgttgcacgggggcgtgtggagaagaagcatcgctggcgggcagggactgtagcgctgcgggagatcaggaagtaccagaagtccactgagccgctcatcccctttgcgcccttcgtacgtgtggtcaaagagttaactgcattcataacagactggaggatagggcgctacacccctgaagccctccttgcgctgcaagaggcagcagaattccacttgatagaactgtttgaagtggcgaatctgtgtgccatccatgccaagcgcgtaacagtcatgcaaaaggacatacaacttgcaaggcgtatcggaggaaggcgttggtcg

SEQ ID NO: 18 - protein, 157 aa, Sorghum bicolor
MARTKHQAVRKLPQKPKKKLQFERAGGASTSATPERRNAGTGGGAAARVARGRVEKKHRWRAGTVALREIRKYQKSTEPLIPFAPFVRVVKELTAFITDWRIGRYTPEALLALQEAAEFHLIELFEVANLCAIHAKRVTVMQKDIQLARRIGGRRWS

SEQ ID NO: 19 - DNA, 471 bp, Artificial Sequence
  cDNA of mutant CENH3 protein (E35K) derived from Zea mays
atggctcgaaccaagcaccaggccgtgaggaagacggcggagaagcccaagaagaagctccagttcgagcgctcaggtggtgcgagtacctcggcgacgccgaaaagggctgctgggaccgggggaagagcggcgtctggaggtgactcagttaagaagacgaaaccacgccaccgctggcggccagggactgtagcgctgcgggagatcaggaagtaccagaagtccactgaaccgctcatcccctttgcgcctttcgtccgtgtggtgagggagttaaccaatttcgtaacaaacgggaaagtagagcgctataccgcagaagccctccttgcgctgcaagaggcagcagaattccacttgatagaactgtttgaaatggcgaatctgtgtgccatccatgccaagcgtgtcacaatcatgcaaaaggacatacaacttgcaaggcgtatcggaggaaggcgttgggca

SEQ ID NO: 20 - protein, 157 aa, Artificial Sequence
  mutant CENH3 protein (E35K)
MARTKHQAVRKTAEKPKKKLQFERSGGASTSATPKRAAGTGGRAASGGDSVKKTKPRHRWRPGTVALREIRKYQKSTEPLIPFAPFVRVVRELTNFVTNGKVERYTAEALLALQEAAEFHLIELFEMANLCAIHAKRVTIMQKDIQLARRIGGRRWA

SEQ ID NO: 21 - DNA, 3293 bp, Sorghum bicolor
cagccttgagcccatgtaaatggtcctacacaaggccccggtcagcaagggccttttcttcttcctttgcccccccagattgcgcgctctctccctcagagcctcacaactcggcgacgactacgccaagtgcccaggtcttctccaggcccggtcgctgctgctctacaccagtttcccgggtcttcttcggagttcggactcgtcttcgccggcgacgagcaccgccaggtacgcccaccctttggtcatcccttccttctgttctgtggaaaccgagcacccaaaccctaacttgctatttgtattcggatctgatctactgggtccgcccctgatttattttgggcagtattcctcatttttggcaaaagaggaagtccagccagattctctattctcccccctgatttccagcagcttctaaaactttctaaacaaactagaaacaagagtctaacacacagcttgtgatcctttgtgccagggcagcagacggatggcttcgtcggtgccggcgccatcggggtcggtgatcaccgtggcatcgtcttcttcctcagcagccgcggccgcggtgtgtggcacgggctcgccatgcgctgcgtgcaagttcctgcgccgcaagtgccagccggactgcgtgttcgcaccctacttcccaccggacaacccgcagaagttcgtgcacgtgcaccgtgtcttcggcgccagcaacgtgaccaagctgctgaacgagctccaccctttccagcgcgaggacgccgtgaactcgctcgcctacgaggccgacatgcgcctccgcgaccccgtctacggctgcgttggcgtcatctccatcctccagcacaacctacgccagctccagcaggacctcgcccgcgccaagtacgagctctccaagtaccaggtgatgtacatgacgatatcgtcgtcgacatatcatccgatccatgtcaccgtaccacatgttgtagagatcacacatgagcgaacgtgtgttgtgtcgcgtcggcggctaattccctgctcgatcttcgtcgttcttccttgtgtatataggcggcagcggcagcttctgcgtccacggcggcgcccaacgggccgcaggcgatggcagagttcatcggcaacgcgatgccgaacggcgcgcacaacttcatcaacattggacactcggcggcgctcggctccctcggaggctccgccaccgtgttcggccaggagcagttcgccaacgcgcagatgctgtccaggagctacgacggcgagcccatcgcgaggctggggatcaacggcggctacgagttcgggtactcgaccgcgatgggcgggtctggcgctgtctctggcctcgggacgctcggcatcagccctttcttgaagtccggtaccgccggcggcgacgagaagcccagcggcggtcagtaggcggtccgtgtgatcgatccatcaaatcttgtcaggtttggtttggtttgttttgcatgctgcctacacagtgttctagtctgttttgcctttctgcttccgagtaattcgcgagtttggtagactacggttagtccgttttctttgctagatgcgcaatcatgcatgactaataagggtttctgcatgatgctagttggagcgtcggtcttgttcaaacaaagcattgcacaactgtttggatctatatattattgatgacttggtgcatgcatatgtacttgagcaccagagctttactgaacctctgatcaacagaatccagcatctcttttgtcaaactgttttgcttttatattgggtacttgtttagtatccacatgcacagcacaaggtgtggtgaacaaatctcttttcagatttccgatttgtgttgatcgtgtggtgaacctaaacagcttttttttttcttttttgatatgggtgttcctgtgaaatagtagttgaaacagaagagacaacatcggttagcaaatcctggagaatatcactatctctaggcagtcctctaattttcgagaagtcctttttaaaaaaaataaaaaataaaaactgtctatgagctagggcacagagaatttggtggacgtgcatcaatcatcatatatatatatcatatggtggcatgcctaatgtcatgtaggtgatgaatttcgcatatatctatctgcttaattgcatggcaatgtactagaaagtttcctgtgccaaaattaagcgattggtatcaagagcacgcacgcatgagtgtatcagtatccccagctagctagctagctatagctagcgacagtagtagctgctggtgctggatatatctctaggaaacaactgagatattttgtttccttttttaacgtaaaactggtagaaaactgccattttattaattttaattataaagagtagagctttgtataaattaaagcattgtcagctcgctactaacacaatattccattgtaatgtgctgtcagcttgaatgaactgtactaatataagtacccacaggccacatcatagagaagcttctgtgttgaaatagtagtaatattctttttgtctattcataagtggaaaaatgtacagtccgtgtcaaccctgcatttagcatgcagtttctatttacaacagtttgttggacagttcccatttttcgtgtctagcataacaatcatgctgtctacatttcagccatagtagtatatactacatttcatagccatatatatatattgttacgttttcagctatatatacataccaaccctgatagacatatttggaattgtttttttttcaggttactgatgtgaagaaacgaaaaaaaaaatccatgaagtgctgaagaccaagtggcgtgcaaggattatcctaaatgtccaccgttacgacggagggctaatttgcagtcgattctacatatgaagagtgcatgcatgcttatgtcgttgtttaattaattaattgcttgtttgttaattaccacctccctccctctagcctccatccacaagatcgtcagatatttcatgcaagcatccacaagtgccgtcttatgttgttgctgtactgcaaaacagaaaaaaattgcatgcttatccctgattttttttcttctcggacgactagcgcccctttttgtttctagtttctactattcgacctctagttctctactccatgtattgctaggttggctatactatgaagttgacctttgaggaggtgcagtttcttatggcctgatggatgcatctgtgattctgtgtgatatactgctgctgcgtgtatctttgctt

SEQ ID NO: 22 - DNA, 996 bp, Artificial Sequence
  cDNA of wild type allele of ig gene 1 of Sorghum bicolor
atggtcctacacaaggccccggtcagcaagggccttttcttcttcctttgcccccccagattgcgcgctctctccctcagagcctcacaactcggcgacgactacgccaagtgcccaggtcttctccaggcccggtcgctgctgctctacaccagtttcccgggtcttcttcggagttcggactcgtcttcgccggcgacgagcaccgccagcagacggatggcttcgtcggtgccggcgccatcggggtcggtgatcaccgtggcatcgtcttcttcctcagcagccgcggccgcggtgtgtggcacgggctcgccatgcgctgcgtgcaagttcctgcgccgcaagtgccagccggactgcgtgttcgcaccctacttcccaccggacaacccgcagaagttcgtgcacgtgcaccgtgtcttcggcgccagcaacgtgaccaagctgctgaacgagctccaccctttccagcgcgaggacgccgtgaactcgctcgcctacgaggccgacatgcgcctccgcgaccccgtctacggctgcgttggcgtcatctccatcctccagcacaacctacgccagctccagcaggacctcgcccgcgccaagtacgagctctccaagtaccaggcggcagcggcagcttctgcgtccacggcggcgcccaacgggccgcaggcgatggcagagttcatcggcaacgcgatgccgaacggcgcgcacaacttcatcaacattggacactcggcggcgctcggctccctcggaggctccgccaccgtgttcggccaggagcagttcgccaacgcgcagatgctgtccaggagctacgacggcgagcccatcgcgaggctggggatcaacggcggctacgagttcgggtactcgaccgcgatgggcgggtctggcgctgtctctggcctcgggacgctcggcatcagccctttcttgaagtccggtaccgccggcggcgacgagaagcccagcggcggtcag

SEQ ID NO: 23 - protein, 332 aa, Sorghum bicolor
MVLHKAPVSKGLFFFLCPPRLRALSLRASQLGDDYAKCPGLLQARSLLLYTSFPGLLRSSDSSSPATSTASRRMASSVPAPSGSVITVASSSSSAAAAAVCGTGSPCAACKFLRRKCQPDCVFAPYFPPDNPQKFVHVHRVFGASNVTKLLNELHPFQREDAVNSLAYEADMRLRDPVYGCVGVISILQHNLRQLQQDLARAKYELSKYQAAAAASASTAAPNGPQAMAEFIGNAMPNGAHNFINIGHSAALGSLGGSATVFGQEQFANAQMLSRSYDGEPIARLGINGGYEFGYSTAMGGSGAVSGLGTLGISPFLKSGTAGGDEKPSGGQ

SEQ ID NO: 24 - DNA, 3272 bp, Sorghum bicolor
cactttcatttcatatatccatccggccattattctctctccaaaacaaacacccgattaatcagctgatgtggcggagctgagacagacgaggaaaaaagcaaggggttgaagaaccggagcggccgccggcctcgtatcctgctgtgatcttcgcggaagggaaggaaaattaagggtacatgtattccttattcctctcactcctcgcccatagacggcatcatcggatccgaattgaatccttgatatgcttctcattctgccatttctcactgatcttagtttttttttctctctctcatctatctagctatttggttctcacgctgagatggatctcttaattagacctgctggatcttcctcctcctgtacagagatagatatagatggccgcctcgtcagcatcatcggtgccggtcgtctccacgggatccgtgatcactgtggcggcctcgccgccgccgccgccgcccactggtggtggtggtggtggtggcggctcgatctctgctggtagcacaggcgtcgtcgtcggcggcggcgcaggcacagggtcgccgtgcgcggcgtgcaagttcctgcggcgcaagtgccagccggactgcgtgttcgcgccctacttcccgccggacaacccgcagaagttcgtgcacgtgcaccgcgtcttcggcgccagcaacgtgaccaagatcctcaacgagctccacccctaccagcgcgaggacgccgtcaattcgctcgcctacgaggccgacatgcgcctccgcgaccccgtctacggctgcgtcggcgtcatctccatcctccagcaccgcctccgcctcgtccagcaggagctcgcccgcgccacctacgagctctccaagtaccaggtctgtgtctgtctgtgtgtgtgtggcttcctgtacctgtgcatgttctaacgtacgcgcgtccgctgcatctttcggtgtaggcggcggcggcggcggctgaagccgcatctgtgggcgtgggatcccatggtgatgcggcagcagggatggcggcagtggatttcataggcaacgcggtggccaacgactgcacgcaaaactttatcaatgtcgggcactccacggcggcggcggcgtcgatcagtggagctggcggcgggttcatgcagcagcaagacagcttcggcgcctccgtgcagatgctggccaggagctacgaaggtggagctgctgcgagggtgggcatgaacggaggaggtggatcaggctatggcggcttcgcgtactcgtcggccatgggcgtggtggggcatggtggcatagtgtctgctggtcttggcgggcagcagatcaacggtggacaacagttcttgaagcccagcacgaccgccggggatgaccagcccactgccacgcagtagtcaagtgtcgtctcatctttgtcaggttcggttctatgctgctatagaggatctttcttccgtaatttctgtgtattatttccctttatttacaatataccgtctactagcaataataatatatcatccatgaaaatagtatatattttgttgtagcaaggcgatgcatgcatgcttcacaggattgtgcaaatggcttaatttacctttttttctcgatcaggtgtggtcaactgtttttcattaattaagacggaagggattaatttgctaatttaattttaaaataaagttgcacgacactaatctgtttatcatctataaccattattcatcgctaatttcgtaatacccatgccttgctaaaactggagtctcaatagaaagctctatcatcttaactaacctgcctctgcgaatcctagatgttgcctataatctagaaaacatacttgtttagaaatgagagggtgagtggagactgcgataagataatcagagtttggacttttgtcccttcccccctaatccttttgttggttctaataataagtgtattacctatgactatcatatgaatgtatggagagataaagcttatttaatttcaacaaaaaaatataattcaaagtctaggtataaaatagatttaaagagcacaaaaaataaccaatttgcacacaaacaattataacagtaaaataaatatgatgtgtatagaaacatgatgttccataatttgagtctatgaattgtaaagatatatatcatgtgtatgcacatcaggacaatgttatattgtagaatgtgcctctaataaaaatattctcaggtgtgttcttcaaacttatatcaactcataaatactattaaatctcagaacttattgaaatgtagtgtttgaactatttgtgccttcaagaccattacattttagaagaaatttttgctttgatattacatgtcataacatgcattgaaatatatataacatttagatcctctataagaaagcacctaccaaaattttaatatcataacttgataagtaatttactataagagttgtagtatataaattatatataatatcaaacatataatatagtaattatttatttaatatatagctctgaatattattatctcagtccctctacattgtcataatatagggttgaccaaaatagaaatcatatttgcatggttcttattattaaacactatgatttattgttgtaaaagtaaatgacttatcatgtgaattaataagcaatgtcttacttggttttatcataacaattccaatgaattcattagtattcaaatgtgaagatctgtgtggcataccttctcttcaatattcatatatctaccatgcaaaaattacagactagagcaatgcatagatgtaaatgtcaatttgttttagattttagtactaataaaacattattgctatatgaaaaatgcaaatgatgtagtttacttatgagtttttgttgcgtctgtcttttatgcaagattttgtacttactaataatatatgcaggtcatgactactaaggcaaaagatgtcaacatgctaaattaaccgcttgatgaggacgtgaagactagattaagtgcaagatatatactagcatcctttataatatagatagtaattaaaaagaaacgagtcctttattttgtacttgttggtttaaattaattgttgatgcagttcacaaagtcgacttgaatattgctccatgtttgtgtttgagttatgtttgttgctttgtctctagatgtatggagctttgactttaagctagtgaaaataatataaagctaagttgttctatggagtttgacgttggtaata

SEQ ID NO: 25 - DNA, 924 bp, Artificial Sequence
  cDNA of wild type allele of ig gene 2 of Sorghum bicolor
atggccgcctcgtcagcatcatcggtgccggtcgtctccacgggatccgtgatcactgtggcggcctcgccgccgccgccgccgcccactggtggtggtggtggtggtggcggctcgatctctgctggtagcacaggcgtcgtcgtcggcggcggcgcaggcacagggtcgccgtgcgcggcgtgcaagttcctgcggcgcaagtgccagccggactgcgtgttcgcgccctacttcccgccggacaacccgcagaagttcgtgcacgtgcaccgcgtcttcggcgccagcaacgtgaccaagatcctcaacgagctccacccctaccagcgcgaggacgccgtcaattcgctcgcctacgaggccgacatgcgcctccgcgaccccgtctacggctgcgtcggcgtcatctccatcctccagcaccgcctccgcctcgtccagcaggagctcgcccgcgccacctacgagctctccaagtaccaggcggcggcggcggcggctgaagccgcatctgtgggcgtgggatcccatggtgatgcggcagcagggatggcggcagtggatttcataggcaacgcggtggccaacgactgcacgcaaaactttatcaatgtcgggcactccacggcggcggcggcgtcgatcagtggagctggcggcgggttcatgcagcagcaagacagcttcggcgcctccgtgcagatgctggccaggagctacgaaggtggagctgctgcgagggtgggcatgaacggaggaggtggatcaggctatggcggcttcgcgtactcgtcggccatgggcgtggtggggcatggtggcatagtgtctgctggtcttggcgggcagcagatcaacggtggacaacagttcttgaagcccagcacgaccgccggggatgaccagcccactgccacgcag

SEQ ID NO: 26 - protein, 308 aa, Sorghum bicolor
MAASSASSVPVVSTGSVITVAASPPPPPPTGGGGGGGGSISAGSTGVVVGGGAGTGSPCAACKFLRRKCQPDCVFAPYFPPDNPQKFVHVHRVFGASNVTKILNELHPYQREDAVNSLAYEADMRLRDPVYGCVGVISILQHRLRLVQQELARATYELSKYQAAAAAAEAASVGVGSHGDAAAGMAAVDFIGNAVANDCTQNFINVGHSTAAAASISGAGGGFMQQQDSFGASVQMLARSYEGGAAARVGMNGGGGSGYGGFAYSSAMGVVGHGGIVSAGLGGQQINGGQQFLKPSTTAGDDQPTATQ

SEQ ID NO: 27 - DNA, 1758 bp, Brassica napus
ggaaatcaattccctctagtactactaaatcctattactattaaggtatgtttatagttatttattactagtatataaatgtttttaaaataaacatcattaattagtaatagtcgtatcatatttatgagcgtatttttattacagaataatattatatttaattgagtaaaaatatagtaaaatttaagcacaagatgagctttgcccatcgagagcctcaaaacccccaaaagtctcttctagcttcacgagatccaatctcttgctttttagttattaattacatcttcttccttctcttctctatttgccctctctctcacacctacctatctaagcaacacaaagaacaccattgatctcatctattttattccttatatttcctcggttcatccttaagcatttcatctataaggtacataattaaaatttctttccctctttcgttgttcatttatagacatcataactttctcaggttatctctagacttgaaacttttagaggtctagtttccaaaatttgactagcaatcatatagctacaagtcactctatatatacgcatagcttaggtctttacgacatgattttttatttcctccaagtatgtagtatatttgatgcaaacctttttctttgactttataaaagcaccttaatgtttctcatctgttcccctatttgttttaaacagtttgacaagaaggttcaagtgcatataaaaaatatatacatgcttacatgtgtatttgtacataaaaaaattgctactaatatctttaatgctattaattacaaagtttcctctcattttacagtttgttttccaaatagcagctaagcgaaagcagagaagatctccatttactttccctccagagaaacagaatccttattaaaagaggagtctaatttattcgcaggtaagccttcttattctagagatttcaacatctccaaatatatttctctgtcacgtatttcatattagtttcattacaaaaacaaaaaaaaaactcaaaaatcttcaatcttatcttttcacaaatgatacataagttcttcctccaataagaaaaaaaaaaacagaaaccctagctagctttttcaattttaatggcggctttgtgtaggaagaaaaatggcatcttcatcaacaaactcaccatgcgcagcctgcaaattcctccgtagaaaatgccaaccggagtgtgtattcgctccatatttcccacctgaccaaccacaaaaattcgcaaacgttcacaaagtctttggagcaagcaacgtcacgaagctcctcaacgagcttcacccttcgcaacgtgaagacgcagtgaactctttagcttatgaagccgacatgcgtcttcgtgatccagtctacggctgcgtcggagtcatctctctcctccaacatcagcttcgccagcttcagatcgatctcagctgcgccaaatctgagctttccaagtaccaaagcctcggtatactcgccgccacccatcagagccttggaatcaacttactcgcaggcgccaacgcagatggatcgacgacggccacagtgagagaccatcattatcatcaccaccagtttttccctagagagcagatgtttggtgggttagatgttcctaccggtaacaactacgacggagggattcttgcgattggacagatcagtcagtttcagcagccgagagcagccgctggagatgatggtcgccgtactgttgatccgtcttga

SEQ ID NO: 28 - DNA, 609 bp, Artificial Sequence
  cDNA of wild type allele of ig gene 1 of Brassica napus
atggcatcttcatcaacaaactcaccatgcgcagcctgcaaattcctccgtagaaaatgccaaccggagtgtgtattcgctccatatttcccacctgaccaaccacaaaaattcgcaaacgttcacaaagtctttggagcaagcaacgtcacgaagctcctcaacgagcttcacccttcgcaacgtgaagacgcagtgaactctttagcttatgaagccgacatgcgtcttcgtgatccagtctacggctgcgtcggagtcatctctctcctccaacatcagcttcgccagcttcagatcgatctcagctgcgccaaatctgagctttccaagtaccaaagcctcggtatactcgccgccacccatcagagccttggaatcaacttactcgcaggcgccaacgcagatggatcgacgacggccacagtgagagaccatcattatcatcaccaccagtttttccctagagagcagatgtttggtgggttagatgttcctaccggtaacaactacgacggagggattcttgcgattggacagatcagtcagtttcagcagccgagagcagccgctggagatgatggtcgccgtactgttgatccgtcttga

SEQ ID NO: 29 - protein, 202 aa, Brassica napus
MASSSTNSPCAACKFLRRKCQPECVFAPYFPPDQPQKFANVHKVFGASNVTKLLNELHPSQREDAVNSLAYEADMRLRDPVYGCVGVISLLQHQLRQLQIDLSCAKSELSKYQSLGILAATHQSLGINLLAGANADGSTTATVRDHHYHHHQFFPREQMFGGLDVPTGNNYDGGILAIGQISQFQQPRAAAGDDGRRTVDPS

SEQ ID NO: 30 - DNA, 1754 bp, Brassica napus
ggaaatcaattccctctagtactaccaaatcctattactattaaggtatgtttatagttatttattattactagtatataaatgtttttaaaataaacatcattaattagtaatagtagtaatgatttatgagcgtatttttattacagaataatattatatttaattgagtaaaaatatagtaaaatttaagcacaagatgagctttgcccatcgagagcctcaaaacccaaaagtctcttctagcttcacgagatccaatctcttgctttttagttattaattacatcttcttccttctcttctctcttcccctttctctcacacctacctatctaagcaacacaaagaacaccattgatctcatctattttattccttatatttcctccgttcatccttaagcatttcatctataaggtacataattaaaatttctttctctctttggttgttcatttatagacatcataactttctcaggttatctctagacttgaaacttttagaggtctagtttccaaaatttgactagcaatcatatagctataagtcactctatatatacgcatagcttaggtctttaggacatgattttttatttcttccaagtatgtagtatatttgatgcaaacctttttctttgactttataaagcaccttaatgtttctcatctgttcccctatttgttttaaacagtttgacaagaaggttcaagtgcatataaaatatatatatacatatatgtgtatttgtacataaaaaattgctattaatatctttaatgctattaattacaaagtttccactcattttacagttttttccaaatagcagctaagcgaaagcagagaagatctccatttactttccctccagagaaacaaaatccttattaaaagaggagttcaatttattcgcaggtaagctttcttattctagagatttcaacatctccaaatatatttctctgtcacatatttcatattagtttcattacaaaaacaaaaaaaaaattcataaatcttcaaatcttatcttttcacaactgatacataagttcttcctccaataagaaaaaaaaaaaatcagaaaccctagctagctttttcaattttaatggcggctttgtgtaggaagaaaaatggcatcttcatcaacaaactcaccatgcgcagcctgcaaattcctccgtcgaaaatgccaaccggagtgtgtattcgctccatatttcccacctgaccaaccacaaaaattcgcaaacgttcacaaagtctttggagcaagcaacgtcacgaagctcctcaacgagcttcacccttcgcaacgtgaagacgcagtgaactctttagcttatgaagccgacatgcgtctccgtgatccagtctacggctgcgtcggagtcatctctctgctccaacatcagcttcgccagcttcagatcgatctcagctgcgccaaatctgagctttccaagtaccaaagcctcggtatactcgccgccacgcatcagagccttggaatcaacttactcgcaggcgccaacgcagatggatcgacgacggccacagtgagagaccatcattatcatcaccaccagtttttccctagagaacagatgtttggtgggttagatgttcctaccggtaacaactacgacggagggattcttgcgattggacagatcagtcagtttcagcagccgagagcagccgctggagatgatggccgccgtactgttgatccgtcttga

SEQ ID NO: 31 - DNA, 609 bp, Artificial Sequence
  cDNA of wild type allele of ig gene 2 of Brassica napus
atggcatcttcatcaacaaactcaccatgcgcagcctgcaaattcctccgtcgaaaatgccaaccggagtgtgtattcgctccatatttcccacctgaccaaccacaaaaattcgcaaacgttcacaaagtctttggagcaagcaacgtcacgaagctcctcaacgagcttcacccttcgcaacgtgaagacgcagtgaactctttagcttatgaagccgacatgcgtctccgtgatccagtctacggctgcgtcggagtcatctctctgctccaacatcagcttcgccagcttcagatcgatctcagctgcgccaaatctgagctttccaagtaccaaagcctcggtatactcgccgccacgcatcagagccttggaatcaacttactcgcaggcgccaacgcagatggatcgacgacggccacagtgagagaccatcattatcatcaccaccagtttttccctagagaacagatgtttggtgggttagatgttcctaccggtaacaactacgacggagggattcttgcgattggacagatcagtcagtttcagcagccgagagcagccgctggagatgatggccgccgtactgttgatccgtcttga

SEQ ID NO: 32 - protein, 202 aa, Brassica napus; identical to SEQ ID NO: 29

SEQ ID NO: 33 - DNA, 465 bp, Artificial Sequence
  cDNA of wildtype CENH3 protein derived from Beta vulgaris
atgagagttaaacacactgctgccaggaaatcaaccaccaatggtcctcgttcaaaggctcagaaatctccgcgcagtttgcaatcaccacaatcgccttctagtagttcaaagcgcaaatcacgcagaaacactgatgcaactcctcaaaagaagaaggcttaccgccgtaagccgggcactgtggcactctgggaaatacgcaaatttcagaagtcattcaagcccttgattcctgctgcgcctttcattcgaacagtgagagagattactcaccagtttgctccttatgttggtcgttggcaagctgaagctctgatggcccttcaagaggctgcagagaattttattgtccgtttgtttgaagatggtatgctttgtgcaattcatgccaaacgagttacactcatgaaaaaggatttggagctcgcgcgaaggattgggggcagagagaggggatggtaa

SEQ ID NO: 34 - protein, 154 aa, Beta vulgaris
MRVKHTAARKSTTNGPRSKAQKSPRSLQSPQSPSSSSKRKSRRNTDATPQKKKAYRRKPGTVALWEIRKFQKSFKPLIPAAPFIRTVREITHQFAPYVGRWQAEALMALQEAAENFIVRLFEDGMLCAIHAKRVTLMKKDLELARRIGGRERGW

SEQ ID NO: 35 - DNA, 2064 bp, Artificial Sequence
  cDNA of Zea mays CENP-C protein
atggacgccgccgaccctctctgcgccatcacctcgcctgcgcggctccttccgcggacccttggccccaccccctcgaaggtccgcggtgcgctcctcgaggccatcgcactcgcccggtcgcggaaagggtctgaggagctggttaagcaggctgcaatggtgctgaaggagcacggggacatccagactctctaccatgatgatgcaatgaaagccatgccccccacaaatgtcagcaaagggcaacagggaagaagggcagcactggatcgcaaacgggctcggttcactatgaaggacactggaagcaaagcggtgcctgttgtggatcgatctaagttattgaatatttcggatccagtcgcattcttcatggccttggatcggcttgaagaagctgaggatgagatcaaacggctaaatggagaggcagaaaagcgcatattgatttttgatcctttagacgaacccattagacagcctggtttgcgtgggagaaaatcattccgcagtttcaaagtaactgaggatgctactactcaagatcccattgaggtactggcttcccaaacagcaactatgacaggatctcagttctcacaggatgttatgcatgctgttgctgacaaaaatgaacagtctgttccttcacgatctggtgaagctatttcagatgtttcaggaaaagaagattcattagctgagaaggatggccgtgatgatttggcttatttactgacctcgatccaagatttggatgaatctgaagaggaagcatttatccggaagaccttagggcttaaagaaataaggaagggaagagttagtctctgtaactccattcctggagtcaggtccttaagaagcgatactgaacaaaaaggttcaatgaaggttcgccctccagaaagtcatttgcctcagactcgccaagatcgaatttcagagttggagaaacatttatttcctggaggtgcagcaaatgctaaatgcacagatgatgaatctgaagggtcaccagatattgtgatgggtgaaccatcattagtgcatgattcttccgaggttctgatgaccaatgagaactctactggaagtgaaattgacaggaggaccccaaatctgtgtgccagagcagcagaccacgctcttgatcctgaaccaaacttgcctgatcatgcatacgaaaggcaaaccagaggctcctcacttggtttgtgcaaaaacacagaagttgccaatgaacacgaggaagatgatgtacacatagaatatccaactactggcgggtctacttatcaaacagaagtttcttctcatgatttggagggcagcttgacagaagaattggtcagtgaaccaggtaggcatgcggctccagatgatatctataggacttcacatgcagctgaggatagcattcaacatctagaagtggtcaaacagggtggtgttctacaagacaagtcaagccagtcgttggagatgcctctggaagatattgatccactatatcggcctcagatgcatggtggaagcactaagaaatcgacacctgttctgtgcaatgcactgtccccgatcgaacagaagcaacaagcagctcaagaagggaaaaggaagcaacaggcaaagagggtaaaaaaagtgtctgatgaatctagccatgcactggaaatatcccaagcaaatttcgaaccggagaatgaacctcataatgttgacgtgaacatggagcaaccgacagttatgagtagcgccaaggggcaaaagggagctcaaaggagaaacaagaccaaaaaattgaatcaaagaaaaatccttggagatgttgaccttgcgcggcagtctggagtgagaagaagctcaagaatacgctcgaggcctttggagtattggcttggtgaaagattactatatgggccgatacaggacactttgcctgcagttattggcatcaaagcatactctcctggccaggatggcaagaaaacattgaaagtgaaatctttcgtgtctgaacagtattcagatcttgtcgctcaatttgcgaagtactaa

SEQ ID NO: 36 - protein, 687 aa, Zea mays
MDAADPLCAITSPARLLPRTLGPTPSKVRGALLEAIALARSRKGSEELVKQAAMVLKEHGDIQTLYHDDAMKAMPPTNVSKGQQGRRAALDRKRARFTMKDTGSKAVPVVDRSKLLNISDPVAFFMALDRLEEAEDEIKRLNGEAEKRILIFDPLDEPIRQPGLRGRKSFRSFKVTEDATTQDPIEVLASQTATMTGSQFSQDVMHAVADKNEQSVPSRSGEAISDVSGKEDSLAEKDGRDDLAYLLTSIQDLDESEEEAFIRKTLGLKEIRKGRVSLCNSIPGVRSLRSDTEQKGSMKVRPPESHLPQTRQDRISELEKHLFPGGAANAKCTDDESEGSPDIVMGEPSLVHDSSEVLMTNENSTGSEIDRRTPNLCARAADHALDPEPNLPDHAYERQTRGSSLGLCKNTEVANEHEEDDVHIEYPTTGGSTYQTEVSSHDLEGSLTEELVSEPGRHAAPDDIYRTSHAAEDSIQHLEVVKQGGVLQDKSSQSLEMPLEDIDPLYRPQMHGGSTKKSTPVLCNALSPIEQKQQAAQEGKRKQQAKRVKKVSDESSHALEISQANFEPENEPHNVDVNMEQPTVMSSAKGQKGAQRRNKTKKLNQRKILGDVDLARQSGVRRSSRIRSRPLEYWLGERLLYGPIQDTLPAVIGIKAYSPGQDGKKTLKVKSFVSEQYSDLVAQFAKY

SEQ ID NO: 37 - DNA, 2085 bp, Artificial Sequence
  cDNA of Sorghum bicolor CENP-C protein
atgaatgccgccgaccccctctgcgccatctccttgcctgcgcgactccttccgcggacccttggccccgcctccgccaccgggccttccccctcgaaggccggcgacgcgctcctcgagaccatcgcagtctcccggtcgctgaaagggtctgaggagcttgttaagcaggctaaaatggtgctgaaggagcacggggacatccagcctctctaccatgatgatggagtgcaagtgaggccccctgtaaatggcagcaaagagcaacagggaggaaggccagcactgaaccgcaaacgggctagattcactatgaaggacactgcaagcaaaccgacgcctgttgtggatcaatctaagttgacgaatatttcagatccagtcgaatactttatgaccttggaccagcttgaagaagctcaggaggagatccaacggctaaatggcgcagcagaaaaatgcgtattaaaatttgatcctgtagatcaaccaaagagacagcctggtttatgtgggagaaaatcaatccgcagtttcaagataattggggatgctgatactcaagatcccattgaggtaccggcttcccaaacaacaactttgacaggatctcaggtctcacaggatgttgtgcatgctgttgctgacaaaaatgaacaatgtgttcgttcaagttctgctgaagctatttcagggaaagaagattcattaactcagaaggatggccgtgacgattttacttatttactgaactcactccaacatttggatgaatctgaagaggaagggtttatcctcaagaccttagggattggagagccaaggaaggaaagagttagtttccgtaactccattcctggagttaggtccataagaagcaataatgaacaaaaaggttcaatgagggctcaccctttagaaagtcctttgcttcagcctcttcaggatcgaatttcagagttggagaaacatttatttcctggaggcgcagcagatgctaaatgcacagatgatgaatctgaaggttcaccagatattgcgatgggcgaaccatcattagtgcatgattcttctgatgttccgatggttgatgagaactttactggaagtgaaattgacagggagaccccaaatctgggtgccagagcagcagaccatattcttgatcctgaaccatccgatcatgcatatgaaaggcaacccggaggttcctcagttggtttgtgcagagacacagaagttgccaaagaaaacgaggcatgcaggaggagcaatatttctatggaggaagatgatgtgccaatagactatccaactattggcagatctaccagtgaaacagaagcttcttctcatcatctggagggaaggtcgacagaagaattgggtatcaataggactttacatacagctgaggatagcattcaacatctagaagtggtgaaagaaggtggtgttctacaagataagtcaagccagtcattggagatgcctctggaagatattgatccagtgaatcagcctcagatgcatggtggaagcactaagaaattggcacctgatttgtgcaatgcactgtccttaaccaaacagaagaagcaacaagcagcccaagaagggaaaatgaagaaacagtcaaagaggggcaaaaaagtggctgatgaatctagccttgcactggaaatatcacaagcaaatttggattcggagaatcaacctcataatgatgatcagaacattgagcaacatacagtgttgagtagcacactgtcgccaaaccacgataaggggcaaaagggagctcaaaggacaaacaagaccaaaaaactgaatcaaagaaaaatccttggagatgctggccttgcacagccgtctggagtgagaagaagcacaagaacacgctcaaggcctttggagcattggcttggtgaaagattactatatgggcctataaatgacactttgcctgtagttattggcatcaaatcatactctcctggccaagacggcaagaaaacattgaaagtgaaatctttcgtgcctgaccagtattcagatcttgtcgctaaatccgcgaagtactaa

SEQ ID NO: 38 - protein, 694 aa, Sorghum bicolor
MNAADPLCAISLPARLLPRTLGPASATGPSPSKAGDALLETIAVSRSLKGSEELVKQAKMVLKEHGDIQPLYHDDGVQVRPPVNGSKEQQGGRPALNRKRARFTMKDTASKPTPVVDQSKLTNISDPVEYFMTLDQLEEAQEEIQRLNGAAEKCVLKFDPVDQPKRQPGLCGRKSIRSFKIIGDADTQDPIEVPASQTTTLTGSQVSQDVVHAVADKNEQCVRSSSAEAISGKEDSLTQKDGRDDFTYLLNSLQHLDESEEEGFILKTLGIGEPRKERVSFRNSIPGVRSIRSNNEQKGSMRAHPLESPLLQPLQDRISELEKHLFPGGAADAKCTDDESEGSPDIAMGEPSLVHDSSDVPMVDENFTGSEIDRETPNLGARAADHILDPEPSDHAYERQPGGSSVGLCRDTEVAKENEACRRSNISMEEDDVPIDYPTIGRSTSETEASSHHLEGRSTEELGINRTLHTAEDSIQHLEVVKEGGVLQDKSSQSLEMPLEDIDPVNQPQMHGGSTKKLAPDLCNALSLTKQKKQQAAQEGKMKKQSKRGKKVADESSLALEISQANLDSENQPHNDDQNIEQHTVLSSTLSPNHDKGQKGAQRTNKTKKLNQRKILGDAGLAQPSGVRRSTRTRSRPLEHWLGERLLYGPINDTLPVVIGIKSYSPGQDGKKTLKVKSFVPDQYSDLVAKSAKY

SEQ ID NO: 39 - DNA, 1797 bp, Artificial Sequence
  cDNA of Arabidopsis thaliana KNL2
atgacggaaccaaatctcgacgaagatggttccaagtcgtctttccagaaaacggtggtgctgagagattggtggttgataaaatgcccaaaggaattcgaagggaaacaatttggtgttgctggattcgaagagtccgtcgagacaagagcaatgcgtgtgtttacatcttcaccaatcaccaaagctttggatgttttcacactcttagcatctgatggaatctatatcactctcagaggttttcttaacaaagaacgcgttcttaaaaatggatttaaccctgagatttctcgtgaattcatcttcgggtttcctccttgttgggaacgagtttgtaacagttgctttgaaggagactcttttggcactgatgttaataccgtcccttcgactatcgagaaagcttgtcctcccattttatcaccttgtaagtactctaacaggaatcttaaggataatccagctgagagcagagagaaaagcaatgtgactgagactgatattgcagagattaatgataaaggtggttctggagcaagagatataaaaactgcaaggagaaggtctcttcatctgcaaataaaaaggatacttgaatcgagtaaagtccggaagactgctaatgatggagatcatggtagtgaatttttgaatacggctaagcgcggtgatgtagaaagagatggatgtgaggttatcaataatgaagacagtgaatggaaactcgatgaaagtgaagtccagaatctttgtaatgatggagataatggtagtgaaggtttcattaaggctaagagcagtgatgtagaaaaagataagagcgaagctatcgataatgatgtgatatctccagcggttggaagtggtattaagcataccggtgcagataatgttgataaagtgacaagtgcaagtgctactggagaatcacttacttcggaacagcaaaatggtttacttgtaacaacagcatctccacattccctgcttaaagacttagccaagagtagcaaacctgaaaagaaaggaatatccaagaaaagtggcaagatcctcagaagtgacgacaatgtagtagatcccatgaattactctgggacgaaagtcaaaagtgcggaaaacaaaaggaaaatcgatgcgagtaaactccagagtccgactagtaatgttgcagaacatagtaaggaaggtttaaataatgctaagagcaatgacgtagaaaaagatgtatgtgtggctatcaataatgaagtgatatcaccagtgaagggatttggtaaaaggctttctggtacagatgttgaaagattaacaagtaagaatgctactaaagaatcactgacgtcagtacagcgaaaaggtagagtgaaggtatcgaaggcatttcaagatcccctgtcgaaaggaaaatcaaagaaaagtgagaagacccttcaaagtaacagcaatgttgtagagcctatgaatcatttcaggtctgaagctgaagaagctgaagaaaacttgtcatgggaaaaaataaagaggaaaatcgactttgatgtggaggtaacaccggaaaaaaaagtgaagcagcagaagaccaatgcggcgtctactgattcattgggacagaaacggtcaagatcaggaagggtgcttgtgtcatcactagagttttggcgtaaccaaattcctgtttatgatatggatcggaaccttatccaagtaaaggatggtagtgagactaactccgctccatctaaaggaaaaggatcggattctcgaaagcgaagaaacttgaaaatcaaataa

SEQ ID NO: 40 - protein, 598 aa, Arabidopsis thaliana
MTEPNLDEDGSKSSFQKTVVLRDWWLIKCPKEFEGKQFGVAGFEESVETRAMRVFTSSPITKALDVFTLLASDGIYITLRGFLNKERVLKNGFNPEISREFIFGFPPCWERVCNSCFEGDSFGTDVNTVPSTIEKACPPILSPCKYSNRNLKDNPAESREKSNVTETDIAEINDKGGSGARDIKTARRRSLHLQIKRILESSKVRKTANDGDHGSEFLNTAKRGDVERDGCEVINNEDSEWKLDESEVQNLCNDGDNGSEGFIKAKSSDVEKDKSEAIDNDVISPAVGSGIKHTGADNVDKVTSASATGESLTSEQQNGLLVTTASPHSLLKDLAKSSKPEKKGISKKSGKILRSDDNVVDPMNYSGTKVKSAENKRKIDASKLQSPTSNVAEHSKEGLNNAKSNDVEKDVCVAINNEVISPVKGFGKRLSGTDVERLTSKNATKESLTSVQRKGRVKVSKAFQDPLSKGKSKKSEKTLQSNSNVVEPMNHFRSEAEEAEENLSWEKIKRKIDFDVEVTPEKKVKQQKTNAASTDSLGQKRSRSGRVLVSSLEFWRNQIPVYDMDRNLIQVKDGSETNSAPSKGKGSDSRKRRNLKIK

SEQ ID NO: 41 - DNA, 2264 bp, Brassica napus
cgtggaattcttaccactaaactacagccacttcattggttcaaccctcaaaactatattgcttaattaaaaatgcttcccgcgcagtaatgaagaaatctcgcgccgaagtcttctttttcattcccactgttctaaagccctaactcttcctcaatcaatcgagaaagtattatctctctgctcctcctcctccatccatggctgacaatcctaatcccaatccagacgaggaagatgtgtcgtattacgagaaaacggtgagtgaggctattttcttaaaaacacttgctcggctctcctgattcctttaaactttgaatgtaaaaaaaaaggtggtcctgagagactggtggctgatcaaatgtccaattgaattccaaggcaaacgatttggcgttgctggtacccagattgctgagtaagttaggagtaaggagtaaggaggaggagtcaaaccctttttctatttctgctttacttttaaagtattcttattgatgtttgggaattgggaacaggacaggagcagtgagggtgtttacatcatccccaatcctcaaagcctttgatgttttcacactcgaagcttctgacggagtctgcatcgtcctacgtggctttctcaacaaaccacgccttgttcactctggattcctccctcaggtaattttatatactaattgaaagctattgttaatatgcctattaatatgccttttgtttgtctgtagatttgcagtgagttcatcttggggtttcctccttactgggaatcaaaatgtaacctttccttcgtaggactgccttctggatcagcttctatcaataaaggtttgatcttttttgtagcaacatatctgttttatgttttctgttcttataatgcttatgattaagctcttactgcagcttctggtaccattttatcaccttgtaacaacgacaagaaacggaatctagaggatactccagctcggagaagagtagttaaaaccattgtcactgctaagaagaagaagcagaacactgtggagattagtgatagaccttcaaggaaaaagtctcttcgtctgcagtccaaatctgttgaattgatgagtaaactccagactacttcttctactaatgatggtttggacaagagtgctaagtgcagtgatgatgtagagaaaacagatgaatctgaggttaccaataaccaagttgatggatgtggtaagaagcgtgtgaatcatcagtctgggaccaaagtcaaaaggaaacttgatgttagcgaactccagaagaatcctactactaatgatgaatctatggataatgaagagatatcaccttcaccagtggatgggtgtggtactaatagcaaaaagataacaagtaagaatgctacactgacttcagaagagcgaaatggtaagctcaaggtaactaaaacatctctaaagaatgggaagaaaagtgagaagatccttgaaggtgatttggatgatgtagtagtagagcctatgacgacgactcattcaaggtcctccaaggttaaacacaacttatcagttgggaaaactatcaggaagatcgactttgatctggaggtaactaacttttttttgtcacaatctcatattcttgttatcaaaatattcatcataatgatacccccgttgttatgtaatgcgaaggtaacaccagagaaagatgcgacgaaacataacaagaccaattcaatgtctgctgattcattaggacagaaacggtctagatcaggttaattgataaagagtaactttcagagtctcagttgagagagcattcactaaagaaagaaagtttccatttcaattaattgcaggaagggtgctagtgtccccactagagtactggcgcaaccaacttcctgtttatgataaggtttgtgattatgtttactgtattaacactaaccatgagaacatgatcttaactgtttctgttctggttttgtaggatcggaatcttatccaagtaaacgaaggtcgtcagactaactccacttcgtctaaaggtttgttcttcttttcttaaaagaataaaagagggcttccactataattatacaaactgctcttcttgatagggaaaggagatccgtttctcgaaagccaagaagatgaataatcaagtaaacagctttcaactactttggtaattagtgttatggtctctgaacatgtaatcatcttgagcttattgttggatcgtttaactcttaggatttggagcta

SEQ ID NO: 42 - protein, 426 aa, Brassica napus
MADNPNPNPDEEDVSYYEKTVVLRDWWLIKCPIEFQGKRFGVAGTQIAETGAVRVFTSSPILKAFDVFTLEASDGVCIVLRGFLNKPRLVHSGFLPQICSEFILGFPPYWESKCNLSFVGLPSGSASINKASGTILSPCNNDKKRNLEDTPARRRVVKTIVTAKKKKQNTVEISDRPSRKKSLRLQSKSVELMSKLQTTSSTNDGLDKSAKCSDDVEKTDESEVTNNQVDGCGKKRVNHQSGTKVKRKLDVSELQKNPTTNDESMDNEEISPSPVDGCGTNSKKITSKNATLTSEERNGKLKVTKTSLKNGKKSEKILEGDLDDVVVEPMTTTHSRSSKVKHNLSVGKTIRKIDFDLEVTPEKDATKHNKTNSMSADSLGQKRVLVSPLEYWRNQLPVYDKDRNLIQVNEGRQTNSTSSKGLFFFS

SEQ ID NO: 43 - DNA, 2350 bp, Brassica napus
ggaattcttaccactaaactacagccacttcgttggttgaaccctcaaaactatattcctttattacaaaatgcttcccgcggagtaatgaagaagatctcgcgccgaagtctttttttcattttctaaaaagccccaactctttcctcaatacagttcagttcgactatctctgtctctctctctctcactctcactctctcaatcaatcgagaaagtactccctctgctcctcctccatccatggctgacaatcccaatccagacgacgacgatgtctcgtattaccagaaaacggtgagtgaggctactttcttaaactctaatgagtcttacatcatcttcatctgtgtggggttttaatgtaaaaaaaaaggtggtcctgagagactggtggctgatcaaatgtccaattgaattcgatggcaaacgatttggcgttgctggtacccagattgctgagtaagtaaggagtagtgaaaccttttctcaattttcgtttttttccattctttttaaaagtttgggaattgggaacaggacaggagcagtgagggtgtttgcatcatccccaatcgtcaaagcctttgatgttttcacactcgaagcttccgatggagtctgcatcgtcctacgtggctttctcaacaaacaacgccttgttctatctggattcctccctcaggtctatatatatatatatatatatactagttgaaagctattattaatatgccttttgttttcctgtagatttgcagtgagttcatcttggggtttcctccttgttgggaatcaaaatgtaacctttccttcgtaggactgccttctggatctgcttctatcaataaaggtttgatcttttattagcaacatctctgttattgtgtgtttttccttttttttcctgatgcttatgattagctttcattgcagcttctggtgccattttatcaccttgtaacaacgacaagaaacggaatctagaggatactaaaagcactgtcactgctaagaagaagaagaagaacacagtggagattagtgataaaccttcaaggaaaaagtctattcgtctgcagtccaaatctgttgagttgatgagtaaagtccagactacttcttctactaatgatgttagtgatggtttggacaagaggggtaagagcagtgatgatgtagagaaaacagatgaatgtgaggttatcaataaccaagttgatggcaatgtagtagagcttgtgaatcatcagtctgggaccaaagtcaaaaggaaacttgatgttagccaagtccagaagaatcctactactaatgatggcgtcgaaagagatgaatctatggttaatgaagagatatcaccttcaccagtggatggatgtggtactaatagcaaaaagataacgagtaagaatgctacactgacttcagaagagcgaaatggtaagctcaaggtaactaaaacatctctgaagaatggaaagaaaagtgagaagatccttgaaggtgatttggatgatgtagtagtagagcctatgatgactactcattcaaggtcctccaaggttatacacaacttatcagttgggaaaactatcaggaagatcgactttgatgcggaggtaactaactttgttcccctccccatttttttttgtcacaatcacatattcttgttatcaaaatattcaaatgttaatcataatgatacctccgttgttatgtaatgcgaaggtaacaccagagaaagatgcgacgaaacagaagaccaattcaatgtctgctgattcattaggacagaaacggtcaagatcaggttaatttgatagagagagtagaagaacttattcactttcagaggctcagttgagagagcgttcactaaagaaaacaagtttccattttaattgcaggaagggtgctagtgtcaccactagagtactggcgcaaccaacttcctgtttatgataaggtttgtgatgatgtttactgaataacactaaccatgatgaggacatgattttaactgtttctgttctggttttgtaggatcggaatcttatccaagtaaacgaaggtcatcagactaactccacttcatctaaaggtttgttcttcttttcttaaaagaataaaagagggctttcactataattatacaaactgctcttcttgatagggaaaggatccgtttctcgaaagccaagaagatgaataatcaagtaaacagctttcaactactttggtaattagtgttatggtctctgaacatgtaatcatcttaggcttattgttggatcgtgatcgtttaacttcttgtttttgcctaaatagagaccttagagttctt

SEQ ID NO: 44 - protein, 434 aa, Brassica napus
MADNPNPDDDDVSYYQKTVVLRDWWLIKCPIEFDGKRFGVAGTQIAETGAVRVFASSPIVKAFDVFTLEASDGVCIVLRGFLNKQRLVLSGFLPQICSEFILGFPPCWESKCNLSFVGLPSGSASINKASGAILSPCNNDKKRNLEDTKSTVTAKKKKKNTVEISDKPSRKKSIRLQSKSVELMSKVQTTSSTNDVSDGLDKRGKSSDDVEKTDECEVINNQVDGNVVELVNHQSGTKVKRKLDVSQVQKNPTTNDGVERDESMVNEEISPSPVDGCGTNSKKITSKNATLTSEERNGKLKVTKTSLKNGKKSEKILEGDLDDVVVEPMMTTHSRSSKVIHNLSVGKTIRKIDFDAEVTPEKDATKQKTNSMSADSLGQKRSRSGRVLVSPLEYWRNQLPVYDKDRNLIQVNEGHQTNSTSSKGKGSVSRKPRR

SEQ ID NO: 45 - DNA, 4846 bp, Sorghum bicolor
ccactataataatttattctcttaaaattatctgttaaatatttaacagtattttttttcttttgctaccatgagcttttcagccacgcgctgaacagtgccgctacggcgctactacgtggagaggaaacgaaaaaagaaagagagaaaaaaaaaagaccccgctccattccgctactggcctactgccgtgtttggaaatgaagcccctacccgtacccgaggcggggagccctcaccgccgcggcatgccaccgtcgctgctcagcccttcctcccgcagcgcggttcccgccgccgccgacggcgaccatgacgccgccgtctccgagcacgcctgcgtacgtacgcccgtcgcccgccctaatgctgctagggcgcgtgtcgattcttttcctttttctttcccccgcccctcttccgtttcccctttactggctgagacgcgtgtgccctgacgcgatttggtgctgcgcgcgcgcgcgcgtgcgtgcgtggtggcctgcatttcgggtgtctaggtcacgctgtccgagtggtggctggcaaccgcggaaggggacgaccagaagatcgctgtcgccggcacattcgaacggtaagcatttcttcggaggggaaggggaaactagacgagccatctctgaaggtccgggcttgagtgggtggcaactagtttcgtttgtttttctgggggttcgatcttgttgttgaggagtggcatttggagtcctccggggggtatgcttcaaatttgttattgcaaattgatacctagattgactgattttgagttgacgccatgcactcctaaagaataggccaatttaattttctcagactattcttttgtcaaattgattcaacaaaccaatgtttatgccttttgtcctatgattcattcttgacatatgagggctattaatgccttgactgaagcatggtgaaacttctataagtcatgctgcttgtgtgtttaagcatgattaccgttttctgaagtaggctgcttttcctcttgaaagagctacggagtctttcgctgataacaataattatctattgcctgctctgacagcaatcaaacagttcaggaatactctcctgcacctattgccaagcgtcatacgtcttctgttcttgagactgaggaaggaactgtacttcgcctccatggtttacacaatgttttgcgaacctatcacaatggatattcagctaaggtatgcacatcctgttttctgtttcttgtttccggcaccaattgaatgcctatttggacaactttttaagtgaatacagtatcaagagcagatgatatcttttgttttctctatccatccagtgattctatttctgtgttcctctggctgaaagttattctcgattcgagcaatatctaccatctagaaaatatcatgtaccaaaaaaatgttttgccaaaatgcatgtccaaaaggattattcagccatgcacagctctcacaaatgccttggtcttgactgcaaaccatgcatgcaggtctacagtgagttcctgaatgggtttcccgactggtggcaaagttgcaagccgtgtaatcccaagctgatgaactcgcacacagaatgttgttcttctaatgccagcaattctggagtggactccactcaattttacctggagagatatatgcaggggagacgtttggattcatatggaacatatttgattagcaaatttcctgacattttggcaagtttcttacacaatgatgctgtgttccaaaaatcatcacatttattaaatggaaagcccagatttgaagaatatacttgtgatggtgatatcacgacaaatgaaaatgctgctgcctcaagtgaagctgccacaggtaagtgtaaaggagattatttagaggcttctatttccttctctcctacaaatatttcatttcagtgatgtttcttatagagtgtttgtgttaccttaatttaggcgatcagagaattccagaagtttcattggaggttcgtgggtgccgtaaagagactcagcacatgtcattgactgataaggcagcagtagatgaagaaatgccagcttcagtttatttggatatgcaaaactctttgtgtctgtcaaatggaacaccaatattggaggaatacacctgtgatggttatattccaccaaatgaagatgctgctgcttcaaatgatgacaatgaaagatacatagctacatcaaaagaggtgaataacatggaaaaaatagtcttggttacgggcagcccttcaagagaaagaggccatgatgacattgctactgatgttgcagtcagtgaattggtacacagtactccagcaacaggcacatgtaattatgttagaacagatatttaatcctgcaaatacattattttgttggtgtttctaatagtattttttaaaataatttagatcgtaaaaagactcctgtggcttctttgaagagtcaaggttcctggaaggaaaatcagcccgtagcttcaaataagaaggtattggatcaattaatgtaaacatcagcttattcaggtgtgcaagtcttgattgtggtccacaggagcttaagcttgttagctaataggcatcgtgcagtattttatgttgttattagtgactttgctgtttactcatgatgacatgttcatatttagcgtatttcagcctattgtcctatttgtgttactcatttatgtatatgcatatagatgaagttgattgatccgtgtcttggaaagcagcatgtaggccggccaaagaagcgaatatctccacatgcaaagtgtcaaagtgctacaagatctccagggaccaggaacccagcgtcatatgtaagtgcacttttttatctgttgtcataaactttagaaagtccatgattgcaagcagtagtgttttaacttgatgatgggatatagatctataacaatctcatttggtttgtggaattggactgtatgaattgtattggggatccaatttgattccatgtggagtcagaaatgggttccaatataattattttgtttgcataggtaaggaactgtggaaaacaaaatttgaccaaataaattctagctggtaatatgttacttgctccgagaaatccaattgcacagagaacacaactcgttgctttgctgctagacctttgacatgagaagccctatcacagattcacagccatgtcatgagcagtagcacacaagcacaggagggtggagccatacactcttttccccacgtgctcagccatactaactattggcctgtgttggctcacttgtcagcgatgacctggcctcattccactatctccattgtctaccagacagatttgtttgcctagcctgttgccggatccatgtcctattcagctaaccagatggggcctaagtattttgcatttttgcttgagatcgagctattgcaaaagtgatcatgcttcaagtacagattctgatgtttttggaacaatttatcttgacccagtagcatgagactgatattttttatatgtatttattgcaatgttcctgaatcacattaggtcctttggtctccgcttactcgtgataaggccacatcgttgtctatgtccacacctgaagatctcgaacttaaaagatccagatcaggcatgtctttgtttttttttttttggcaacaatctttggttgttttgactctgctgcttcattccaaatactcatcgtactaaagaaatggaaattttatttcaggtcgcgtgattgtgcccaaattggataattggtgccaaaccattgtctatggaagggtttgtttgttttctctatttgttatgttgttcaatcttagctactttgtgttataagaacacatatactatgctatgcttatatcttaactgaaagggtaataaaggattaaaggtatagttaatacatggtatcttaaatttagcaaatgaacattccattgcactaagagacaatgtgaaatactgtgatttgctaaatgccactgaatttgtgcacatttctagatcccttaattcagtatgtgatagaggtaattatatgactaacataaatttatcacagcggcaagaatgaaatatgataattcacttctatacagtgcagtttgtgaagtagaagtataaaccaagtgtcaaagttggttagtcaagatgcctgttctattatatgcatttcaaagttttgttagcctccagcgatgtattactgtcatggtattttttattttgcaatgggagacaatcccgaagaaagttcagataaatggtgaattatcatgacattctgattttgagcaatgtttttgtttccaaggcatttcctgtgcttagatctcatgaagtgccttgttcttgcaggatggtttgatcgcagctgtcattggtctagattcgccagcactgcccaaatgtttgtttggaatctctcatttgtccattctgttgtccttaagagcatgctttccatgttttgagagaattgcttaaagaaagtcaggtgatgccctatctagtttcatgtcatgcaacttgtgtcctttgctctgcagggagtgaatcaaaaactgatcgaaggaagaaacgaaagactaaatgagcatcttccaggcgtaattgctacttggctactccaggcgaagaaaattggaactaacttaatgtgattactccaggccaagaaaaccagaactaacttaatgtggcgggttggtcgcttttaatagattttcagtagaggagcctctcctgttctctctaaaaatatgtgtgtgtgtgtgtgtgggggggggggggggg

SEQ ID NO: 46 - protein, 513 aa, Sorghum bicolor
MKPLPVPEAGSPHRRGMPPSLLSPSSRSAVPAAADGDHDAAVSEHACVTLSEWWLATAEGDDQKIAVAGTFERNQTVQEYSPAPIAKRHTSSVLETEEGTVLRLHGLHNVLRTYHNGYSAKVYSEFLNGFPDWWQSCKPCNPKLMNSHTECCSSNASNSGVDSTQFYLERYMQGRRLDSYGTYLISKFPDILASFLHNDAVFQKSSHLLNGKPRFEEYTCDGDITTNENAAASSEAATGDQRIPEVSLEVRGCRKETQHMSLTDKAAVDEEMPASVYLDMQNSLCLSNGTPILEEYTCDGYIPPNEDAAASNDDNERYIATSKEVNNMEKIVLVTGSPSRERGHDDIATDVAVSELVHSTPATGTYRKKTPVASLKSQGSWKENQPVASNKKMKLIDPCLGKQHVGRPKKRISPHAKCQSATRSPGTRNPASYVLWSPLTRDKATSLSMSTPEDLELKRSRSGRVIVPKLDNWCQTIVYGRDGLIAAVIGLDSPALPKWSESKTDRRKKRKTK

SEQ ID NO: 47 - DNA, 5678 bp, Helianthus annuus
  misc_feature (355)..(355): n is a, c, g, or t
ctcagggaccatttgtgcagtttactctaaagtttttaaacccgagtttgattttagcggttgcgagccctagttgttgagatcaggacctctgattccaatggcttcttgctcttacttccagaagactgtaagtttttcttcctaaattcttccttccttccttccttccttccttccttcattcctcacacatttccctctttcttgcaggtcactctgctagactggtggctaaccaaacccccaaccaacgatcactatcaaaccctaaccctaggggttgcaggcttcacttctcaacagtcagtccgccctctctctctctctctctctctctaaatatgttcatatntattctagttagttagttgtgaaattgaaatgtaatttgttgataggaaccggcctgctcgatgtttctcttctgcgcccatactcaagatcttcgatttatttgagttggagacagttgatggtgtatgcgtcattctccagggttttattaacaaacaacgcacccttgaaaatggattttcccctcaggtattcttccctatttttcatatgctcttccacacaaagtatttccttattttgttttaattaattgtatatttatatcaatcatccttttttcaaccctcagacattggttacaaccattaaatcatatatcatagatcacttttgtcaatattactaatagctgatctagaatttttgtgtataacaagatagctggataactaagaatctcaatgacgtttaatcatattacctaggtggaccatttaggtcacataatagttttttggccattttgcacatgttgacccgatattttttttttcgcttaatccgagtatgaatttatattattcttaaacaatactctagttttcttgaataacatagtttaggaaattttatgcagtaaaaatacactttaggtgactttgacccatttgacttgggttagaattttttgtttacacatttgagccgggggtctcactggaagcagcctctctattcttacggggtagaggtaagactgtctacatcttaccctcctcagaccctaccttagctttgctattggtgggatttaccgagtatgatgatgatttgaaccattacaaataaaaacataacctgagttagcccattcgtaggtaaatggttgaaatgtcgatctctagttctattaaaatccaacattgaccttttctcacacttttcccttttgtaatatgatatttgttacatgtgcaggtgtttgatcatttttttatcgggttccccccttactggaaagaatactgtcccaagatagaatctgctgccaaatgtgtcacaggaggtaaatacgaatcgctttaagtagtagtttactaaaaacccaacgggtcaacaatccaagggtaacatgcttattcatgttattctcgtctggccacggattcatatcccatatggctagtttagaagaaagttttatcgttcattgaagatttaattggttggctgtttgtttacatcttaatgaggctcttaatggttcagacgtcttactggtttagcacttaatggttcatactgtttgtttcgcgagcaaatgtctgaatggttcagacatttgtctctgaatgatcaagcattatacaaagtctgaatgattaagacctctaatcttaattggtcagacatttgactctgaacggttaagtattatactacctcttaatggttcaaacctcttactggttcagcacttaatgattcaaacctcttactgattcagcacttaaccattcagaagttgccaaacagccctttagacgggtgtaattatgtacaaaattttgcgagtatggaacatgcatttctcactttctccatatgataattatcttcagttcaggaagaagactccattgaaggatatggtaaaccacataattctgatagctacactgtggatatgggagttcaagattgcaaagacgtaatgttgaacaataaaagtagtaatccatcctcggttgaaatttcacatgtatagttctcatctctccattggcatttatattttcaattcgttgcaatcttttgaaataaaaaacccaaaaagaaatttattcttagtacgtttgtctcatggttgcctaaacaaaatgttttcaagtgtctcttacaccattacaatggcaagcatgagtggttgagccttgaaacctgtgataatattaacccgtaactctttctggatcttggggtcacatacaaccctctaaaaatgaatcttatttctttaaacaggagcatataactgaaagatctcctacgacagcagaatttaaggatgatccaagtctcgagatgaatcccgttgactcatccacaccatcaaagtgttttggggttcctagcaggcgcgtgactagatctatgaaaaagccggatagcagtaaacatagttttctactatttaatggcattgatcctgggattttaggcagttctgagaatttaaacaagaaggctgtaaagatggaatcaaaatggaaacagattgaccaaaatggtgatgttactaaggataagagaaacaacgatgatactgttgtaagcagtgattcacatattaacataaggataagtgatttagaggatacacacgtcacacctaagtgttctgatccatcaagtgtgggtgtgatagatgtaaatgacgatgtgggaactaacatgaaaggctacagaaacaagaaaaaaaacagagttaacattccacagaaagaaggtatacctgcaacacatggaaccagttccaaagcagtcaagactcagaacaggtctaaaaccaaactactggttaaaaggaaactcgtaacaagtcctaaatcagctttttcaatgcgcaagaaggtaaatctacaaacaactctgattatacttgtttgttatggattaacaggttgttattgtataggaacgagatggaagtgcaaacatgttgtcgatagaatcattcagtgggaaaaaatctagatcaggttagagaagcaaccatatatataagtagttggatgtctaaagcataagataattaaatggtttattttatacagagtatatatgtgtgggcgctcggggggctaaaatgaaagtacactaattttaacgttaattttactaatttcgtgaaaaaaacgttagaagtggaggatggtaatcatgcatcatgtggtggcgttgatagccgaaagacaagggagtacatgcactaatcggcctttgtcttaattgctgccgagtgttatgtgccttatgtccaaggcttgatgcaaaactactatcgagccgggggtctcctggagtcagcctctctattcctacggggtagggctgtctacatcttaccctcgtcagaccctaccttagctttgcaattggtgggatttactgagtatgatgatgatgatgattttatacaaagtgaatttcaaattttgtccttttactttataccccttttcaggcggtgtcctttgtctttaaaattgacgagttttatacttcatgttttgaaatgttgcacgttatgtcctttaagcttaactcagttaattttttctgttaaatttgatcattcattactcaagggcatttttgtctttataccaattactttagaaacaacttaataaataaaacaaaaacaaatttaaaaaactaaaacactctcatatctcctctttctctcaatcaccactcccaaccagccatgacctactgccaacaccaccaccacccaccccttacaaccatccaccaccacccgaccatcgccaccaccggttcaccacccccagccgaccagcacaacacagctcacaccctctccccaatttcaaaccccacaaataaaaaacccccaatttcaaattcctaattcaaacccacctcaattattatctgaatcggaatcaaaatcagattttggacgatgttccagacttcaacttgatttaggggggtttgaattcaccgcaatcgaaccccacacagacttaacttcacctaaccataaacacatcaccccagccaccgtttgcaccacccacaacctccctctcatcccaaaactcttccctaacttgtccaaaacgatccctcctgtcctgtcggctgcaacacccaatttacaaacccgaagctgattcagaaccctatccatctttccttagtgtaacacccaaaaccctatccatcttcgccaaaaccacctgattcagaaccgccactcagtcaatcaccgccggttgcccttttgttcagtttaaaccgtcagtcaatcgccgccggttgccctatcctcgtcgagctcctatcgtacaccgccgtcgtgtttgaatcaggtcctccgccgccggagcctttttgttcattttaaaccatcatgttccttcactgtttgaatgtttcaaatggttttcaacattcagtagagagagagggagggaggttgagagagagaggggggacagtaaagttaattttatgtctttttaattattttacacaattgtccttagattttaaatatttgtaaactaatccctgaaaagtgaaatgacaataataccttcatgtgcaactcacatgaccggatttaacagaaaaatctaacagggttcgggctaaaggacataacgtgcaagattttaaaacaccaagtacaagactcgtcaatttgaaaaataaaggacaccgcctgaaattcactataaagataaaggacaaaatttgaaattcactcttttatacaggaagagtggtcctaccgcctttggaattctggcgcaaccaaaagcttgtttatgatgaggtttgtgtttcttttttaaatttttgcctgtttttaacacctgttatatgactcagattactaaatgtgtgtgcgtcttaggaattatgttgtttaggattatttctgtatgtttataagttgcatttttcccagtaacatacatatatatagcttttccagaacctgaaatgttgacatgaaattgtttgatataccattgacccactaatttggtggtatgtttgagggccctgaagtagtaattgaattaaattaaagaaaatgaagtttggggaggaggaaaacatgaacagtttaagatattaagtcgtattaaccagataaatttgaatttttattttgaatgattatttggtgtgagaatattgtaaaaaaaagtaaaatagcaatatgtaaacttcattaattaataaggaagtaaaatagcaatatgtaaacttcattaattaacaaggtctctttttaaaattcgctaaagactccttaatgacgtgagtcgggtcgtatgtggggcaaggtattaccgaagtgttggaagaattttgttttagtgttatctagtatctcctaatctttgttatcttatgataatcatgcttgaaattgaacatgcgagtttcttagtgttattacaattttcaggatggagaggtgtgtggagtccaaggacctatgtaacaacaatgaagaagaggtagtttgcatgatttagcatataacgtagctagtttttggggtgcactaacgattgtttgaacttgacaccgattgagacggg

SEQ ID NO: 48 - protein, 420 aa, Helianthus annuus
MASCSYFQKTVTLLDWWLTKPPTNDHYQTLTLGVAGFTSQQNRPARCFSSAPILKIFDLFELETVDGVCVILQGFINKQRTLENGFSPQVFDHFFIGFPPYWKEYCPKIESAAKCVTGVQEEDSIEGYGKPHNSDSYTVDMGVQDCKDVMLNNKSSNPSSVEISHEHITERSPTTAEFKDDPSLEMNPVDSSTPSKCFGVPSRRVTRSMKKPDSSKHSFLLFNGIDPGILGSSENLNKKAVKMESKWKQIDQNGDVTKDKRNNDDTVVSSDSHINIRISDLEDTHVTPKCSDPSSVGVIDVNDDVGTNMKGYRNKKKNRVNIPQKEGIPATHGTSSKAVKTQNRSKTKLLVKRKLVTSPKSAFSMRKKERDGSANMLSIESFSGKKSRSGRVVLPPLEFWRNQKLVYDEDGEVCGVQGPM

SEQ ID NO: 49 - DNA, 672 bp, Artificial Sequence
  cDNA of Saccharomyces cerevisiae Scm3 protein
atgaaaaccaataagaaaatttctaaaagaagaagcctgaaaaatctccatggtgccttaaaaggactattgaaagagtccggcaagaagagtgagtccaaaatacgaaaacatagcgattgcaatcctgttcatcgagtatatccgccaaatatcgaaaaacggaagaccaaaaaggacgatggaatttcacggccaattgctgaaagaaatggtcatgtgtatataatgtccaaggaaaaccacatcatccctaaactaaccgatgatgaagtcatggagcgccacaaactagcagacgaaaacatgagaaaagtgtggtccaacatcataagcaaatacgagtctattgaagaacaaggagacctcgtagacctgaaaactggtgaaatagttgaagataatgggcatatcaagacgctgacggccaacaacagcacgaaagataaaagaacaaaatacacaagcgtgctcagagacattatcgacatcagcgacgaggaagatggtgataaaaatgatgaatatactctttgggctaacgatagcgaggcaagcgattcagaggtggatgctgacaacgacactgaagaggaaaaagacgagaaattgatagacgcagatttcaagaagtacgaggccaaactctcgaaaaggatattacgagattaa

SEQ ID NO: 50 - protein, 223 aa, Saccharomyces cerevisiae
MKTNKKISKRRSLKNLHGALKGLLKESGKKSESKIRKHSDCNPVHRVYPPNIEKRKTKKDDGISRPIAERNGHVYIMSKENHIIPKLTDDEVMERHKLADENMRKVWSNIISKYESIEEQGDLVDLKTGEIVEDNGHIKTLTANNSTKDKRTKYTSVLRDIIDISDEEDGDKNDEYTLWANDSEASDSEVDADNDTEEEKDEKLIDADFKKYEAKLSKRILRD

SEQ ID NO: 51 - DNA, 3123 bp, Artificial Sequence
  cDNA of Arabidopsis thaliana SAD2 protein
atggatctgcatagcctcgctttgatcctccgaaccgccgctcttagccctattcccgatgagcgtaaagtttctgagcagcaactcaatcagttggagcacacgccccagcatttggtgaggttattgcagatagccgtggatggaaattgtgacatggcagtgcgtcaaattgctagtattcagttcaaaaatttaattgctaagaactggtcacctgaggattgtggtcctgcagtaaggcagcagcagatatttgagagtgacaaagaattggtgagggacaatattctcgtctatgtcacccaagttccaaccttactcagatcacaacttggagagtctctcaagacaattatttatgctgactacccagaacaatggccacgtcttctggattgggtgaagtacaacttgcaaaatcaacaaatttatggggctttgtttgtgttgcggatactctctagaaaatatgagttcaagtcagatgaggagaggacaccggtttcccgcattgtggaggagacatttcctcaacttttgactatttttaatgggcttatccagataccaaatccgtctctggagatagcagaacttatgaagttgatatgcaaaatattttggtcatccatatatctggagcttccaaggcaattatttgatctaaacgtctttaatgcctggatggttctgttcttgagtgtttcggaacgtcctgttccagttgaaggtcagcctatggatccggaacttagaaaatcttggggctggtggaaggtcaagaagtggacagtgcacattttaaacaggctctatagtcggtttggagatccaaaacttcaaagtccagaaaacaaacctttcgcccaaatgtttcaaaagaattatgcaggcagaattttggaaggccacctaaatttcttgaacacaattcgtgttggaggctatcttcctgacagagttatcaatcttctccttcagtacttaagcaacagcatttcgaagaatagcatgtacaagttgcttctgcctcggctggatgttctgctttttgagattgttttccctctaatgtgctttaatgataatgatcaaaagctttgggaggaagaccctcatgaatatgtgaggaaaggttacaatatcattgaagacttgtacagtccgcgaacagcgtccatggattttgtaaatgagttggttcgaaaacgtgggaaagaaaaccttccgaagtttgttaagtttgtcgttgagatcttcttgagttatgaaaaagctactgtagaggagaagccttatcgccaaaaagatggtgcgatgctggctgttggagctctttgtgacaaattgaagcaaacggatccctataaatctcaactagagctaatgttggtgcaacatatttttcctgatttcaatagtccagttggacatcttagagcaaaggctgcatgggtagctgggcaatatgcacacatcaatttctcagatcagaacaactttcgtaaagcattgcacagtgttgtttcgggactgcgcgatcctgatctccctgttcgtgttgattcagtttttgcattgcgttcatttgttgaggcgtgcaaggatttaaatgagatacgtccaatcctccctcagttacttgatgaatttttcaaactcatgaatgaggtagagaatgaggaccttgtttttactttggagaccatcgtagataagtttggcgaagagatggctccttttgcttttggattatgtcaaaatctggcggctgcgttttggagatgtctaaacacgtcagaagccaatgacgactctgatgacatgggagctttagctgccgttggttgtttgcgtgccataagtacaatccttgaatctgttagcagtctcccgcagctgtttgttgagatagaaccaactatacttccaataatgcagaaaatgttgaccactgatggccaagaggtatttgaagaagttttggagattgcatcatacatgaccttttattcacctagcatttccttggatatatggagtctctggcctttaatggtggaagcattggttgattggggaatcgatttctttccaaatattttggttccaatggacaactttatatcaaggggaacggctcattttctcacttgcaaagagcccgactatcagcaaagtctatataacgttctttcaactcttatgaccgacagaaacatagaagacagtgaaattgagtctgctccaaagcttattgaagttgttttccagaattgcaaagggcaggtggatcagtgggttgaaccatatctgcgtctcactgttgaccggttacaacgggctgagacttcgtatgttaaatctcttcttatacaagtggtggcaaatatgctttactacaatccaggtttgacgcttggtgtattgcataacacaggacttgcttccaaagtctttgacctttggttccagatgttgcagcaaaagagaaaaagtggcctgccagcaaacttcaaaagggaacatgataaaaaggtttgctgcttgggtttgacttcattacttgctctccctgggggtcaattccccgatgaagcgctgcagcgtgttttcagggcaacacttgatcttctagttgcatataagaatcagctagctgaagcagctaaggagacagaagtagattatgaagaagaaatgaatggactccagagtagtgatgatgattatgatgatgatgggtctgatggggagatggatgatacagaggaaggagatgaagcacaaagtgttaaactgcagaagttagctgcacaggcaaaggccttccactatgatgatgatgatgacgacgattctgatgatgactttagtgatgaggacgagtttcagtcacccatcgatgaggtggatgcctttgtattctttgtcgatgcaatcagagttatgcaggcatcagatgcgcagaggtttcagaacctgaaccagtcactggacttcacttaccaggcgattgcaaacggaatagcacagcatgcagagctgagaagagtggagatcgagaaggagaagcaaaagaagttagcagctgcttctactcctgttactgctctatga

SEQ ID NO: 52 - protein, 1040 aa, Arabidopsis thaliana
MDLHSLALILRTAALSPIPDERKVSEQQLNQLEHTPQHLVRLLQIAVDGNCDMAVRQIASIQFKNLIAKNWSPEDCGPAVRQQQIFESDKELVRDNILVYVTQVPTLLRSQLGESLKTIIYADYPEQWPRLLDWVKYNLQNQQIYGALFVLRILSRKYEFKSDEERTPVSRIVEETFPQLLTIFNGLIQIPNPSLEIAELMKLICKIFWSSIYLELPRQLFDLNVFNAWMVLFLSVSERPVPVEGQPMDPELRKSWGWWKVKKWTVHILNRLYSRFGDPKLQSPENKPFAQMFQKNYAGRILEGHLNFLNTIRVGGYLPDRVINLLLQYLSNSISKNSMYKLLLPRLDVLLFEIVFPLMCFNDNDQKLWEEDPHEYVRKGYNIIEDLYSPRTASMDFVNELVRKRGKENLPKFVKFVVEIFLSYEKATVEEKPYRQKDGAMLAVGALCDKLKQTDPYKSQLELMLVQHIFPDFNSPVGHLRAKAAWVAGQYAHINFSDQNNFRKALHSVVSGLRDPDLPVRVDSVFALRSFVEACKDLNEIRPILPQLLDEFFKLMNEVENEDLVFTLETIVDKFGEEMAPFAFGLCQNLAAAFWRCLNTSEANDDSDDMGALAAVGCLRAISTILESVSSLPQLFVEIEPTILPIMQKMLTTDGQEVFEEVLEIASYMTFYSPSISLDIWSLWPLMVEALVDWGIDFFPNILVPMDNFISRGTAHFLTCKEPDYQQSLYNVLSTLMTDRNIEDSEIESAPKLIEVVFQNCKGQVDQWVEPYLRLTVDRLQRAETSYVKSLLIQVVANMLYYNPGLTLGVLHNTGLASKVFDLWFQMLQQKRKSGLPANFKREHDKKVCCLGLTSLLALPGGQFPDEALQRVFRATLDLLVAYKNQLAEAAKETEVDYEEEMNGLQSSDDDYDDDGSDGEMDDTEEGDEAQSVKLQKLAAQAKAFHYDDDDDDDSDDDFSDEDEFQSPIDEVDAFVFFVDAIRVMQASDAQRFQNLNQSLDFTYQAIANGIAQHAELRRVEIEKEKQKKLAAASTPVTAL

SEQ ID NO: 53 - protein, 1032 aa, Beta vulgaris
MDLPSLAVILRAALSPNPDERKAGEESLNQFQYTPQHTVRLLQIIVDGNCDMAVRQVASIHFKNFIAKNWSPHDPEEQSKIAPADKDMVRENILVFVAQVPTLLRVQLGECLKTIIHTDYPEQWPGLLHWVKHNLQDQQVYGALFVLRILARKYEFKSDDERTPVHLIVEETFPPLLNIFNKLVQIPNPSIDVAELIKLICKIFWSSIYLEIPKQLFEPNLFNAWMVLFLNILERPVPLEGQPADLEQRKSWGWWKVKKWTVHILNRLYTRFGDMRLQNPENKAFALMFQKSYAGKILDCHLNLLNVIRAGGYLPDRVINLILQYLSNSISKSSMYQLLQPRLDVLLFEIIFPLMCFNDNDQRLWDEDPHEYVRKGYDIIEDLYSPRTAAMDFVSELVRKRGKENLQKFIQFVVEILKRYDEIPVEHKPYRQKDGALLAIGALCDKLKQTAPYKSELERMLVQHVFSEFNSPAGHLRAKAAWVAGQYAHINFSDQDNFRKALHSVVAGLRDPELPVRVDSVFALRSFVEACKDLGEIRPILPQLLDEFFKLMNEVENEDLVFTLETIVDKFGEEMAPYAFGLCQNLAAAFWKCMSTAEADEEGDDPGGALAAVGCLRAISTILESVSRLPHLFAQIEPTLLPIMRRMLTTDGQEVFEEVLEIVSYMTFFSPTISLDMWSLWPLMMDALSDWAIDFFPNILVPLDNYVSRSTAHFLTCKDPDYQQSLWKMISSIMSDNNLEDGDIEPAPKLIEVVFQNCKGQVDHWVEPYLRITVERLRRAEKPYLKCLLIQVIANALYYNPSLALSILHQLGVATEIFNLWFQMIQQVKRSGARANFKREHDKKVCCLGLTSLLSLPADQLPVEALGRIFRVTLELLVAYKEQVAETAKEDEAEDDDEMDGFESDGEDEGDGSDKEMGVDEDEGDDADSNHLNRLAARAKAFRSTDEDDDDSEDDYSDDEELQSPIDEVDPFILFVDTVKVMQGSDPLRFQNLTQTLEFQYQALANGVAQHADQRRVEIEKEKLEKAAAAPS

SEQ ID NO: 54 - protein, 1039 aa, Brassica napus
MDLPSLALILRAAALSPNPDERKASEQQLNQLQHTPQHLVRLLQIVVDGNCDMAVRQIASIQFKNFIAKNWSPVDSGEQPRILQSDKELVRDNILVYVTHVPTLLRSQLGECLKTIIYSDYPEQWPRLLDWVKYNLQNQQIYGALFVLRILSRKYEFKSDEERTPVSRIVEETFPVLLTIFNGLIQIENPSLEIAEFMKLICKIFWSSIYLELPKQLADPNVFNAWMLLFLTVSERPVPVEGQPMDPELRKSWGWWKVKKWTVHILNRLYSRFGDPKLQTLENKPFAQMFQKTYAGRILEGHLNFLNTIRLGGYIPDRVTNLLLQYLSNSISKNSMYSLLLPRLDVLLFEIVFPLMCFNDSDQKLWEEDPHEYVRKGYNIIEDLYSPRTASMDFVNELVQKRGIENLPKFVQFVVGIFRSYDEAPAEHKPYRQKDGAMLAVGALCDKLKKTEPYKSELEHMLVQHIFPEFSSPAGHLRAKAAWVAGQYAHINFSDQNNFRKALHSVVSGMRDPDLPVRIDSVFALRSFVEACKDLNEIRPILPQLLDEFFKLMNEVENEDLVFTLETIVDKFGEEMAPFAFGLCQNLAAAFWRCLNTSEAGDDSDDMGALAAVGCLRAISTILESVSSLPQLFVEIEPTILPIMQKMLTTDGHDVFEEVLEIASYMTFYSPTISLDIWSLWPLLVEALVDWAIDFFPNILVPMDNFISRGTAHFLTCKEPDYQQSLFNVLSTLMTDRNIEDSDVESAPKLIEVVFQNCKGQVDQWVEPYLRLTVDRLQRAETSYLKSLLIQVVANMLYYNPGLTLGVLHNTGLGSTVFDLWFQMLQQKKKSGLPANFKREHDKKVCCLGLTSLLALPGGQFPDEALQRVFRATLDLLVAYKNQIAEAKEAEVDYENEMNGLDSDDEDDDGSDGEMGMDDTEDGDEAESMRLKKLAAQAKSYGYDDEDEDDDDSDDGSDDDDEFQSPIDEVDAFVFFVDAIRVMQASDAQRFQNLNQSLDFTYQAIANGIAQHAEVRRVEIEKEKQKKAAEAAGTPVPAI

SEQ ID NO: 55 - protein, 1037 aa, Brassica napus
MDLPSLALILRAAALSPNPDERKASEQQLNQLQHTPQHLVRLLQIVVDGNCDMAVRQIASIQFKNFIAKNWAPVDSGEQPRILQSDKELVRDNILVYVTQVPTLLRSQLGECLKTIIYSDYPEQWPRLLDWVKYNFQNQQIYGALFVLRILSRKYEFKSDEERTPVSRIVEETFPVLLTIFNGLIQIENPSLEIAEFMKLICKIFWSSIYLELPKQLTDPNVFNAWMLLFLTVSERPVPVEGQPMDPELRKSWGWWKVKKWTVHILNRLYSRFGDPKLQTLENKPFAQMFQKTYAGRILEGHLNFLTTIRLGGYIPDRVTNLLLQYLSNSISKNSMYSLLLPRLDVLLFEIVFPLMCFNDSDQKLWEEDPHEYVRKGYNIIEDLYSPRTASIDFVNELVQKRGKENLPKFVQFVVGIFRSYDEAPAEHKPYRQKDGAMLAVGALCDKLKKTEPYKSELEHMLVQHIFPEFSSPAGHLRAKAAWVAGQYARINFSDQNNFRKSLHSVVSGMRDPDLPVRIDSVFALRSFVEACKDLNEIRPILPQLLDEFFKLMNEVENEDLVFTLETIVDKFGEEMAPFAFGLCQNLAAAFWRCLNTSEAGDDSDDMGALAAVGCLRAISTILESVSSLPQLFVEIEPTILPIMQKMLTTDGHDVFEEVLEIASYMTFYSPTISLDIWSLWPLLVEALVDWAIDFFPNILVPMDNFISRGTAHFLTCKEPDYQQSLFNVLSTLMTDRNIEDSDVESAPKLIEVVFQNCKGQVDQWVEPYLRLTVDRLQRAETSYLKSLLIQVVANMLYYNPALTLGVLHNTGLGSTVFDLWFQMLQQKKKSGLAANFKREHDKKVCCLGLTSLLALPGGQFPDEALQRVFRATLDLLVAYKNQIAAAKEAEVDYENEMNGLDSDDEDDDGSDGEMGMDDTEDGDEAESMRLKKLAAQAKSYGYDDEDDDDSDDGSDDDDEFQSPIDEVDAFVFFVDAIRVMQASDAQRFQNLNQSLDFTYQAIANGIAQHAEVRRVEIEKEKQKKAAEAAGAPVPAI

SEQ ID NO: 56 - protein, 1040 aa, Brassica oleracea
MDLPSLALILRAAALSPNPDERKASEQQLNQLQHTPQHLVRLLQIVVDGNCDMAVRQIASIQFKNFIAKNWAPVDSGEQPRILQSDKELVRDNILVYVTQVPTLLRSQLGECLKTIIYADYPEQWPRLLDWVKYNLQNQQIYGALFVLRILSRKYEFKSDEERTPVSRIVEETFPVLLTIFNGLIQIENPSLEIAEFMKLICKIFWSSIYLELPKQLADPNVFNAWMLLFLTVSERPVPVEGQPMDPELRKSWGWWKVKKWTVHILNRLYSRFGDPKLQTLENKPFAQMFQKTYASRILEGHLNFLNTIRLGGYIPDRVTNLLLQYLSNSISKNSMYSLLLPRLDVLLFEIVFPLMCFNDSDQKLWEEDPHEYVRKGYNIIEDLYSPRTASMDFVNELVQKRGKENLPKFVQFVVGIFRSYDEAPAEHKPYRQKDGAMLAVGALCDKLKKTEPYKSELEHMLVQHIFPEFSSPAGHLRAKAAWVAGQYAHINFSDQNNFRKSLHSVVSGMRDPDLPVRIDSVFALRSFVEACKDLNEIRPILPQLLDEFFKLMNEVENEDLVFTLETIVDKFGEEMAPFAFGLCQNLAAAFWRCLNTSEAGDDSDDMGALAAVGCLRAISTILESVSSLPQLFVEIEPTILPIMQKMLTNDGHDVFEEVLEIASYMTFYSPTISLDIWSLWPLLVEALVDWAIDFFPNILVPMDNFISRGTAHFLTCKEPDYQQSLFNVLSTLMTDRNIEDSDVESAPKLIEVVFQNCKGQVDQWVEPYLRLTVDRLQRAETSYLKSLLIQVVANMLYYNPALTLGVLHNTGLGSTVFDLWFQMLQQKKKSGLAANFKREHDKKVCCLGLTSLLALPGGQFPDEALQRVFRATLDLLVAYKNQIAEAAKEAEVDYENEMNGLDSDDEDDDGSDGEMGMDDTEDGDEAESMRLKKLVAQAKSYGYDDEDEDDDDSDDGSDDDDEFQSPIDEVDAFVFFVDAIRVMQASDAQRFQNLNQSLDFTYQAIANGIAQHAEARRVEIEKEKQKKAAEAAGTPVPAI

SEQ ID NO: 57 - protein, 1039 aa, Capsicum annuum
MDLQNLAIILRGALSPNPDERKAAENSLNQFQHTPQHLVRLLQIIVDGSCDMAVRQVASIHFKNFVAKNWSPHDPAEQSKIMPSDKELVRQNILIFVAQVPSLLRVQLGECIKTMIHADYPEQWPTLLPWVKHNLQDQQVYGALFVLRILSRKYEFKSDEERAPVYHVVEETFPHLLNIFNRLVQIANPSIEVADLIKLICKIFWSSIYLEIPKQLFDPNVFNAWMVLFLNMLERPVPVEGQPADPELRKSWGWWKVKKWTIHILNRLYTRFGDLKLQNPDNKAFAQMFQKGYAGKILECHLNLLNVIRAGGYLPDRVINLILQYLSNSISKSNMYSLLQPRLDTVLFEIIFPLMCFSDNDQKLWEEDPHEYVRKGYDIIEDLYSPRTAAMDFVSELVRKRGKENLQKFILFIVEIFKRYEEAAPEYKPYRQKDGALLAIGALCDKLKQTEPYKSELERMLVQHVFPEFSSPVGHLRAKAAWVAGQYAHINFSDPNNFRKALHSVVNGMRDPDLPVRVDSVFALRSFVEACKDLNEVRPILPQLLDEFFKLMNEVENEDLVFTLETIVDKFGEEMAPYAIGLCQNLAAAFWKCINSSEAEEEADDAGALAAVGCLRAISTILESVNRLPHLFMHIEPTLLPIMRRMLTTDGQEVFEEVLEIVSYMTFFSPTISVDMWSLWPLMMEALADWAIDFFPNILVPLDNYISRSTAHFLTCKDPDYQQSLWNMISSVMGDKNLEDSDIESAPKLIQVVFQHCKGQVDQWVEPYIRITIERLRRTEKPYLKCLLVQVIADALFYNAPLTLNILQKLGVATEVFNLWFQMLGETKKSGTRANFKREQDKKVCCLGLTSLLPLPVDQLPGEALERVFKATLDLLVAYKDQVAEAAKEDEAEDDDDMNGLQTDEDDDEDDGSDKEMGDDAEEGDEADSTRLQKLAAQAKAFRSHDEDEDDDSDDDFSDDEEELQSPLDEVDPFIFFVETIKALQASDPLRFQSLTQTLDFHYQALANGVAQHAEQRKVEIEKEKMEKASAAAAAAAAS

SEQ ID NO: 58 - protein, 1029 aa, Cucumis sativus
MDLPSLAVVLQAVLSPNPDERKAAEQSLNQIQHTPQHLVRMLQIIVDNNCDLAVRQVASIHFKNYIAKNWSPVDPDEHQKISESDKDAVRKNILPFLSQVPSLLRVQLGECLKTIIHADYPEQWPSLLEWVKENLLASNVYGALFVLRILARKYEFKSDDDRTPVYRIVDETFPLLLNIFSRLVQIGDPSLEVAELIKFICKIFWSSIYMEIPKHLFDTHVFNAWMMLFLNILERPVPLEGQPADPELRKSWGWWKVKKWTVHILNRLYTRFGDLKLKNPESRAFAQAFQKNYAGKVMECHLNLLNVIRSGGYLPDRVTNLILQYLSNSISKNSMYSLLQPRLDSLLFEIIFPLMCFNDNDQKLWDEDPHEYVRKGYDIIEDLYSPRTASMDFVSELVRKRGKENLQKFIQFIVGIFNRYDEATIEFKPYRQKDGALLAIGALCDKLKQTEPYKSELERMLVQHVFPEFNSPVGHLRAKAAWVAGQYAHINFADQNNFRKALHSVVAGMRDPELPVRVDSVFALRSFVEACRDLNEIRPILPQLLDEFFKLMNEVENEDLVFTLETIVDKFGEEMAPYALGLCHNLAAAFWRCMNTAEADEEADDPGALAAVGCLRAISTILESVSRIPQLFVQIEPTLLPIMRRMLTTDGQEVFEEVLEIVSFMTFFSPTISMDMWSLWPLMMEALSEWAIDFFPNILVPLDNYVSRGTAHFLTCKAPDYQQSLWNMISSIMTDKNLEDGDIEPAPKLIQVVFQNCKGQVDQWIEPYLRITIDRLQRTEKSYLKCLLMQVISDALYYNASLSLNILQKLGVAADVFNLWFQMLQQVKKSGIRVNFRREQDKKVCCLGLTSLLALPADQLPGEALGRVFRATLDLLVAYKDQVAEAAKEEEVEEDDEMDGYPSDEDDDDGDGSDKEMGFDGEDGDEVDSIKLQKLAAQAKSFRPDDDDFDSDDDYSDDEEMQSPLDDVDPFIYFVDTIKAMQGLDPMRFQSLSQSLEFQYQALAHGVAQHAEQRRVEIEKEKLERAASS

SEQ ID NO: 59 - protein, 1026 aa, Glycine max
MDLAMILEAALSPQPDERKGAEQRLDEMQQAPQHPVSLFQIIVDSSNCNMPIRQVAAIHFKNLIAKNWTKISLDHKELLRNHILLFLPQLPPLLRSQLGECLKTIIHSDYPHHFPHLLDWIILNLQDHHHVHSSLFVLRILSRKYEFKSDDERTPIYRVVEDTFPLLLNIFNSFLQIPNPSIELADLIKLISKIFWSSIYLEVPKVLFDQNVFNAWMVLFLNVLERPVPLEGQPADPELRKSWGWWKVKKWTVHILNRLYTRFGDLKLHNLENKAFAQMFQKHYAGKILDCYLNLLNVIRVGGYLPDRVINLVLQYLSNSISKNSMYTALQPRLDVLLFEIVFPLMCFNDNDQKLWDEDPHEYVRKGYDIIEDLYSPKTASLDFVSELIRKRGKDNLHNFIQFTVEIFKRYDEAPAEYKPYRQKDGALLAIGALCDKLKQTEPYKSELEHMLVQHVFPEFSSPVGHLRAKAAWVAGQYAHINFSDQNNFRKALQCVVCGMRDPELPVRIDSVFALRSFVEACKDLNEIRPILPQLLDEFFKLMNEIENEDLVFTLETIVDKFGEEMAPYALGLCQNLAATFWRCMNTTEADDEADDPGALAAVGCLRAISTILESVSSLPQLFVQIEPALLPIMRRMLTTDGQEVFEEVLEIVSYMTFFSPTISLDMWSLWPLMMEALADWAIDFFSNILVPLDNYISRGTAHFLICKEPDYQQSLWNMISSVMGDKNMEDNDIEPAPKLIEVVFLNCKGQVDQWVEPYLRITVERLHRTEKSHLKCLLMQVIADALYYNAPLTLSILQKLGVTSEIFTLWFNMLQGVKKNGVRANFKREHDKKVCCLGLTSLLALPAGQLPGDALGQVFRATLDLLVAYKDQVAEAAKEEEAEDDDDDDMDDFQTDDEDEDDNGSDKEMGIDAEDGDEADSSKFTKLAEQTKTFRPNDEDDDDSDDDFSDDEELHSPIDEVDPFVFFVDTMKVMQSSDPLGFQNLTQTLEFSYQALANGVAQHAELRRGETEKEKLEKSSATIDS

SEQ ID NO: 60 - protein, 1027 aa, Helianthus annuus
MDLSNLVIVLKAALSPNPAERKAAEDSLNQYQYTPQHLVRLLQIVVDGNCDMAVRQAASIHFKNFIAKNWAPLDPEEQCKILPSDKDVVRQNILVFVAQLPPLLRAQLGECLKTIIHADYPEQWPGLLHWVTHNLQDQQVYGALFVLRILARKYEFKSDEERIPVYHVVEETFPHLLNIFSRLVQIANPSIEVADLIKLICKIFWSSIYLEIPKKLFDPNVFNAWMALFLNMLERPVPLEGQPIDPELRKSWGWWKVKKWTVHILNRLYTRFGDLKLQSPENKAFAQHFQKNYAGKILECHLNLLNAIRTGDYLPDRVTNLILQYLSNSISKTALYNLLQPRLDVVLFEIIFPLMCFNDNDQTLWEEDPHEYVRKGYDIIEDLYSPRTAAMDFVSELVRKRGKENLQKFILFIVEIFKRYEAASIEFKPYRQKDGALLAIGALCDKLKHTEPYKSELEPMLVQHVFPEFASPVGHLRAKAAWVAGQYAHINFSDSNNFRKALQSVVAGMRDPELPVRVDSVFALRSFVESCKDLSEIRPILPQLLDDFFKLMNEVENEDLVFTLETIVDKFGEEMAPYALGLCQNLAAAFWKCINTSEGDEEADDPGALAAAGCLRAISTILESVSRLPHLFAHTEPTLLPIMRRMLTTDGQDVFEEVLEIVSYLTFFSPTISMEMWSLWPLLLEALADWAIDFFPNILVPLDNYISRGTVHYLTCKEPDYQQSLWNVLSNIMSDKNLEDNDIEPAPKLIEVVFQNCKGQVDHWVEPYIRLTVERLRFTERPYLKCLLVQVIANALYYNPSLTLNILQKLGVATEVFNLWFQMLQQTKKSGARANFKREHDKKVCCLGLTSLLTLPSDQLPGEALERVFKATLELLVAYKDQVAEAAKDEPEDDDDMDDGLETDDDEDDMGDDAEDGDDADSLRLQRLAAQAKSFRPADDDDDDSDDEFSDDDELQSPIDEVDPFIFFMDIAKAMQASDQVRFQNLSQTLDFRYQALATGVAQHADQRRVEIEKEKLDKANAAAAAS

SEQ ID NO: 61 - protein, 1033 aa, Helianthus annuus
MDLPNLAIVLQAALSPNPAERKAAEDSLNQYQYTPQHLVRLLQITVDGNCDIAVRQAASIHFKNFIAKNWSPLDPDEQSKILPSDKDLVRQNILLFVAQVPPLLRAQLGECLKTIIHADYPEQWPGLLHWVTHNLQDQQQVYGALFVLRILARKYEFKSDEERIPVYLVVEQTFPHLLNIFGNLVQIGNPSIEVADLVKLICKIFWSSIYLEIPKKLFDPNVFNAWMVLFLNMLERPVPLEGQPADPELRKSWGWWKVKKWTVHILNRLYTRFGDLKLQNPENKAFAQHFQKNYAGKILECHLNLLNAVRTGDYLPDRVTNLILQYLSNSISKTTMYNQLQPRLDVVLFEIIFPLMCFNDNDQTLWEEDPHEYVRKGYDIIEDLYSPRTAAMDFVSELVRKRGKENLQKFILFIVEIFKRYEAAPVELKPYRQKDGALLAIGALCDKLKQTEPYKSELEPMLVQHVFPEFVSPVGHLRAKAAWVAGQYAHVNFSDPNNFRKALQCVVAGMRDPELPVRVDSVFALRSFVESCKDLGEIRPILPQLLDDFFKLMDEVENEDLVFTLETIVDKFGEEMAPYALGLCQSLAAAFWKCINTCEGDEEADDPGALAAAGCLRAISTILESVSRLPHIFAHIEPTLLPIMRRMLTTDGQDVFEEVLEIVSYMTFFSPTISMDMWSLWPLLIEALADWAIDFFPNILVPLDNYISRSTVHYLTCKEPDYQQSLWNMLSNIMSDKNLEDNDIEPAPKLIEVVFQNCKGQVDHWVEPYIRLTVERLRVAERPYLKCLLVQVIADALYYNASLTLNVLQKLGVATEVFNLWFQMLQQTKRSGGRANFKREHDKKVCCLGLTSLLTLSADQLPGEALERVFKATLELLVAYKDQVAEAAKEGSEDDDGMNDDLETDDDEDDGSDKDMGVDAEDGDEADSLRLQRLAAQAKSFRPADDDDDDDDDDFSDDEDLQSPIDEVDPFVFFMDTAKALQASDQIRFQNLSQTLDFHYQALASGISQHADQRRVEIEKEKLEKASASAPAS

SEQ ID NO: 62 - protein, 1026 aa, Helianthus annuus
MDLQSLAIVLQAALTPNPAERKAAEDTLTKFQYTPQHLVRLLQIIVDGNCDLAVRQVASIHFKNFIAENWSPHDPDEQSKILPSDKDLVRQNILVFVAQVPALLRAQLGECLKTIIHADYPEQWPALLQWVTHNLQDQQVYAALFVLRILSRKYEFKSDEERTPIHHVVEETFPHLLNIFSRLVQIGNPSIEIAGLIKLICKIYWSSIYLEIPKKLYDPNVFDAWMVLFLNILERPVPLEGQPTDPELRKSWGWWKVKKWTAHILNRLYTRFGDLKLQNPESKAFAQHFQKNYAAKILECHLNLLNAVRVGGYLPERVANLSLQYLSNSLAKSTTYSLLQTRLDVVLFEIIFPLMCFNDDDQKLWEEDPHEYVRKGYDIIEDLYSSRTAAMDFVTELVRKRGKENLQKFILFIVEIFKRYEQAPIEIKPYRQKDGALLAIGTLCDKLKQTEPYKSELEPMLVQHVFPEFTSPMGHIRAKAAWVAGQYAHINFSDPNNFRKALQSVVAGMRDPELPVRVDSVFALRSFVEACTDLGEIRPILPQLLDEFFKLMNEVENEDLVFTLETIVDKFGEEMAPYAVGLCQNLAATFWKCMNTEADDEADDPGALAAVGCLRAISTILESVSKLPHLFAHVEPTLLPIMRRMLTTDGQEVFEEVLEIASYMTFFSPTISMDMWSLWPLLMEALAEWAIDFFSNILVPLDNYISRSTVHYLTCKEPDYQQSLWNMLSNIMNDKNLEDNDIDPAPKLIAVVLQNCRGQVDQWVEPYIRITVERLHRTERPFLKCLLMQVIADALYYNPSLTLNILQKLGVATEIFNLWFQMLQQTKKNGVRVNFKRENDKKICCLGLTSLLSLSADQLPLEALERVFKATLDLLVAYKDQLTEAEKEAPEDDDDDDDDDMNDSLQTDDDDYDDDDEDGDEANSSRLQKLGAQERVFRSTDDYDDDSDDDFSDDEDLQSPIDDVDPFILFVDTVKVMQASDPTRFQNLSQTLDFRYQALANGVAQHADQRRAVIEKEKLEKASVAS

SEQ ID NO: 63 - protein, 1036 aa, Lycopersicon esculentum
MDFQNLAVILAGALSPNPDERKAAENSLNQFQHTPQHLVRLLQIIVDGSCDMAVRQVASIHFKNFVAKNWCPHDPAEQSKIMPSDKELVRQNILIFIAQVPSLLRVQLGECIKTMIHADYPEQWPTLLPWIKHNLQDQQVYGALFVLRILSRKYEFKSDEERTPVYHVVEETFPHLLNIFNKLVQITNPSIEVADLIKLICKIFWSSIYLEIPKQLFDPNVFNAWMVLFLNMLERPVPVEGQPADPELRKSWGWWKVKKWTVHILNRLYTRFGDLKLQNPDNKAFAQMFQKGYAGKILECHLNLLNVIRAGGYLPDRVINLILQYLSNSISKSNMYSLLQPRLDIVLFEIIFPLMCFSDNDQKLWEEDPHEYVRKGYDIIEDLYSPRTAAMDFVSELVRKRGKENLHKFLFFIVEIFKRYQEAAPEYKPYRQKDGALLAIGALCDKLKQTEPYKSELEHMLVQHVFPEFSSPVGHLRAKAAWVAGQYAHINFADPNNFRNALHSVVTGMRDPDLPVRVDSVFALRSFIEACKDLNEIRPILPQLLDEFFKLMNEVENEDLVFTLETIVDKFGEEMAPYALGLCQNLAAAFWKCMNSAEAEEEADDPGALAAVGCLRAISTILESVSRLPHLFIHIEPTLLPIMRRMLTTDGQEVFEEVLEIVSYMTFFSPTISMDMWTLWPLMMEALADWAIDFFPNILVPLDNYISKSTAHFLTCKDPDYQQSLWNMISSVMGDKNLEDGDIESAPKLIQVVFEHCKGQVDHWVEPYIRLTVERLRRAEKSHLKCLLVQVIADALYYNAPLTWNILQKLGLALEVFNLWFLMLGQTKKSGKRVNFKREHDKKVCCLGLTSLLPLPVDQFPREALDRVFKATLDLLIAYKDQVAEAAKEDEAEDDDDMNGLQTDEDDDEDDESDKEMGDDAEEGDEADSTRLQKLAAQAKAFRSHDEDDDDDDSDDDFSDDDEEMQSPLDEVDPFIFFVETIKAMQASDPVKFQSLTQTLDFRYQALANGVAVHAEERRAEIEKEKLEKASAVVAS

SEQ ID NO: 64 - protein, 1037 aa, Sorghum bicolor
MDLPSLAVVLRAALSHVPEERKAAEESLNQFQYTPQHLVRLLQIIVDGSCDMAVRQVASIHFKNFIAKNWSPNDPDESPKVLESDKAMVRENILGFIVQVPPLLRAQLGESIKTIIHSDYPEQWPSLLHWVSHNLDLQNQIFGALYVLRVLARKYEFKSEDERIPLYRIVEETFPRLLSIFSKLVQIVNPPIEVADLIKLICKIFWSSIYLEIPKQLFDPNVFNAWMVLFINLLERPVPVEGQPIDPEIRKSWAWWKVKKWTIHILNRLYTRFGDLKLQKPESKAFAQMFQKTYAGKILACHMQLLNAIRSGDYLPDRVVNLVLQYLTNSVTKNSMYQMMQPQIDILLFEIIFPLMCFNDNDQKLWEEDPHEYVRKGYDIIEDLYSPRTAAMDFVSELVRKRGKSNLQKFIHFIVDIFRRYDEASADLKPYRQKDGALLAIGTLCDKLKQTDPYKSELERMLVQHVFPEFTSRVGHLRAKAAWVAGQYAHINFSDPNNFRQAMHCIVSGMRDPDLPVRVDSVFALRSFVEACKDLNEIRPILPQLLDEFFKLMNEVENEDLVFTLETIVDKFGEEMAPYALGLCQNLAAAFWRCMASSETDDEADDSGALAAVGCLRAISTILESISSLPHLFMQIEPTLLPIMRRMLTSDGQDVYEEVLEIVSYMTFFSPTISLDMWSLWPLMMEALNDWAIDFFENILVPLDNYISRGTDHFLECKDPDYQQSLWNALQSIMMDENMEDSDIEPAPKLIEVVFQNCKGNVDQWVEHYLRITIERLRRAKKPYLKCLLVQVIANALYYNPALTLETLNKLGVAADIFNHWFAMLQQVKKSGARVNFKREHDKKVCCLGLTSLIALPADKIPAEALDRIFKATLGLFFPSKLCLCFFSPGEYTEAKKQNEEGADDMDGFDADEEDDDEVDSDKEMGLDDEDGDEVSSLQLQKLAAEARGFQPADEDDDSDDDFSDDEELHSPIDEVDPFIFFVETVQGLQASDPARFQNLMQTLDFRYQALASGIAQHAEERKIEIEKEKSEKANAQ

SEQ ID NO: 65 - protein, 1027 aa, Zea mays
MDLPSLAVVLRAALSHVPEERKAAEESLNQFQYTPQHLVRLLQIIVDGSCDMAVRQVASIHFKNFIAKNWSPNDPDESPKVLESDKAMVRENILGFIVQVPPLLRAQLGESIKTIVHSDYPEQWPSLLHWVSHNLDLQNQIFGALYVLRVLARKYEFKSEDERIPLFHIVEETFPRLLSIFSKLVQIVNPPIEVADLIKLICKIFWSSIYLEIPKQLFDPNVFNAWMVLFINLLERPVPVEGQPIDPEIRKSWAWWKVKKWTIHILNRLYTRFGDLKLQKPESKAFAQMFQKTYSGKILACHMQLLNAIRGGDYLPDRVINLVLQYLTNSVTKNSMYQMMQPQIDILLFEIIFPLMCFNDNDQKLWDEDPHEYVRKGYDIIEDLYSPRTAAMDFVSELVRKRGKNNLQKFIHFIVDIFRRYDEASADLKPYRQKDGALLSIGTLCDKLKQTDPYKSELETMLVRHVFPEFSSRVGHLRAKAAWVAGQYAHINFSDPNNFRQAMHCIVSGMHDPDLPVRVDSVFALRSFVEACKDLNEIRPILPQLLDEFFKLMNEVENEDLVFTLETIVDKFGEEMAPYALGLCQNLAAAFWRCMASSEADDEADDSGALAAVGCLRAISTILESISSLPHLFTQIEPTLLPIMRRMLTSDGQDVYEEVLEIVSYMSFYSPTISLDMWTLWPVMMEALNDWAIDFFENILVPLDNYISRGTDHFLACKDPDYQQSLWKALQSIMMDENMEDSDIEPAPKLIEVVFQNCKGNVDHWVEHYLRITIERLRRAHKPYLKCLLVQVIANALYYNPALTLEILNKLGVAADIFNHWFAMLRQVKKSGARVNFKREHDKKVCCLGLTSLIALPAAKIPADALDRIFKATLELLVAYKDQVAEAKKQNEEAADDMDGFDADEEDDDEVDSDKEMGLDDEDGDEVSSLQLQKLAAEARGFQPADDDDSDDDFSDDELHSPIDEVDPFIFFVETVQGLQASDPARFQNLMHTLDFSYQALASGIAQHAEQRKNEIEKEKSEKANAQ

SEQ ID NO: 66 - protein, 1035 aa, Nicotiana tabacum
MDLQNLAIVLRGALSPNPDERKAAEESLNQFQYTPQHLVRLLQIIVDGSCDMAVRQVASIHFKNFVAKNWSPHDPAEQSKILLSDKELVRQNILIFIAQVPSLLRVQLGECLKTMIHADYPEQWPTLLPWVKHNLQDQQVYGALFVLRILSRKYEFKSDEERTPVYHIVEETFPQLLNIFNRLAQIANPSIEVADLIKLICKIFWSSIYLEIPKQLFDPNMFNAWMVLFLNMLERPVPLEGQPADPELRKSWGWWKVKKWTVHILNRLYTRFGDLKLQNPDNKAFAQMFQKGYAGKILECHLNLLNVIRAGGYLPDRVINLILQYLSNSISKSNMYSLLQPRLDIVLFEIIFPLMCFSDNDQKLWEEDPHEYVRKGYDIIEDLYSPRTAAMDFVSELVRKRGKENLQKFILFIVEIFKRYEEAAPEYKPYRQKDGALLAIGALCDKLKQTEPYKSELERMLVQHVFPEFSSPVGHLRAKAAWVAGQYAHVNFSDPNNFRKALHSVVAGMRDPDLPVRVDSVFALRSFVEACRDLDEIRPIIPQLLDEFFKLMNEVENEDLVFTLETIVDKFGEEMAPYALGLCQNLAAAFWKCINTSEADEEGDDPGALAAVGCLRAISTILESVSRLPHLFIHIEPTLLPIMRRMLTTDGQEVFEEVLEIVSYMTFFSPTISMEMWSLWPLMMEALADWAIDFFPNILVPLDNYISRSTAHFLTCKDPDYQKSLWNMISTVMGDKNLEDGDIEPAPKLIQVVFQHCKGQVDHWVEPYIRITIERLRRAEKPYLKCLLMQVIADALYYNASLTLNILQKLGIATEVFNLWFQMLGQKKKSGARVNFKREHDKKVCCLGLTSLLPLPVDQLPGEALERVFKASLELLVAYKDQVAEAAKEEEVEDDDDMHGLQTDEDDDEDDGSDKEMGVDAEEGDEADSTRLQKLAAQAKAFRSHDSDEDDDSDDDFSDDDEELQSPLDEVDPFIFFVETIKAMQASDPLRFQSLTQTLDFHYQALANGVAQHAEQRRVEIEKEKMEKASATAAS

SEQ ID NO: 67 - protein, 1030 aa, Oryza sativa
MDLQSLAVVLRAALSHVPEERKAAEESLNQFQYAPQHLVRLLQIIVDGSCDMAVRQVASIHFKNFVAKNWSPNDPEESQKISESDKLMVRENILGFIVQVPPLLRAQLGESIKTIIHSDYPEQWPVLLHWVTHNLESENQIFGALYVLRVLSRKYEFKSEEERIPLYHIVEETFPRLLSIFSKLVQIVNPPIEVADLIKLICKIFWSSIYLEIPKQLFDPNVFNAWMSLFINLLERPVPVEGQPLDPDVRKSWGWWKVKKWTIHILNRLYTRFGDMKLQKPESKAFAQMFQKNYAGRILGCHLQILNAVRTGDYLPDRVINLVLQYLTNSVTKNSMYQLMQPQIDIILFEIIFPLMCFNDNDQKLWDEDPHEYVRKGYDIIEDLYSPRTAAMDFVSELVRKRGKSNLQKFIHFIVDIFRRYDEASIEIKPYRQKDGALLAIGTLCDKLKQTDPYKAELERMLVQHVFPEFNSHVGHLRAKAAWVAGQYAHISFSDQDNFRKAMHCIVSGMRDPDLPVRVDSVFALRSFVEACKDLNEIRPILPQLLDEFFKLMNEVENEDLVFTLETIVDKFGEEMAPYALGLCQNLAAAFWRCMASQEADDEADDSGALAAVGCLRAISTILESVSSLPHLFIQIEPTLLPIMRRMLTSDGQDVYEEVLEIVSYMTFFSPSISLDMWSLWPLMMEALNDWAIDFFENILVPLDNYVSRGSDHFLACKNPDYQQSLWSALSSIMMDQNMEDSDIEPAPKLIEVVFQNCKGNVDQWVEPYLSLTIDRLRRAHKPYLKCLLVQVIANAFYYNPSLTLATLHKLGAVTEIFNIWFGMLEQVKKSGVRANFKREHDKKVCCLGLTSLISLPADHIPGEALNRIFKATLDLLVAYKEQVAESKKQDDADGDDMDGFDADEDEDDDEVESDKEMGLDEEDADEVNSLHLQKLAAEARGFQPADEYDDSDDDFSDDEELQSPIDEVDPFILFVETVQGLQASDPIRFQSLMQTLDFRYQALASGIAQHAEERRVEIEKEKLEKANAQ

SEQ ID NO: 68 - protein, 1030 aa, Oryza sativa
MDLQSLAVVLRAALSHVPEERKAAEESLNQFQYAPQHLVRLLQIIVDGSCDMAVRQVASIHFKNFVAKNWSPNDPEESQKISESDKLMVRENILGFIVQVPPLLRAQLGESIKTIIHSDYPEQWPGLLHWVTHNLESENQIFGALYVLRVLSRKYEFKSEDERIPLYHIVEETFPRLLSIFSKLVQIVNPPIEVADLIKLICKIFWSSIYLEIPKQLFDPNVFNAWMSLFINLLERPVPVEGQPLDPDVRKSWGWWKVKKWTIHILNRLYTRFGDMKLQKPESKAFAQMFQKNYAGRILGCHLQILNAVRTGDYLPDRVINLVLQYLTNSVTKNSMYQLMQPQIDIILFEIIFPLMCFNDNDQKLWDEDPHEYVRKGYDIIEDLYSPRTAAMDFVSELVRKRGKSNLQKFIHFIVDIFRRYDEASIEIKPYRQKDGALLAIGTLCDKLKQTDPYKAELERMLVQHVFPEFNSHVGHLRAKAAWVAGQYAHISFSDQDNFRKAMHCIVSGMRDPDLPVRVDSVFALRSFVEACKDLNEIRPILPQLLDEFFKLMNEVENEDLVFTLETIVDKFGEEMAPYALGLCQNLAAAFWRCMASQEADDEADDSGALAAVGCLRAISTILESVSSLPHLFIQIEPTLLPIMRRMLTSDGQDVYEEVLEIVSYMTFFSPSISLDMWSLWPLMMEALNDWAIDFFENILVPLDNYVSRGSDHFLACKNPDYQQSLWSALSSIMMDQNMEDSDIEPAPKLIEVVFQNCKGNVDQWVEPYLSLTIDRLRRAHKPYLKCLLVQVIANAFYYNPSLTLATLHKLGAVTEIFNIWFGMLEQVKKSGVRANFKREHDKKVCCLGLTSLISLPADHIPGEALNRIFKATLDLLVAYKEQVAESKKQDDADGDDMDGFDADEDEDDDEVESDKEMGLDEEDADEVNSLHLQKLAAEARGFQPADEYDESDDDFSDDEELQSPIDEVDPFILFVETVQGLQASDPIRFQSLMQTLDFRYQALASGIAQHAEERRVEIEKEKLEKANAQ

SEQ ID NO: 69 - protein, 1034 aa, Coffea arabica
MDLQNLAIILRGALSPNPDERKAAEESLNQFQYTPQHLVRLLQIIVDGNCDMAVRQVASIHFKNFIARNWAPHDPGEQSKILPSDKDVVRQNILNFIAQVPQLLRVQLGECLKTIIHADYPEQWPALLHWVKLNLQDQQVFGALFVLRILARKYEFKSDEERTPVYHIVDETFPPLLNIFNRLVQITNPSIEVADLIKLICKIFWSCIYLEIPKQLFDPNVFNAWMVLFLNVLERPVPLEGQPVDPEVRKSWGWWKVKKWTVHILNRLYTRFGDLKLQNPDNKVFAQMFQKNYAGKILECHLNLLNVIRVGGYLPDRVINLILQYLSNSISRSNMYNLLQPRLDVVLFEIIFPLMCFSDADQKLWDEDPHEYVRKGYDIIEDLYSPRTAAMDFVSELVRKRGKENLQKFILFIVEIFNRYNEAVPEYKPFRQKDGALLAIGALCDKLKQTEPYKSELERMLVQHVFPEFSSPAGHLRAKAAWVAGQYAHVNFSDPNNFRNALRSVVAGMRDPELPVRVDSVFALRSFVEACKDLDEIRPILPQLLDEFFKLMNEVENEDLVFTLETIVDKFGEEMAPYALGLCQNLAAAFWKCLNTAEADDEADDPGALAAVGCLRAISTILESVSSLPHLFVQIEPTLLPIMRRMLTTDGQEVFEEVLEIVSYMTFFSPTISMDMWSLWPLMMEALADWAIDFFPNILVPLDNYVSRSTVHFLTCKEPDYQQSLWHMISSIMADKNLEDGDIEPAPKLIQVVFQNCRGQVDHWVEPYIRIAVERLRRTEKPYLKCLLIEVIADALYYNASLTLSILHKLGVASDIFALWFQMLQQTKKNGVRANFKREHDKKVCCLGMTSLLSLPNDQFPAEALERVFKATLELLVAYKEQLAETEKEAAEDNDDMDGLPTDDEDDDGDVSDKEMGIDDEDGDEADSIKLQKLAAQAKAFRSTEDDEDDSDDDFSDDEELQSPIDDVDPFVFFVDTIKALQASDPLRFQNLTQTLDFHFQALANGVAQHAEQRRVEIEKEKMEKASSAAVGAS

SEQ ID NO: 70 - protein, 1031 aa, Triticum aestivum
MDLSNLTLVLRAALSHAPEERKAAEASLEQLQYTQQHLVRLLQIIVDGSCDMAVRQVASIHFKNFVSKAWSPIDPDETRKIPEGDKSMVRENILGFVTQLPPLLRAQLGESIKTLILADYPEQWPSLLHWVTHNMESQDQIFGALYVLRILSRKYEFKSEEERIPLYQIVEECFPRLLNIFSTLVQIVNPPIEVADLIKLICKIFWSSIYLEIPKQLFEPNIFNAWIVLFLNLLERPVPLEGQPSDPDARKAWGWWKVKKWITHILNRLYSRFADMKVHKPESKAFAQMFQKNYAGKILGCHLQLLNAIRTGGYLPDRVINLILQYLTNSLTKNSMYQLMQPQIDIILFEIIFPLMCFNDNDQMLWDEDPHEYVRKGYDIIEDLYSPRTAAMDFVSELVRKRGKGNLQKFIQFIVEIFMRYNEASIEVKPYRQKDGALLAIGTLCDRLKQTDPYKAELERMLVQHVFPEFSSHVGHLRAKAAWVAGQYAHITFSDQDNFRKAMHCVISGLRDPELPVRVDSVFALRSFVEACKDLDEIRPILPQLLDEFFKLMGEVENEDLVFTLETIVDRFGEEMAPYALGLCQSLAAAFWRCMASSEADDEVEDSGALAAVGCLRALSTILESISSLPHLFIQIEPTLLPILRRMLTSDGQDVYEEVLEIVSYLTFYSPTISLDMWSLWPLMMEALNDWAIDFFENILVPLDNYISRGTEHFVTCKDPDYQQCLWKGLSSVMTDQNMEDSDIVPAPKLIEVFFQNCKGQVDHWVEPYLRLTIDRLRRAEKPYLKSLLVQVIANVFYYNPSLTLAMLHKLGVATEIFNLWFVMLQQVKKSGKRVNFKREHDKKVCCLGLTSLIGLPANHIPAEALERIFKATLELLVAYKEQVAESKRQNAAAADDVDEFDADEEENEEDEDDGEMGVDDEDQDEVNSLNIQKLVQARGFQLHDEDDDDDDSDDDFSDDEELQTPIDEVDPFIFFVGTIQAVQASDPARFQSLMQTLDFHYQALANGVAQHAEERKVEIEKEKLEKANAQ

(19) United States
(12) Patent Application Publication
(10) Pub. No.: US 2023/0279418 A1
(43) Pub. Date: Sep. 7, 2023

(54) PLANT HAPLOID INDUCTION

(71) Applicant: KWS SAAT SE & Co. KGaA, Einbeck (DE)

(72) Inventors: Monika KLOIBER-MAITZ, Einbeck (DE); Christof BOLDUAN, Einbeck (DE); Alevtina RUBAN, Einbeck (DE); Milena OUZUNOVA, Gottingen (DE); Markus NIESSEN, Laatzen (DE)

(73) Assignee: KWS SAAT SE & Co. KGaA, Einbeck (DE)

(21) Appl. No.: 17/925,789

(22) PCT Filed: May 28, 2021
(86) PCT No.: PCT/EP2021/064425

§ 371 Date: Nov. 16, 2022

(30) Foreign Application Priority Data
May 29, 2020 (EP) 20177492.4

(51) Int. Cl. C12N 15/82; A01H 6/46; A01H 5/10; C07K 14/415

(52) U.S. Cl. CPC C12N 15/8262; A01H 6/4684; A01H 5/10; C07K 14/415

(57) ABSTRACT

The present invention relates to plants comprising a polynucleic acid encoding a mutated indeterminate gametophyte (ig) protein and a polynucleic acid encoding a mutated centromere or kinetochore protein, wherein said mutated centromere or kinetochore protein preferably is CENH3. The mutated ig and centromere or kinetochore proteins together result in haploid inducing activity, such as in particular paternal haploid inducing activity. The invention further relates to methods for generating such plants and uses thereof.

### FIELD OF THE INVENTION

The invention relates to the field of plant breeding, and in particular to the development of haploid inducers, as well as the use thereof for generating haploid plants and in doubled haploid technology.

### BACKGROUND OF THE INVENTION

The generation and use of haploids is one of the most powerful biotechnological means to improve cultivated plants. The advantage of haploids for breeders is that homozygosity can be achieved already in the first generation after dihaploidization, creating doubled haploid plants, without the need of several backcrossing generations required to obtain a high degree of homozygosity. Further, the value of haploids in plant research and breeding lies in the fact that the founder cells of doubled haploids are products of meiosis, so that resultant populations constitute pools of diverse recombinant and at the same time genetically fixed individuals. The generation of doubled haploids thus provides not only perfectly useful genetic variability to select from with regard to crop improvement but is also a valuable means to produce mapping populations, recombinant inbreds as well as instantly homozygous mutants and transgenic lines.

Haploids can be obtained by in vitro or in vivo approaches. However, many species and genotypes are recalcitrant to these processes. Alternatively, substantial changes of the centromere-specific histone H3 variant (CENH3, also called CENP-A), by swapping its N-terminal regions and fusing it to GFP (“GFP-tailswap” CENH3), creates haploid inducer lines in the model plant Arabidopsis thaliana (Ravi and Chan, Nature, 464 (20 10), 615-618; Comai, L, “Genome elimination: translating basic research into a future tool for plant breeding.”, PLoS biology, 12. 6 (2014)). CENH3 proteins are variants of H3 histone proteins that are members of the kinetochore complex of active centromeres. With these “GFP-tailswap” haploid inducer lines, haploidization occurred in the progeny when a haploid inducer plant was crossed with a wild type plant. The haploid inducer line was stable upon selfing, suggesting that a competition between modified and wild type centromere in the developing hybrid embryo results in centromere inactivation of the inducer parent and consequently in uniparental chromosome elimination. As a result, the chromosomes containing the altered CENH3 protein are lost during early embryo development producing haploid progeny containing only the chromosomes of the wild type parent. Thus, haploid plants can be obtained by crossing “GFP-tailswap” plants as haploid inducer to wildtype plants.

WO 2016/030019 and WO 2016/102665 describe an alternative non-transgenic way for modification of the endogenous CENH3 gene(s) in a plant for creation of haploid inducer lines. The authors show that in particular one or more single amino acid substitutions in diverse domains of CENH3 protein result in haploid induction when the mutant plant is crossed with a wildtype plant.

The CENH3 mutants, either as transgenic “tailswap” inducer or as non-transgenic inducer with mutated endogenous CENH3 gene(s), function in Arabidopsis as haploid inducer and can reach rates of up to 10%. However, these data could not be transferred to crop plants. In both maize and rapeseed, the haploid induction rates as such, with up to 3.6% for the transgenic “tailswap” inducer (Kelliher et al. (2016) “Maternal haploids are preferentially induced by CENH3-tailswap transgenic complementation in maize”, Frontiers in plant science, 7, 414.) and up to 2% for the non-transgenic inducer (WO 2016/030019; WO 2016/102665), were much lower than in Arabidopsis and haploid induction was mainly observed on the maternal side.

Another possibility for the induction of haploids in maize is the indeterminate gametophyte (ig) system. A so called mutated ig gene induces haploids of both male (androgenetic) and female (gynogenetic) origin. The ig gene was first described by Kermicle (1969, “Androgenesis conditioned by a mutation in maize”, Science, 166(3911), 1422-1424) as arising spontaneously in the highly inbred Wisconsin-23 (W23) strain. The ig gene is essential for the normal growth and development of the gametophyte and loss of function of the ig gene causes too many or too few nuclei to be produced. In ig lines the developing megagametophyte is released from its normal three mitotic divisions. Lin (1981, Rev. Brasil. Biol. 41(3): 557-63), observed that the presence of mutated ig allows the occurrence of a variable number of mitotic divisions and some of the nuclei degenerate. Following fertilization of the megagametophyte, sperm nuclei occasionally develop androgenetically into paternal haploid embryos. Embryonic development of sperm nuclei in maternal cytoplasm results in the formation of androgenetic haploids. Kermicle et al. (1980, Maize Genet. Coop. Newsl. 54: 84-85), determined that the ig allele is positioned in the long arm of chromosome 3 at 90 cM from the most distal locus in the short arm designated g2 (EP 0 831 689). The presence of the ig allele increases the occurrence of paternal haploids from the natural spontaneous frequency of about 1 per 80,000 to a frequency of 1 to 3% of maize plants observed. This is much lower than the maternal induction rate which is usually around 10%.

It is therefore an objective of the present invention to address one or more of the shortcomings of the prior art.

### SUMMARY OF THE INVENTION

The present inventors have surprisingly found that the combination of a mutated centromere or kinetochore gene, such as CENH3, with a mutated indeterminate gametophyte (ig) gene is particularly suitable in the generation of haploid inducer plants, in particular paternal haploid inducer plants, such as maize (such as Zea mays), sorghum (such as Sorghum bicolor), or rapeseed plants (such as Brassica napus). Haploid induction rates were found to be much higher than resulting from either mutation alone, and even higher than could realistically be expected for such combination.

Accordingly, in an aspect, the present invention relates to a plant or plant part comprising a polynucleic acid encoding a mutated indeterminate gametophyte (ig) protein and a polynucleic acid encoding a mutated centromere or kinetochore protein, wherein said mutated centromere or kinetochore protein preferably is CENH3. The mutated ig and centromere or kinetochore proteins together result in haploid inducing activity, such as in particular paternal haploid inducing activity.

In an aspect, the invention relates to a method for generating a plant or plant part, in particular a haploid plant or plant part, comprising crossing a first plant comprising a polynucleic acid encoding a mutated indeterminate gametophyte (ig) protein and a polynucleic acid encoding a mutated centromere or kinetochore protein, wherein said mutated centromere or kinetochore protein preferably is CENH3, with a second plant and selecting haploid progeny. Optionally, the haploid progeny can be converted into doubled haploid plants or plant parts.

In an aspect, the invention relates to a plant or plant part obtained by or obtainable by a method for generating a plant or plant part, in particular a haploid plant or plant part, comprising crossing a first plant comprising a polynucleic acid encoding a mutated indeterminate gametophyte (ig) protein and a polynucleic acid encoding a mutated centromere or kinetochore protein, wherein said mutated centromere or kinetochore protein preferably is CENH3, with a second plant and selecting haploid progeny. Optionally, the haploid progeny can be converted into doubled haploid plants or plant parts.

In an aspect, the invention relates to the use of a plant or plant part comprising a polynucleic acid encoding a mutated indeterminate gametophyte (ig) protein and a polynucleic acid encoding a mutated centromere or kinetochore protein, wherein said mutated centromere or kinetochore protein preferably is CENH3, as a haploid inducer, preferably a paternal haploid inducer.

In an aspect, the invention relates to Zea mays seed designated igEIN, a representative sample of which has been deposited under NCIMB Accession No. NCIMB 43772, or plants or plant parts grown or obtained therefrom. In an aspect, the invention relates to Zea mays seed as deposited under NCIMB Accession No. NCIMB 43772, or plants or plant parts grown or obtained therefrom.

In an aspect, the invention relates to a method for identifying suitable centromere or kinetochore protein, preferably CENH3, mutants or mutations to be combined with an ig mutant or mutation as described herein elsewhere in order to increase haploid inducing activity or capability, by combining such mutations and analysing resulting haploid inducing activity or capability.

The present inventors have surprisingly found that the plants and methods as described herein have an increased haploid induction rate, in particular paternal haploid induction rate. This allows to increase the efficiency of cytoplasmic male sterility (CMS) conversions based on paternal haploid induction. Further, the provision of paternal haploid inducers is of particular importance. In case many haploids should be produced out of one segregating plant, the use of the maternal system is limited, permitting only one cross, resulting in one to two haploid plants in average. The paternal system gives the possibility to make several crosses using the pollen of the plant to pollinate the paternal inducer. Using a high performing inducer, more haploids can be obtained per single segregating plant. Such system offers opportunities to optimize breeding schemes by more efficient use of genome wide prediction or trait integration. Furthermore, for crops with difficult castration systems, a paternal induction system is preferred. It can be applied using a sterile inducer on the basis of nuclear sterility, which can be pollinated by any fertile line. In addition, after introduction of a haploid selection marker like red roots in maize, the present invention can be used for special cases in new breeding or trait introgression programs for double haploid (DH) production from single segregating plants. Finally, efficient paternal inducers with high induction rate can be used in genome editing, in particular when the paternal inducer simultaneously comprises genome editing machinery.

The present invention is in particular captured by any one or any combination of one or more of the below numbered statements 1 to 125, as such or combined with any other statement and/or embodiments provided herein.
1. A plant or plant part comprising a polynucleic acid encoding a mutated indeterminate gametophyte (ig) protein and a polynucleic acid encoding a mutated centromere or kinetochore protein.
2. The plant or plant part according to statement 1, wherein said polynucleic acid encoding said mutated ig protein comprises an insertion of one or more nucleic acids (compared to the polynucleic acid encoding the wild-type indeterminate gametophyte (ig) protein).
3. The plant or plant part according to any of statements 1 to 2, wherein said polynucleic acid encoding said mutated ig protein comprises a frameshift mutation or a nonsense mutation (compared to the polynucleic acid encoding the wild-type indeterminate gametophyte (ig) protein).
4. The plant or plant part according to any of statements 1 to 3, wherein said polynucleic acid encoding said mutated ig protein comprises a knockout mutation or a knockdown mutation.
5. The plant or plant part according to any of statements 1 to 4, wherein said polynucleic acid encoding said mutated ig protein comprises an insertion of one or more nucleic acids in the ig coding sequence (compared to the polynucleic acid encoding the wild-type indeterminate gametophyte (ig) protein).
6. The plant or plant part according to any of statements 1 to 5, wherein said polynucleic acid encoding said mutated ig protein comprises an insertion of one or more nucleic acids in the LOB domain encoding sequence (compared to the polynucleic acid encoding the wild-type indeterminate gametophyte (ig) protein).
7. The plant or plant part according to any of statements 1 to 6, wherein said polynucleic acid encoding said mutated ig protein comprises an insertion of one or more nucleic acids in the first protein encoding exon, such as ranging from nucleotide positions 431 to 841 of reference Zea mays sequence set forth in SEQ ID NO: 6.
8. The plant or plant part according to any of statements 1 to 7, wherein said polynucleic acid encoding said mutated ig protein comprises an insertion of one or more nucleic acids in the intron preceding the first protein encoding exon.
9. The plant or plant part according to any of statements 1 to 8, wherein said polynucleic acid encoding said mutated ig protein comprises the ig-O allele.
10. The plant or plant part according to any of statements 1 to 9, wherein said polynucleic acid encoding said mutated ig protein comprises the ig-mum allele.
11. The plant or plant part according to any of statements 1 to 10, wherein said polynucleic acid encoding said mutated ig protein comprises an insertion of one or more nucleic acids in an ig codon corresponding to a codon selected from codon 118, 119, or 120 of the wild type Zea mays ig protein, such as set forth SEQ ID NO: 7 or 8, corresponding to a codon selected from codon 191, 192, or 193 of the wild type Sorghum bicolor ig protein, such as set forth in SEQ ID NO: 22, corresponding to a codon selected from codon 143, 144, or 145 of the wild type Sorghum bicolor ig protein, such as set forth in SEQ ID NO: 25, corresponding to a codon selected from codon 94, 95 or 96 of the wild type Brassica napus ig protein, such as set forth in SEQ ID NO: 28 or 31.
12. The plant or plant part according to any of statements 1 to 11, wherein said polynucleic acid encoding said mutated ig protein comprises an insertion of at least 100, preferably at least 200 nucleotides (compared to the polynucleic acid encoding the wild-type indeterminate gametophyte (ig) protein).
13. The plant or plant part according to any of statements 1 to 12, wherein said mutated ig protein comprises an insertion of one or more amino acids and/or substitution of one or more amino acids (compared to the wild-type ig protein).
14. The plant or plant part according to any of statements 1 to 13, wherein said mutated ig protein comprises an insertion of one or more amino acids and/or substitution of one or more amino acids in a region corresponding to amino acid residues 110 to 130 of the wild type Zea mays ig protein, such as set forth in SEQ ID NO: 9 or 10, corresponding to amino acid residues 183 to 203 of the wild type Sorghum bicolor ig protein, such as set forth in SEQ ID NO: 23, corresponding to amino acid residues 135 to 155 of the wild type Sorghum bicolor ig protein, such as set forth in SEQ ID NO: 26, or corresponding to amino acid residues 86 to 106 of the wild type Brassica napus ig protein, such as set forth in SEQ ID NO: 29 or 32.
15. The plant or plant part according to any of statements 1 to 14, wherein said mutated ig protein comprises an insertion of one or more amino acids and/or substitution of one or more amino acids in a region corresponding to amino acid residues 116 to 120, preferably 117 to 119, of the wild type Zea mays ig protein, such as set forth in SEQ ID NO: 9 or 10, corresponding to amino acid residues 189 to 193, preferable 190 to 192, of the wild type Sorghum bicolor ig protein, such as set forth in SEQ ID NO: 23, corresponding to amino acid residues 141 to 145, preferably 142 to 144, of the wild type Sorghum bicolor ig protein, such as set forth in SEQ ID NO: 26, or corresponding to amino acid residues 92 to 96, preferably 93 to 95, of the wild type Brassica napus ig protein, such as set forth in SEQ ID NO: 29 or 32.
16. The plant or plant part according to any of statements 1 to 15, wherein said mutated ig protein is a truncated ig protein.
17. The plant or plant part according to any of statements 1 to 16, wherein said ig is ig1.
18. The plant or plant part according to any of statements 1 to 16, wherein said ig is ig2.
19. The plant or plant part according to any of statements 1 to 18, wherein said plant is from the genus Zea, preferably Zea mays, wherein said wild-type indeterminate gametophyte (ig) protein
a) is encoded by a polynucleic acid comprising the nucleotide sequence of SEQ ID NO: 6 or a sequence which is at least 90% identical, preferably at least 95% identical, more preferable at least 98% identical to SEQ ID NO: 6;
b) is derived from a coding sequence comprising the nucleotide sequence of SEQ ID NO: 7 or 8, or a sequence which is at least 90% identical, preferably at least 95% identical, more preferable at least 98% identical to SEQ ID NO: 7 or 8; or
c) has an amino acid sequence of SEQ ID NO: 9 or 10, or a sequence which is at least 90% identical, preferably at least 95% identical, more preferable at least 98% identical to SEQ ID NO: 9 or 10.
20. The plant or plant part according to any of statements 1 to 18, wherein said plant is from the genus Sorghum, preferably Sorghum bicolor, wherein said wild-type indeterminate gametophyte (ig) protein
a) is encoded by a polynucleic acid comprising the nucleotide sequence of SEQ ID NO: 21 or 24, or a sequence which is at least 90% identical, preferably at least 95% identical, more preferable at least 98% identical to SEQ ID NO: 21 or 24;

b) is derived from a coding sequence comprising the nucleotide sequence of SEQ ID NO: 22 or 25, or a sequence which is at least 90% identical, preferably at least 95% identical, more preferable at least 98% identical to SEQ ID NO: 22 or 25; or
c) has an amino acid sequence of SEQ ID NO: 23 or 26, or a sequence which is at least 90% identical, preferably at least 95% identical, more preferable at least 98% identical to SEQ ID NO: 23 or 26.
21. The plant or plant part according to any of statements 1 to 18, wherein said plant is from the genus Brassica, preferably Brassica napus, wherein said wild-type indeterminate gametophyte (ig) protein
a) is encoded by a polynucleic acid comprising the nucleotide sequence of SEQ ID NO: 27 or 30, or a sequence which is at least 90% identical, preferably at least 95% identical, more preferable at least 98% identical to SEQ ID NO: 27 or 30;
b) is derived from a coding sequence comprising the nucleotide sequence of SEQ ID NO: 28 or 31, or a sequence which is at least 90% identical, preferably at least 95% identical, more preferable at least 98% identical to SEQ ID NO: 28 or 31; or
c) has an amino acid sequence of SEQ ID NO: 29 or 32, or a sequence which is at least 90% identical, preferably at least 95% identical, more preferable at least 98% identical to SEQ ID NO: 29 or 32.
22. The plant or plant part according to any of statements 1 to 18, wherein said plant is from the genus Zea, preferably Zea mays, wherein said mutated indeterminate gametophyte (ig) protein
a) is encoded by a polynucleic acid comprising the nucleotide sequence of SEQ ID NO: 1 or a sequence which is at least 90% identical, preferably at least 95% identical, more preferable at least 98% identical to SEQ ID NO: 1;
b) is derived from a coding sequence comprising the nucleotide sequence of SEQ ID NO: 2 or 3, or a sequence which is at least 90% identical, preferably at least 95% identical, more preferable at least 98% identical to SEQ ID NO: 2 or 3; or
c) has an amino acid sequence of SEQ ID NO: 4 or 5, or which is at least 90% identical, preferably at least 95% identical, more preferable at least 98% identical to SEQ ID NO: 4 or 5.
23. The plant or plant part according to any of statements 1 to 18, wherein said plant is from the genus Sorghum, preferably Sorghum bicolor, wherein said mutated indeterminate gametophyte (ig) protein has an amino acid sequence which is at least 90% identical, preferably at least 95% identical, more preferable at least 98% identical to SEQ ID NO: 23 or 26, and which is not 100% identical respectively to SEQ ID NO: 23 or 26.
24. The plant or plant part according to any of statements 1 to 18, wherein said plant is from the genus Brassica, preferably Brassica napus, wherein said mutated indeterminate gametophyte (ig) protein has an amino acid sequence which is at least 90% identical, preferably at least 95% identical, more preferable at least 98% identical to SEQ ID NO: 29 or 32, and which is not 100% identical respectively to SEQ ID NO: 29 or 32.
25. The plant or plant part according to any of statements 1 to 24, wherein said mutated centromere protein is a mutated histone protein.
26. The plant or plant part according to any of statements 1 to 25, wherein said mutated centromere or kinetochore protein is selected from the group comprising CENH3 or proteins interacting with CENH3.
27. The plant or plant part according to any of statements 1 to 26, wherein said mutated centromere or kinetochore protein is selected from the group comprising CENH3, CENP-C, KNL2, SCM3, SAD2 and SIM3.
28. The plant or plant part according to any of statements 1 to 27, wherein said mutated centromere protein is a mutated CENH3 protein.
29. The plant or plant part according to any of statements 1 to 28, wherein said mutated CENH3 protein comprises one or more mutated amino acids in one or more of the N-terminal domain, the αN-helix, the α1-helix, the loop 1 domain, the α2-helix, the loop 2 domain, the α3-helix, the C-terminal domain of CENH3.
30. The plant or plant part according to any of statements 1 to 29, wherein said mutated CENH3 protein comprises one or more mutated amino acids in one or more of the N-terminal domain corresponding to amino acids 1 to 82 of Arabidopsis thaliana CENH3, the αN-helix corresponding to amino acids 83 to 97 of Arabidopsis thaliana CENH3, the α1-helix to amino acids 103 to 113 of Arabidopsis thaliana CENH3, the loop 1 domain to amino acids 114 to 126 of Arabidopsis thaliana CENH3, the α2-helix to amino acids 127 to 155 of Arabidopsis thaliana CENH3, the loop 2 domain to amino acids 156 to 162 of Arabidopsis thaliana CENH3, the α3-helix to amino acids 163 to 172 of Arabidopsis thaliana CENH3, the C-terminal domain of CENH3 to amino acids 173 to 178 of Arabidopsis thaliana CENH3, preferably wherein said Arabidopsis thaliana CENH3 has an amino acid sequence which is at least 90%, preferably at least 95%, more preferably at least 98% identical to a sequence as set forth in SEQ ID NO: 12.
31. The plant or plant part according to any of statements 1 to 29, wherein said mutated CENH3 protein comprises one or more mutated amino acids in one or more of the N-terminal domain corresponding to amino acids 1 to 62 of Zea mays CENH3, the αN-helix corresponding to amino acids 63 to 77 of Zea mays CENH3, the α1-helix to amino acids 83 to 93 of Zea mays CENH3, the loop 1 domain to amino acids 94 to 106 of Zea mays CENH3, the α2-helix to amino acids 107 to 135 of Zea mays CENH3, the loop 2 domain to amino acids 136 to 142 of Zea mays CENH3, the α3-helix to amino acids 143 to 152 of Zea mays CENH3, the C-terminal domain of CENH3 to amino acids 153 to 157 of Zea mays CENH3, preferably wherein said Zea mays CENH3 has an amino acid sequence which is at least 90%, preferably at least 95%, more preferably at least 98% identical to a sequence as set forth in SEQ ID NO: 14.
32. The plant or plant part according to any of statements 1 to 29, wherein said mutated CENH3 protein comprises one or more mutated amino acids in one or more of the N-terminal domain corresponding to amino acids 1 to 62 of Sorghum bicolor CENH3, the αN-helix corresponding to amino acids 63 to 77 of Sorghum bicolor CENH3, the α1-helix to amino acids 83 to 93 of Sorghum bicolor CENH3, the loop 1 domain to amino acids 94 to 106 of Sorghum bicolor CENH3, the α2-helix to amino acids 107 to 135 of Sorghum bicolor CENH3, the loop 2 domain to amino acids 136 to 142 of Sorghum bicolor CENH3, the α3-helix to amino acids 143 to 152 of Sorghum bicolor CENH3, the C-terminal domain of CENH3 to amino acids 153 to 157 of Sorghum bicolor CENH3, preferably wherein said Sorghum bicolor CENH3 has an amino acid sequence which is at least 90%, preferably at least 95%, more preferably at least 98% identical to a sequence as set forth in SEQ ID NO: 18.
33. The plant or plant part according to any of statements 1 to 29, wherein said mutated CENH3 protein comprises one or more mutated amino acids in one or more of the N-terminal domain corresponding to amino acids 1 to 84 of Brassica napus CENH3, the αN-helix corresponding to amino acids 85 to 99 of Brassica napus CENH3, the α1-helix to amino acids 105 to 115 of Brassica napus CENH3, the loop 1 domain to amino acids 116 to 128 of Brassica napus CENH3, the α2-helix to amino acids 129 to 157 of Brassica napus CENH3, the loop 2 domain to amino acids 158 to 164 of Brassica napus CENH3, the α3-helix to amino acids 165 to 174 of Brassica napus CENH3, the C-terminal domain of CENH3 to amino acids 175 to 180 of Brassica napus CENH3, preferably wherein said Brassica napus CENH3 has an amino acid sequence which is at least 90%, preferably at least 95%, more preferably at least 98% identical to a sequence as set forth in SEQ ID NO: 16.
34. The plant or plant part according to any of statements 1 to 29, wherein said mutated CENH3 protein comprises one or more mutated amino acids in the N-terminal domain of CENH3.
35. The plant or plant part according to statement 34, wherein said N-terminal domain of CENH3 corresponds to amino acids 1 to 82 of reference Arabidopsis thaliana CENH3 protein, preferably wherein said Arabidopsis thaliana CENH3 protein has an amino acid sequence which is at least 90%, preferably at least 95%, more preferably at least 98% identical to a sequence as set forth in SEQ ID NO: 12.
36. The plant or plant part according to any of statements 1 to 29, wherein said mutated CENH3 protein comprises one or more mutated amino acids corresponding to positions 3, 17, 32, 35, 9, 24, 29, 40, 42, 50, 55, 57, 61, 74 or 82 of reference Arabidopsis thaliana CENH3 protein, preferably wherein said Arabidopsis thaliana CENH3 protein has an amino acid sequence which is at least 90%, preferably at least 95%, more preferably at least 98% identical to a sequence as set forth in SEQ ID NO: 12.
37. The plant or plant part according to any of statements 1 to 29, wherein said mutated CENH3 protein comprises one or more mutated amino acids corresponding to positions 3, 17, 32 or 35 of Arabidopsis thaliana CENH3 protein if said plant or plant part is from the genus Zea, preferably Zea mays, preferably wherein said Arabidopsis thaliana CENH3 protein has an amino acid sequence which is at least 90%, preferably at least 95%, more preferably at least 98% identical to a sequence as set forth in SEQ ID NO: 12.
38. The plant or plant part according to any of statements 1 to 37, wherein said mutated CENH3 protein comprises one or more mutated amino acids at positions 3, 16, 32 or 35 of CENH3 protein of a plant or plant part from the genus Zea, preferably Zea mays, preferably wherein said Zea mays CENH3 protein has an amino acid sequence which is at least 90%, preferably at least 95%, more preferably at least 98% identical to a sequence as set forth in SEQ ID NO: 14.
39. The plant or plant part according to any of statements 1 to 29, wherein said mutated CENH3 protein comprises one or more mutated amino acids corresponding to positions 9, 24, 29, 32, 40, 42, 50, 55, 57 or 61 of reference Arabidopsis thaliana CENH3 protein if said plant or plant part is from the genus Brassica, preferably Brassica napus, preferably wherein said Arabidopsis thaliana CENH3 protein has an amino acid sequence which is at least 90%, preferably at least 95%, more preferably at least 98% identical to a sequence as set forth in SEQ ID NO: 12.
40. The plant or plant part according to any of statements 1 to 29, wherein said mutated CENH3 protein comprises one or more mutated amino acids at positions 9, 24, 29, 30, 33, 41, 43, 50, 55, 57 or 61 of CENH3 protein of a plant or plant part from the genus Brassica, preferably Brassica napus, preferably wherein said Brassica napus CENH3 protein has an amino acid sequence which is at least 90%, preferably at least 95%, more preferably at least 98% identical to a sequence as set forth in SEQ ID NO: 16.
41. The plant or plant part according to any of statements 1 to 29, wherein said mutated CENH3 protein comprises one or more mutated amino acids corresponding to positions 42 or 74 of reference Arabidopsis thaliana CENH3 protein if said plant or plant part is from the genus Sorghum, preferably Sorghum bicolor, preferably wherein said Arabidopsis thaliana CENH3 protein has an amino acid sequence which is at least 90%, preferably at least 95%, more preferably at least 98% identical to a sequence as set forth in SEQ ID NO: 12.
42. The plant or plant part according to any of statements 1 to 29, wherein said mutated CENH3 protein comprises one or more mutated amino acids at positions 42 or 55 of CENH3 protein of a plant or plant part from the genus Sorghum, preferably Sorghum bicolor, preferably wherein said Sorghum bicolor CENH3 protein has an amino acid sequence which is at least 90%, preferably at least 95%, more preferably at least 98% identical to a sequence as set forth in SEQ ID NO: 18.
43. The plant or plant part according to any of statements 1 to 29, wherein said mutated CENH3 protein comprises one or more mutated amino acids corresponding to positions 104, 109, 120, 148, 175, 130, 151, 157, 158, 164, 166, 83, 86, 124, 127, 132, 136, 152, 155 or 172 of reference Arabidopsis thaliana CENH3 protein, preferably wherein said Arabidopsis thaliana CENH3 protein has an amino acid sequence which is at least 90%, preferably at least 95%, more preferably at least 98% identical to a sequence as set forth in SEQ ID NO: 12.
44. The plant or plant part according to any of statements 1 to 29, wherein said mutated CENH3 protein comprises one or more mutated amino acids corresponding to positions 104, 109, 120, 148 or 175 of reference Arabidopsis thaliana CENH3 protein if said plant or plant part is from the genus Zea, preferably Zea mays, preferably wherein said Arabidopsis thaliana CENH3 protein has an amino acid sequence which is at least 90%, preferably at least 95%, more preferably at least 98% identical to a sequence as set forth in SEQ ID NO: 12.
45. The plant or plant part according to any of statements 1 to 29, wherein said mutated CENH3 protein comprises one or more mutated amino acids at positions 84, 89, 100, 128 or 155 of CENH3 protein of a plant or plant part from the genus Zea, preferably Zea mays, preferably wherein said Zea mays CENH3 protein has an amino acid sequence which is at least 90%, preferably at least 95%, more preferably at least 98% identical to a sequence as set forth in SEQ ID NO: 14.
46. The plant or plant part according to any of statements 1 to 29, wherein said mutated CENH3 protein comprises one or more mutated amino acids corresponding to positions 130 of reference Arabidopsis thaliana CENH3 protein if said plant or plant part is from the genus Sorghum, preferably Sorghum bicolor protein, preferably wherein said Arabidopsis thaliana CENH3 has an amino acid sequence which is at least 90%, preferably at least 95%, more preferably at least 98% identical to a sequence as set forth in SEQ ID NO: 12.
47. The plant or plant part according to any of statements 1 to 29, wherein said mutated CENH3 protein comprises one or more mutated amino acids at positions 110 or 157 of CENH3 protein of a plant or plant part from the genus Sorghum, preferably Sorghum bicolor, preferably wherein said Sorghum bicolor CENH3 protein has an amino acid sequence which is at least 90%, preferably at least 95%, more preferably at least 98% identical to a sequence as set forth in SEQ ID NO: 18.
48. The plant or plant part according to any of statements 1 to 29, wherein said mutated CENH3 protein comprises one or more mutated amino acids corresponding to positions 130, 151, 157, 158, 164 or 166 of reference Arabidopsis thaliana CENH3 protein if said plant or plant part is from the genus Brassica, preferably Brassica napus, preferably wherein said Arabidopsis thaliana CENH3 protein has an amino acid sequence which is at least 90%, preferably at least 95%, more preferably at least 98% identical to a sequence as set forth in SEQ ID NO: 12.
49. The plant or plant part according to any of statements 1 to 29, wherein said mutated CENH3 protein comprises one or more mutated amino acids corresponding to positions 132, 153, 159, 160, 166 or 168 of CENH3 protein of a plant or plant part from the genus Brassica, preferably Brassica napus, preferably wherein said Brassica napus CENH3 protein has an amino acid sequence which is at least 90%, preferably at least 95%, more preferably at least 98% identical to a sequence as set forth in SEQ ID NO: 16.
50. The plant or plant part according to any of statements 25 to 49, wherein said mutated protein comprises one or more amino acid substitution or wherein said one or more mutated amino acid is one or more amino acid substitution.
51. The plant or plant part according to any of statements 25 to 49, comprising one to seven mutations, such as one to seven amino acid substitutions.
52. The plant or plant part according to any of statements 25 to 49, comprising one mutation, such as one amino acid substitution.
53. The plant or plant part according to any of statements 1 to 29, wherein said plant is Zea mays and wherein said mutated centromere or kinetochore protein is mutated CENH3 protein having an amino acid substitution corresponding to position 35 of Zea mays CENH3, preferably an amino acid substitution corresponding to position 35 of SEQ ID NO: 14 or at position 35 of SEQ ID NO: 14, preferably wherein said amino acid substitution is 35K, such as E35K.
54. The plant or plant part according to any of statements 1 to 53, wherein said polynucleic acid encoding a mutated indeterminate gametophyte (ig) protein and said polynucleic acid encoding a mutated centromere or kinetochore protein are operatively linked to one or more regulatory sequences.
55. The plant or plant part according to any of statements 1 to 54, wherein said mutated indeterminate gametophyte (ig) protein and said mutated centromere or kinetochore protein are capable of being expressed in said plant or plant part.
56. The plant or plant part according to any of statements 1 to 55, wherein said mutated indeterminate gametophyte (ig) protein confers haploid inducer activity or is an enhancer for haploid induction capability.
57. The plant or plant part according to any of statements 1 to 56, wherein said mutated centromere or kinetochore protein confers haploid inducer activity or is an enhancer for haploid induction capability.
58. The plant or plant part according to any of statements 1 to 57, wherein said polynucleic acid encoding a mutated indeterminate gametophyte (ig) protein encodes a mutated endogenous indeterminate gametophyte (ig) protein.
59. The plant or plant part according to any of statements 1 to 58, wherein said polynucleic acid encoding a mutated indeterminate gametophyte (ig) protein encodes a mutated endogenous indeterminate gametophyte (ig) protein in its native genomic locus.
60. The plant or plant part according to any of statements 1 to 59, wherein said polynucleic acid encoding a mutated centromere or kinetochore protein encodes a mutated endogenous centromere or kinetochore protein.
61. The plant or plant part according to any of statements 1 to 60, wherein said polynucleic acid encoding a mutated centromere or kinetochore protein encodes a mutated endogenous centromere or kinetochore protein in its native genomic locus.
62. The plant or plant part according to any of statements 1 to 61, wherein said polynucleic acid encoding a mutated indeterminate gametophyte (ig) protein and/or said polynucleic acid encoding a mutated centromere or kinetochore protein is homozygous.
63. The plant or plant part according to any of statements 1 to 62, wherein said polynucleic acid encoding a mutated indeterminate gametophyte (ig) protein and/or said polynucleic acid encoding a mutated centromere or kinetochore protein is heterozygous.
64. The plant or plant part according to any of statements 1 to 63, wherein said plant or plant part is a crop plant or plant part.
65. The plant or plant part according to any of statements 1 to 64, wherein said plant or plant part is selected from the group comprising the genera Zea, Sorghum, and Brassica.
66. The plant or plant part according to statement 65, wherein said plant or plant part is selected from the group comprising the genera Zea and Sorghum.
67. The plant or plant part according to statement 66, wherein said plant or plant part is from the genus Zea.
68. The plant or plant part according to statement 65, wherein said plant or plant part is selected from the group comprising the species Zea mays, Sorghum bicolor, and Brassica napus.
69. The plant or plant part according to statement 66, wherein said plant or plant part is selected from the group comprising the species Zea mays and Sorghum bicolor.
70. The plant or plant part according to statement 67, wherein said plant or plant part is from the species Zea mays.
71. The plant or plant part according to any of statements 1 to 70, wherein said plant part is a plant cell, tissue, organ, or seed.
72. The plant or plant part according to any of statements 1 to 71, wherein said plant or plant part is diploid.
73. The plant or plant part according to any of statements 1 to 71, wherein said plant or plant part is haploid.
74. The plant or plant part according to any of statements 1 to 71, wherein said plant or plant part is dihaploid.
75. The plant or plant part according to any of statements 1 to 71, wherein said plant or plant part is trihaploid.
76. The plant or plant part according to any of statements 1 to 71, wherein said plant or plant part is doubled haploid.
77. The plant or plant part according to any of statements 1 to 71, wherein said plant or plant part is doubled dihaploid.
78. The plant or plant part according to any of statements 1 to 71, wherein said plant or plant part is doubled trihaploid.
79. The plant according to any of statements 1 to 78, further comprising a polynucleic acid encoding a site-directed DNA or RNA binding protein.
80. The plant according to any of statements 1 to 79, further comprising a polynucleic acid encoding a site-directed (mutated) DNA or RNA nuclease.
81. The plant according to statement 80, wherein said site-directed (mutated) nuclease is selected from the group comprising meganucleases (MNs), zinc-finger nucleases (ZFNs), transcription-activator like effector nucleases (TALENs), (mutated) Cas nucleases/effector proteins, such as Cas9 nuclease, Cfp1 nuclease, MAD7 nuclease, dCas9-FokI, dCpf1-FokI, dMAD7 nuclease-FokI, chimeric Cas9-cytidine deaminase, chimeric Cas9-adenine deaminase, chimeric FENI-FokI, and Mega-TALs, a nickase Cas9 (nCas9), chimeric dCas9 non-FokI nuclease, dCpf1 non-FokI nuclease and dMAD7 non-FokI nuclease.
82. The plant according to any of statements 80 to 81, wherein if said site-directed (mutated) nuclease is a (mutated) Cas effector protein, then said plant further comprises a polynucleic acid encoding a gRNA and optionally a polynucleic acid encoding a tracrRNA.
83. A plant or plant part obtainable by crossing a first plant which is a plant according to any of statements 1 to 82 with a second plant.
84. A method for generating a plant or plant part, comprising providing a haploid, dihaploid, or trihaploid plant resulting from crossing a first plant which is a plant according to any of statements 1 to 72 or 79 to 82 with a second plant and converting the haploid, dihaploid, or trihaploid plant or plant part into a doubled haploid, doubled dihaploid, or doubled trihaploid plant or plant part.
85. A method for generating a plant or plant part, comprising crossing a first plant which is a plant according to any of statements 1 to 72 or 76 to 82 with a second plant.
86. A method for generating a haploid, dihaploid, or trihaploid plant, comprising crossing a first plant or plant part which is a plant according to any of statements 1 to 72 or 76 to 82 with a second plant and selecting a haploid, dihaploid, or trihaploid offspring plant or plant part.
87. A method for generating a doubled haploid, doubled dihaploid, or doubled trihaploid plant, comprising crossing a first plant or plant part which is a plant according to any of statements 1 to 72 or 76 to 82 with a second plant, selecting a haploid, dihaploid, or trihaploid offspring plant or plant part, and converting the haploid, dihaploid, or trihaploid plant or plant part into a doubled haploid, doubled dihaploid, or doubled trihaploid plant or plant part.
88. A method of modifying plant genomic DNA, comprising: a) providing a first plant which is a plant according to any of statements 76 to 82; b) providing a second plant (comprising the plant genomic DNA which is to be modified); c) pollinating the second maize plant with pollen from the first plant; and d) selecting at least one haploid, dihaploid or trihaploid progeny produced by the pollination of step (c) (wherein the haploid, dihaploid or trihaploid progeny comprises the genome of the second plant but not the first plant, and the genome of the haploid, dihaploid or trihaploid progeny has been modified by the site-directed DNA or RNA binding protein delivered by the first plant).
89. The method of statement 88, wherein the modified haploid progeny is treated with a chromosome doubling agent, thereby creating a modified doubled haploid progeny.
90. The method of statement 89, wherein the chromosome doubling agent is colchicine, pronamide, dithipyr, trifluralin, or another known anti-microtubule agent.
91. The method according to any of statements 84 to 90, wherein said second plant is from the same species as said first plant.
92. The method according to any of statements 84 to 91, wherein said second plant has a different haplotype as said first plant.
93. The method according to any of statements 84 to 92, wherein said second plant is diploid, tetraploid, or hexaploid.
94. The method according to any of statements 84 to 93, wherein said second plant does not comprise a polynucleic acid encoding a mutated indeterminate gametophyte (ig) protein and/or a polynucleic acid encoding a mutated centromere or kinetochore protein.
95. The method according to any of statements 84 to 94, wherein said second plant is not a haploid inducer.
96. A plant or plant part obtainable by the method according to any of statements 84 to 95.
97. Use of a plant or plant part according to any of statements 1 to 83 or 96 as a haploid inducer.
98. Use of a plant or plant part according to any of statements 1 to 83 or 96 as a paternal haploid inducer.
99. The plant or plant part according to statement 71, wherein said plant part is pollen.
100. The plant or plant part according to any of statements 1 to 82 which is not exclusively obtained by means of an essentially biological process.
101. A method for identifying a plant or plant part, comprising detecting (in a sample from a plant or plant part, such as a sample comprising (genomic) DNA from a plant or plant part) a mutated indeterminate gametophyte protein and a mutated centromere or kinetochore protein or detecting a polynucleic acid encoding an indeterminate gametophyte protein comprising a mutation and a polynucleic acid encoding a centromere or kinetochore protein comprising a mutation.
102. The method according to statement 101, comprising detecting a mutated indeterminate gametophyte protein and a mutated centromere or kinetochore protein or detecting a polynucleic acid encoding an indeterminate gametophyte protein comprising a mutation and a polynucleic acid encoding a centromere or kinetochore protein comprising a mutation as defined in any of statements 1 to 63.
103. The method according to any of statements 101 to 102, wherein said plant or plant part is a plant or plant part according to any of statements 1 to 83, 96, or 100.
104. The method according to any of statements 101 to 103, which is a method for detecting a plant or plant part having haploid inducer activity or enhanced haploid inducer activity.
105. The method according to any of statements 101 to 104, which is a method for detecting a plant or plant part having paternal haploid inducer activity or enhanced paternal haploid inducer activity.
106. The method according to any of statements 101 to 105, comprising marker-assisted selection.
107. The method according to any of statements 101 to 106, comprising detecting a (molecular or genetic) marker associated with or linked with said polynucleic acid encoding an indeterminate gametophyte protein comprising a mutation and detecting a (molecular or genetic) marker associated with or linked with a polynucleic acid encoding a centromere or kinetochore protein comprising a mutation.
108. The method according to statement 107, wherein said (molecular or genetic) marker comprises or encodes a polynucleic acid comprising said mutation, the complement thereof, or the reverse complement thereof.
109. The method according to any of statements 107 to 108, wherein said (molecular or genetic) marker comprises a primer or a probe.
110. The method according to any of statements 101 to 109, wherein said detecting comprises sequencing, hybridization based methods (such as (dynamic) allele-specific hybridization, molecular beacons, SNP microarrays), enzyme based methods (such as PCR, KASP (Kompetitive Allele Specific PCR), RFLP, ALFP, RAPD, Flap endonuclease, primer extension, 5′-nuclease, oligonucleotide ligation assay), post-amplification methods based on physical properties of DNA (such as single strand conformation polymorphism, temperature gradient gel electrophoresis, denaturing high performance liquid chromatography, high-resolution melting of the entire amplicon, use of DNA mismatch-binding proteins, SNPlex, surveyor nuclease assay).
111. A method for generating a plant or plant part, comprising the steps of:
A) providing a plant or plant part; and
   (ii) mutating one or more (endogenous) ig allele, gene, or protein encoding polynucleic acid, and mutating one or more (endogenous) centromere or kinetochore protein allele, gene, or protein encoding polynucleic acid and/or (genomically) introducing one or more mutated ig allele, gene, or protein encoding polynucleic acid, and one or more mutated centromere or kinetochore protein allele, gene, or protein encoding polynucleic acid; or
B) providing a plant or plant part comprising one or more (endogenous) mutated ig allele, gene, or protein encoding polynucleic acid, and/or (genomically) one or more (genomically) introduced mutated ig allele, gene, or protein encoding polynucleic acid; and
   (ii) mutating one or more (endogenous) centromere or kinetochore protein allele, gene, or protein encoding polynucleic acid and/or (genomically) introducing one or more mutated centromere or kinetochore protein allele, gene, or protein encoding polynucleic acid; or
C) (i) providing a plant or plant part comprising one or more (endogenous) mutated centromere or kinetochore protein allele, gene, or protein encoding polynucleic acid, and/or one or more (genomically) introduced mutated centromere or kinetochore allele, gene, or protein encoding polynucleic acid; and
   (ii) Mutating one or more (endogenous) ig allele, gene, or protein encoding polynucleic acid and/or (genomically) introducing one or more mutated ig allele, gene, or protein encoding polynucleic acid.
112. The method for generating a plant or plant part according to statement 111, wherein said plant or plant part is a plant or plant part according to any of statements 1 to 82.
113. The method for generating a plant or plant part according to any of statements 11 to 112, wherein said mutation(s) is (are) as defined in any of statements 1 to 63.
114. A method for generating a plant or plant part, preferably a plant or plant part according to any of statements 1-82, comprising the steps of:
   a) mutagenizing a plant or part thereof and identifying a plant comprising a polynucleic acid encoding a mutated indeterminate gametophyte (ig) protein, preferably as defined in any of statements 2-24, 54, 55, 56, 58, 59, 62, or 63; and
   b) mutagenizing a plant identified in step a) or part thereof or a progeny thereof comprising the polynucleic acid encoding a mutated indeterminate gametophyte (ig) protein, and identifying a plant comprising further a polynucleic acid encoding a mutated centromere or kinetochore protein, preferably as defined in any of statements 25-53, 54, 55, 57, 60, 61, 62, or 63;
or
   A) mutagenizing a plant or part thereof and identifying a plant comprising a polynucleic acid encoding a mutated centromere or kinetochore protein as defined in any of statements 25-53, 54, 55, 57, 60, 61, 62, or 63; and
   B) mutagenizing a plant identified in step a) or part thereof or a progeny thereof comprising the polynucleic acid encoding a mutated centromere or kinetochore protein, and identifying a plant comprising further a polynucleic acid encoding a mutated indeterminate gametophyte (ig) protein as defined in any of statements 2-24, 54, 55, 56, 58, 59, 62, or 63;
or
   mutagenizing a plant or part thereof and identifying a plant or plant part comprising a polynucleic acid encoding a mutated ig protein and a polynucleic acid encoding a mutated centromere or kinetochore protein, preferably a plant or plant part according to any of the statements 1-82.
115. The method according to any of statements 111 to 114, wherein said mutating or mutagenizing comprises random mutagenesis or site-directed mutagenesis.
116. The method according to any of statements 111 to 115, wherein said mutating or mutagenizing comprises irradiation, such as UV, X-ray, or gamma ray radiation, or chemical mutagenesis, such as ethyl methanesulfonate (EMS), ethylnitrosourea (ENU), or dimethylsulfate (DMS).
117. The method according to any of statements 111 to 116, wherein said mutating or mutagenizing comprises TILLING.
118. The method according to any of statements 111 to 115, wherein said mutating or mutagenizing comprises the use of a site-directed (mutated) DNA or RNA nuclease.
119. The method according to statement 118, wherein said site-directed (mutated) DNA or RNA nuclease is selected from the group comprising meganucleases (MNs), zinc-finger nucleases (ZFNs), transcription-activator like effector nucleases (TALENs), (mutated) Cas nucleases/effector proteins, such as Cas9 nuclease, Cfp1 nuclease, MAD7 nuclease, dCas9-FokI, dCpf1-FokI, dMAD7 nuclease-FokI, chimeric Cas9-cytidine deaminase, chimeric Cas9-adenine deaminase, chimeric FENI-FokI, and Mega-TALs, a nickase Cas9 (nCas9), chimeric dCas9 non-FokI nuclease, dCpf1 non-FokI nuclease and dMAD7 non-FokI nuclease.
120. The method according to any of statements 111 to 115, wherein said mutating or mutagenizing comprises the use of a CRISPR/Cas system.
121. The method according to statement 120, wherein said CRISPR/Cas system comprises a guide RNA and a Cas effector protein, and optionally a tracrRNA.
122. The method according to statement 121, wherein said Cas effector protein is Cas9 or Cas12 (Cpf1).
123 The method according to any of statements 121 or 122, wherein said Cas effector protein is a nickase or a catalytically inactive Cas effective protein.
124. The method according to any of statements 121 to 123, wherein said Cas effector protein is fused to a heterologous protein (domain), preferably a heterologous protein domain having enzymatic activity.
125. The method according to any of statements 121 to 124, wherein said Cas effector protein is fused to an adenine deaminase or a cytidine deaminase (domain).
126. A Zea mays seed as deposited under NCIMB Deposit number NCIMB 43772.
127. A (igEIN) Zea Mays seed, a representative sample of which has been deposited under NCIMB Deposit No. NCIMB 43772.
128. A Zea mays plant grown or obtained from the seed according to statement 126 or 127.
129. A Zea mays plant part grown or obtained from the seed according to statement 126 or 127 or obtained from the plant according to statement 128.
130. A method for identifying or selecting a plant or plant part, such as a plant or plant part having (enhanced) haploid inducing activity or capability, comprising:
i) providing a plant or plant part having reduced expression, stability, and/or activity of an indeterminate gametophyte (ig) gene, mRNA, or protein;
ii) mutating a gene encoding a centromere or kinetochore protein, preferably CENH3; and
iii) analysing haploid inducing activity or capability in said plant or plant part, or offspring thereof;
optionally further comprising:
iv) selecting a plant or plant part having (enhanced) haploid inducing activity or capability.
131. A method for identifying or selecting a plant or plant part, such as a plant or plant part having (enhanced) haploid inducing activity or capability, comprising:
i) providing a first plant having reduced expression, stability, and/or activity of an indeterminate gametophyte (ig) gene, mRNA, or protein;
ii) crossing said first plant with a second plant having a gene encoding a mutated centromere or kinetochore protein, preferably CENH3; and
iii) analysing haploid inducing activity or capability in the resulting offspring thereof; optionally further comprising:
iv) selecting a plant or plant part having (enhanced) haploid inducing activity or capability.
132. Use of a plant or plant part having reduced expression, stability, and/or activity of an indeterminate gametophyte (ig) gene, mRNA, or protein for screening for or identifying centromere or kinetochore protein, preferably CENH3, mutations conferring or enhancing haploid inducing activity or capability.

### BRIEF DESCRIPTION OF THE FIGURES

FIG. 1: Protein alignment of various CENH3 orthologues. The amino acid sequences represented are the wildtype CENH3 protein sequences, which for Arabidopsis thaliana is provided in SEQ ID NO: 12, for Beta vulgaris is provided in SEQ ID NO: 34, for Brassica napus is provided in SEQ ID NO: 16, for Zea mays is provided in SEQ ID NO: 14 and for Sorghum bicolor is provided in SEQ ID NO: 18.

### DETAILED DESCRIPTION OF THE INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6, or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

Standard reference works setting forth the general principles of recombinant DNA technology include Molecular Cloning: A Laboratory Manual, 4th ed., (Green and Sambrook et al., 2012, Cold Spring Harbor Laboratory Press); Current Protocols in Molecular Biology, ed. Ausubel et al., Greene Publishing and Wiley-Interscience, New York, 1992 (with periodic updates) (“Ausubel et al. 1992”); the series Methods in Enzymology (Academic Press, Inc.); Innis et al., PCR Protocols: A Guide to Methods and Applications, Academic Press: San Diego, 1990; PCR 2: A Practical Approach (M. J. MacPherson, B. D. Hames and G. R. Taylor eds. (1995); Harlow and Lane, eds. (1988) Antibodies, a Laboratory Manual; and Animal Cell Culture (R. I. Freshney, ed. (1987). General principles of microbiology are set forth, for example, in Davis, B. D. et al., Microbiology, 3rd edition, Harper & Row, publishers, Philadelphia, Pa. (1980).

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilised and structural or logical changes may be made without departing from the scope of the present invention. The following detailed description, therefore, is not to be taken in a limiting sense, and the scope of the present invention is defined by the appended claims. Preferred statements (features) and embodiments of this invention are set herein below. Each statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous.

In an aspect, the invention relates to a plant or plant part comprising or expressing a polynucleic acid encoding a mutated indeterminate gametophyte (ig) protein and a polynucleic acid encoding a mutated centromere or kinetochore protein, preferably mutated CENH3.

In an aspect, the invention relates to a plant or plant part comprising or expressing a mutated indeterminate gametophyte (ig) allele and a mutated centromere or kinetochore protein allele, preferably mutated CENH3.

In an aspect, the invention relates to a plant or plant part comprising or expressing a mutated indeterminate gametophyte (ig) gene and a mutated centromere or kinetochore gene, preferably mutated CENH3.

In an aspect, the invention relates to a plant or plant part comprising or expressing a mutated indeterminate gametophyte (ig) protein and a mutated centromere or kinetochore protein, preferably mutated CENH3.

In an aspect, the invention relates to a plant or plant part comprising or expressing a polynucleic acid encoding an indeterminate gametophyte (ig) protein conferring or enhancing haploid inducing activity or capability and a polynucleic acid encoding a centromere or kinetochore protein, preferably CENH3, conferring or enhancing haploid inducing activity or capability.

In an aspect, the invention relates to a plant or plant part comprising or expressing an indeterminate gametophyte (ig) allele conferring or enhancing haploid inducing activity or capability and a centromere or kinetochore protein allele, preferably CENH3, conferring or enhancing haploid inducing activity or capability.

In an aspect, the invention relates to a plant or plant part comprising or expressing an indeterminate gametophyte (ig) gene conferring or enhancing haploid inducing activity or capability and a centromere or kinetochore, preferably CENH3, gene conferring or enhancing haploid inducing activity or capability.

In an aspect, the invention relates to a plant or plant part comprising or expressing an indeterminate gametophyte (ig) protein conferring or enhancing haploid inducing activity or capability and a centromere or kinetochore protein, preferably CENH3, conferring or enhancing haploid inducing activity or capability.

In an aspect, the invention relates to a plant or plant part having reduced expression, stability, and/or activity of an indeterminate gametophyte (ig) gene, mRNA, or protein and comprising a polynucleic acid encoding a mutated centromere or kinetochore protein, preferably mutated CENH3.

In an aspect, the invention relates to a plant or plant part having reduced expression, stability, and/or activity of an indeterminate gametophyte (ig) gene, mRNA, or protein and comprising a mutated centromere or kinetochore protein allele, preferably mutated CENH3.

In an aspect, the invention relates to a plant or plant part having reduced expression, stability, and/or activity of an indeterminate gametophyte (ig) gene, mRNA, or protein and comprising a mutated centromere or kinetochore gene, preferably mutated CENH3.

In an aspect, the invention relates to a plant or plant part having reduced expression, stability, and/or activity of an indeterminate gametophyte (ig) gene, mRNA, or protein and comprising a mutated centromere or kinetochore protein, preferably mutated CENH3.

In an aspect, the invention relates to a plant or plant part having reduced expression, stability, and/or activity of an indeterminate gametophyte (ig) gene, mRNA, or protein and comprising a polynucleic acid encoding a centromere or kinetochore protein, preferably CENH3, conferring or enhancing haploid inducing activity or capability.

In an aspect, the invention relates to a plant or plant part having reduced expression, stability, and/or activity of an indeterminate gametophyte (ig) gene, mRNA, or protein and comprising a centromere or kinetochore protein allele, preferably CENH3, conferring or enhancing haploid inducing activity or capability.

In an aspect, the invention relates to a plant or plant part having reduced expression, stability, and/or activity of an indeterminate gametophyte (ig) gene, mRNA, or protein and comprising a centromere or kinetochore gene, preferably CENH3, conferring or enhancing haploid inducing activity or capability.

In an aspect, the invention relates to a plant or plant part having reduced expression, stability, and/or activity of an indeterminate gametophyte (ig) gene, mRNA, or protein and comprising a centromere or kinetochore protein, preferably CENH3, conferring or enhancing haploid inducing activity or capability.

In an aspect, the invention relates to a method for identifying or selecting a plant or plant part, such as a plant or plant part having (enhanced) haploid inducing activity or capability, comprising:
i) providing a plant or plant part having reduced expression, stability, and/or activity of an indeterminate gametophyte (ig) gene, mRNA, or protein such as an ig gene according to the invention as described herein;
ii) mutating a gene encoding a centromere or kinetochore protein, preferably CENH3; and
iii) analysing haploid inducing activity or capability in said plant or plant part, or offspring thereof;
optionally further comprising:
iv) selecting a plant or plant part having (enhanced) haploid inducing activity or capability.

Such method allows for the identification of suitable centromere or kinetochore protein, preferably CENH3, mutations to be combined with mutated ig for generating haploid inducers or for enhancing haploid induction. Mutagenesis of a centromere or kinetochore protein can be performed as described herein elsewhere, including but not limited to random mutagenesis, such as TILLING, or site directed mutagenesis, such as genome editing (e.g. CRISPR/Cas mediated).

In an aspect, the invention relates to a method for identifying or selecting a plant or plant part, such as a plant or plant part having (enhanced) haploid inducing activity or capability, comprising:
i) providing a plant having reduced expression, stability, and/or activity of an indeterminate gametophyte (ig) gene, mRNA, or protein such as an ig gene according to the invention as described herein;
ii) crossing said plant with a plant having a gene encoding a mutated centromere or kinetochore protein, preferably CENH3; and
iii) analysing haploid inducing activity or capability in the resulting offspring thereof;
optionally further comprising:
iv) selecting a plant or plant part having (enhanced) haploid inducing activity or capability.

Such method allows for the identification of suitable centromere or kinetochore protein, preferably CENH3, mutations to be combined with mutated ig for generating haploid inducers or for enhancing haploid induction.

In a related aspect, the invention relates to the use of a plant or plant part having reduced expression, stability, and/or activity of an indeterminate gametophyte (ig) gene, mRNA, or protein, such as an ig gene according to the invention as described herein, for screening for or identifying centromere or kinetochore protein, preferably CENH3, mutations conferring or enhancing haploid inducing activity or capability.

The skilled person will understand that the analysis of (enhanced) haploid inducing activity or capability may encompass determining the amount or fraction of haploid inducers, such as haploid inducers resulting from a population of seeds or other plant parts such as propagative plant parts. Enhanced haploid inducing activity or capability can be identified by a (relative) increase in amount of haploid inducer (offspring).

The term “plant” according to the present invention includes whole plants or parts of such a whole plant. Whole plants preferably are seed plants, or a crop. “Parts of a plant” are e.g. shoot vegetative organs/structures, e.g., leaves, stems and tubers; roots, flowers and floral organs/structures, e.g. bracts, sepals, petals, stamens, carpels, anthers and ovules; pollen, seed, including embryo, endosperm, and seed coat; fruit and the mature ovary; plant tissue, e.g. vascular tissue, ground tissue, and the like; and cells, e.g. guard cells, egg cells, pollen, trichomes and the like; and progeny of the same. Parts of plants may be attached to or separate from a whole intact plant. Such parts of a plant include, but are not limited to, organs, tissues, and cells of a plant, and preferably pollen (or seeds). A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant. “Plant cell culture” means cultures of plant units such as, for example, protoplasts, cell culture cells, cells in plant tissues, pollen, pollen tubes, ovules, embryo sacs, zygotes and embryos at various stages of development. “Plant material” refers to leaves, stems, roots, flowers or flower parts, fruits, pollen, egg cells, zygotes, pollen, seeds, cuttings, cell or tissue cultures, or any other part or product of a plant. This also includes callus or callus tissue as well as extracts (such as extracts from taproots) or samples. A “plant organ” is a distinct and visibly structured and differentiated part of a plant such as a root, stem, leaf, flower bud, or embryo. “Plant tissue” as used herein means a group of plant cells organized into a structural and functional unit. Any tissue of a plant in planta or in culture is included. This term includes, but is not limited to, whole plants, plant organs, plant pollen, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue. In certain embodiments, the plant part or derivative is not (functional) propagation material, such as germplasm, a seed, or plant embryo or other material from which a plant can be regenerated. In certain embodiments, the plant part or derivative does not comprise (functional) male and female reproductive organs. In certain embodiments, the plant part or derivative is or comprises propagation material, but propagation material which does not or cannot be used (anymore) to produce or generate new plants, such as propagation material which have been chemically, mechanically or otherwise rendered non-functional, for instance by heat treatment, acid treatment, compaction, crushing, chopping, etc. In certain embodiments, the plant part or derivative is (functional) propagation material, such as germplasm, a seed, or plant embryo or other material from which a plant can be regenerated. In certain embodiments, the plant part or derivative comprises (functional) male and female reproductive organs.

As used herein, the terms “progeny” and “progeny plant” refer to a plant generated from vegetative or sexual reproduction from one or more parent plants. In gynogenesis-mediated haploid induction, the haploid embryo on the female parent comprises female chromosomes to the exclusion of male chromosomes—thus it is not a progeny of the male haploid-inducing line. The haploid corn seed typically still has normal triploid endosperm that contains the male genome. The edited haploid progeny and subsequent edited doubled haploid plants and subsequent seed is not the only desired progeny. There is also the seed from the haploid inducer line itself, often carrying the Cas9 transgene, and subsequent plant and seed progeny of the haploid inducing plant. Both the haploid seed and the haploid inducer (self-pollination-derived) seed can be progeny. A progeny plant can be obtained by cloning or selfing a single parent plant, or by crossing two or more parental plants. For instance, a progeny plant can be obtained by cloning or selfing of a parent plant or by crossing two parental plants and include selfings as well as the F1 or F2 or still further generations. An F1 is a first-generation progeny produced from parents at least one of which is used for the first time as donor of a trait, while progeny of second generation (F2) or subsequent generations (F3, F4, and the like) are specimens produced from selfings, intercrosses, backcrosses, and/or other crosses of F1 s, F2 s, and the like. An F1 can thus be (and in some embodiments is) a hybrid resulting from a cross between two true breeding parents (i.e., parents that are true-breeding are each homozygous for a trait of interest or an allele thereof), while an F2 can be (and in some embodiments is) a progeny resulting from self-pollination of the F1 hybrids. The term “progeny” can in certain embodiments be used interchangeably with “offspring”, in particular when the plant or plant material is derived from sexual crossing of parent plants.

In certain embodiments, the plant is a crop plant, such as a cash crop or subsistence crop, such as food or non-food crops, including agriculture, horticulture, floriculture, or industrial crops. The term crop plant has its ordinary meaning as known in the art. By means of further guidance, and without limitation, a crop plant is a plant grown by humans for food and other resources, and can be grown and harvested extensively for profit or subsistence, typically in an agricultural setting or context.

In the context of the present invention, unless indicated otherwise, a “plant” may be of any species from the dicotyledon, monocotyledon, and gymnosperm plants. Non-limiting examples include Hordeum vulgare, Sorghum bicolor, Secale cereale, Triticale, Saccharum officinarium, Zea mays, Setaria italic, Oryza sativa, Oryza minuta, Oryza australiensis, Oryza alta, Triticum aestivum, Triticum durum, Hordeum bulbosum, Brachypodiurn distachyon, Hordeum marinum, Aegilops tauschii, Beta vulgaris, Helianthus annuus, Daucus glochidiatus, Daucus pusillus, Daucus muricatus, Daucus carota, Eucalyptus grandis, Erythranthe guttata, Genlisea aurea, Gossypium sp., Musa sp., Avena sp., Nicotiana sylvestris, Nicotiana tabacum, Nicotiana tomentosiformis, Solanum lycopersicum, Solanum tuberosum, Coffea canephora, Vitis vinifera, Cucumis sativus, Morus notabilis, Arabidopsis thaliana, Arabidopsis lyrata, Arabidopsis arenosa, Crucihimalaya himalaica, Crucihimalaya wallichii, Cardamine flexuosa, Lepidiurn virginicum, Capsella bursa-pastoris, Olmarabidopsis pumila, Arabis hirsuta, Brassica napus, Brassica oleracea, Brassica rapa, Brassica juncacea, Brassica nigra, Raphanus sativus, Eruca vesicaria sativa, Citrus sinensis, Jatropha curcas, Glycine max, and Populus trichocarpa. Preferably, a plant as used herein is of the genus Zea, preferably the species Zea mays, of the genus Sorghum, preferably the species Sorghum bicolor, or of the genus Brassica, preferably the species Brassica napus.

As used herein, “maize” refers to a plant of the species Zea mays, preferably Zea mays ssp mays.

As used herein, “sorghum” refers to a plant of the genus Sorghum, and includes without limitation Sorghum bicolor, Sorghum sudanense, Sorghum bicolor x Sorghum sudanense, Sorghum x almum (Sorghum bicolor x Sorghum halepense), Sorghum arundinaceum, Sorghum x drummondii, Sorghum halepense and/or Sorghum propinquum.

As used herein, the term “rapeseed” refers to a plant of the genus Brassica, and includes without limitation Brassica napus, preferably Brassica napus ssp napus. Rapeseed includes canola, Brassica oleracea, Brassica rapa, Brassica juncacea and/or Brassica nigra.

As used herein unless clearly indicated otherwise, the term “plant” intended to mean a plant at any developmental stage.

As used herein, the term “plant (part) population” may be used interchangeably with population of plants or plant parts. A plant (part) population preferably comprises a multitude of individual plants (or plant parts thereof), such as preferably at least 10, such as 20, 30, 40, 50, 60, 70, 80, or 90, more preferably at least 100, such as 200, 300, 400, 500, 600, 700, 800, or 900, even more preferably at least 1000, such as at least 10000 or at least 100000.

In certain embodiments, the plant population (or plant parts thereof) is a plant line, strain, or variety. In certain embodiments, the plant population (or plant parts thereof) is not a plant line, strain, or variety. In certain embodiments, the plant population (or plant parts thereof) is an inbred plant line, strain, or variety. In certain embodiments, the plant population (or plant parts thereof) is not an inbred plant line, strain, or variety. In certain embodiments, the plant population (or plant parts thereof) is an outbred plant line, strain, or variety. In certain embodiments, the plant population (or plant parts thereof) is not an outbred plant line, strain, or variety.

As used herein, the terms “phenotype,” “phenotypic trait” or “trait” refer to one or more traits of a plant or plant cell. The phenotype can be observable to the naked eye, or by any other means of evaluation known in the art, e.g., microscopy, biochemical analysis, or an electromechanical assay. In some cases, a phenotype is directly controlled by a single gene or genetic locus (i.e., corresponds to a “single gene trait”). In the case of haploid induction use of color markers, such as R Navajo, and other markers including transgenes visualized by the presences or absences of color within the seed evidence if the seed is an induced haploid seed. The use of R Navajo as a color marker and the use of transgenes is well known in the art as means to detect induction of haploid seed on the female plant. In other cases, a phenotype is the result of interactions among several genes, which in some embodiments also results from an interaction of the plant and/or plant cell with its environment.

The term “sequence” when used herein relates to nucleotide sequence(s), polynucleotide(s), nucleic acid sequence(s), nucleic acid(s), nucleic acid molecule, peptides, polypeptides and proteins, depending on the context in which the term “sequence” is used.

The terms “polynucleic acid”, “nucleotide sequence(s)”, “polynucleotide(s)”, “nucleic acid sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length. Nucleic acid sequences include DNA, cDNA, genomic DNA, RNA, synthetic forms and mixed polymers, both sense and antisense strands, or may contain non-natural or derivatized nucleotide bases, as will be readily appreciated by those skilled in the art.

When used herein, the term “polypeptide” or “protein” (both terms are used interchangeably herein) means a peptide, a protein, or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine. Peptides, oligopeptides and proteins may be termed polypeptides. The term polypeptide also refers to, and does not exclude, modifications of the polypeptide, e.g., glycosylation, acetylation, phosphorylation and the like. Such modifications are well described in basic texts and in more detailed monographs, as well as in the research literature.

The term “gene” when used herein refers to a polymeric form of nucleotides of any length, either ribonucleotides or desoxyribonucleotides. The term includes double- and single-stranded DNA and RNA. It also includes known types of modifications, for example, methylation, “caps”, substitutions of one or more of the naturally occurring nucleotides with an analog. Preferably, a gene comprises a coding sequence encoding the herein defined polypeptide. A “coding sequence” is a nucleotide sequence which is transcribed into mRNA and/or translated into a polypeptide when placed or being under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleic acid sequences or genomic DNA, while introns may be present as well under certain circumstances.

A used herein, the term “endogenous” refers to a gene or allele which is present in its natural genomic location. The term “endogenous” can be used interchangeably with “native”. This does not however exclude the presence of one or more nucleic acid differences with the wild-type allele, due to naturally occurring polymorphisms. In particular embodiments, the difference with a wild-type allele can be limited to less than 9 preferably less than 6, more particularly less than 3 nucleotide differences. More particularly, the difference with the wildtype sequence can be in only one nucleotide. A used herein, the term “endogenous” may refer to a gene or allele which has not been introduced into the plant (or its ancestry) by genetic engineering techniques or (artificial) mutagenesis. Naturally occurring variations/mutations may equally be considered endogenous. The term “endogenous” can be used interchangeably with “native” or “wild type”. Naturally occurring polymorphisms can all be considered endogenous, native, and/or wild type, in contrast to artificially introduced mutations or polymorphisms. Nevertheless, if a naturally occurring polymorphism (such as the naturally occurring ig mutations conferring haploid induction activity) has a particular phenotypic effect, such polymorphism may be considered a mutation in the context of the present invention. Non-naturally occurring polymorphisms or mutations, such as those introduced by random mutagenesis, may be considered exogenous, non-native, or genetically engineered.

The term “locus” (loci plural) means a specific place or places or a site on a chromosome where a genomic region of interest, for example a QTL, a gene or genetic marker, is found. A haplotype can be defined by the unique fingerprint of alleles at each marker within the specified window. As used herein, the term “allele” or “alleles” refers to one or more alternative forms, i.e. different nucleotide sequences, of a locus. Typically, an allele refers to alternative forms of various genetic units associated with different forms of a gene or of any kind of identifiable genetic element, which are alternative in inheritance because they are situated at the same locus in homologous chromosomes. In a diploid cell or organism, the two alleles of a given gene (or marker) typically occupy corresponding loci on a pair of homologous chromosomes.

A “marker” is a (means of finding a) position on a genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype (such as the ‘waxy’ phenotype). A DNA marker can be developed from genomic nucleotide sequence or from expressed nucleotide sequences (e.g., from a spliced RNA or a cDNA). Depending on the DNA marker technology, the marker may consist of complementary primers flanking the locus and/or complementary probes that hybridize to polymorphic alleles at the locus. The term marker locus is the locus (gene, sequence or nucleotide) that the marker detects. “Marker” or “molecular marker” or “marker locus” may also be used to denote a nucleic acid or amino acid sequence that is sufficiently unique to characterize a specific locus on the genome. Any detectable polymorphic trait can be used as a marker so long as it is inherited differentially and exhibits linkage disequilibrium with a phenotypic trait of interest.

Markers that detect genetic polymorphisms between members of a population are well-established in the art. Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected e.g. via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype. Haplotypes tend to be more informative (detect a higher level of polymorphism) than SNPs. A “marker allele”, alternatively an “allele of a marker locus”, can refer to one of a plurality of polymorphic nucleotide sequences found at a marker locus in a population. With regard to a SNP marker, allele refers to the specific nucleotide base present at that SNP locus in that individual plant.

“Marker assisted selection” (of MAS) is a process by which individual plants are selected based on marker genotypes. “Marker assisted counter-selection” is a process by which marker genotypes are used to identify plants that will not be selected, allowing them to be removed from a breeding program or planting. Marker assisted selection uses the presence of molecular markers, which are genetically linked to a particular locus or to a particular chromosome region (e.g. introgression fragment, transgene, polymorphism, mutation, etc), to select plants for the presence of the specific locus or region (introgression fragment, transgene, polymorphism, mutation, etc). For example, a molecular marker genetically linked to a genomic region of interest as defined herein, can be used to detect and/or select plants comprising the genomic region of interest. The closer the genetic linkage of the molecular marker to the locus (e.g. about 7 cM, 6 cM, 5 cM, 4 cM, 3 cM, 2 cM, 1 cM, 0.5 cM or less), the less likely it is that the marker is dissociated from the locus through meiotic recombination. Likewise, the closer two markers are linked to each other (e.g. within 7 or 5 cM, 4 cM, 3 cM, 2 cM, 1 cM or less) the less likely it is that the two markers will be separated from one another (and the more likely they will co-segregate as a unit). A marker “within 7 cM or within 5 cM, 3 cM, 2 cM, or 1 cM” of another marker refers to a marker which genetically maps to within the 7 cM or 5 cM, 3 cM, 2 cM, or 1 cM region flanking the marker (i.e. either side of the marker). Similarly, a marker within 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less of another marker refers to a marker which is physically located within the 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less, of the genomic DNA region flanking the marker (i.e. either side of the marker). “LOD-score” (logarithm (base 10) of odds) refers to a statistical test often used for linkage analysis in animal and plant populations. The LOD (“logarithm of odds”) score compares the likelihood of obtaining the test data if the two loci (molecular marker loci and/or a phenotypic trait locus) are indeed linked, to the likelihood of observing the same data purely by chance. Positive LOD scores favour the presence of linkage and a LOD score greater than 3.0 is considered evidence for linkage. A LOD score of +3 indicates 1000 to 1 odds that the linkage being observed did not occur by chance.

A centimorgan (“cM”) is a unit of measure of recombination frequency. One cM is equal to a 1% chance that a marker at one genetic locus will be separated from a marker at a second locus due to crossing over in a single generation.

“Physical distance” between loci (e.g. between molecular markers and/or between phenotypic markers) on the same chromosome is the actually physical distance expressed in bases or base pairs (bp), kilo bases or kilo base pairs (kb) or megabases or mega base pairs (Mb).

“Genetic distance” between loci (e.g. between molecular markers and/or between phenotypic markers) on the same chromosome is measured by frequency of crossing-over, or recombination frequency (RF) and is indicated in centimorgans (cM). One cM corresponds to a recombination frequency of 1%. If no recombinants can be found, the RF is zero and the loci are either extremely close together physically or they are identical. The further apart two loci are, the higher the RF.

A “marker haplotype” refers to a combination of alleles at a marker locus.

A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

A “marker probe” is a nucleic acid sequence or molecule that can be used to identify the presence of a marker locus, e.g., a nucleic acid probe that is complementary to a marker locus sequence, through nucleic acid hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e., genotype) the particular allele that is present at a marker locus.

The term “molecular marker” may be used to refer to a genetic marker or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g. SNP technology is used in the examples provided herein.

“Genetic markers” are nucleic acids that are polymorphic in a population and where the alleles of which can be detected and distinguished by one or more analytic methods, e.g., RFLP, AFLP, isozyme, SNP, SSR, and the like. The terms “molecular marker” and “genetic marker” are used interchangeably herein. The term also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also know for the detection of expressed sequence tags (ESTs) and SSR markers derived from EST sequences and randomly amplified polymorphic DNA (RAPD). Without limitation, screening may encompass or comprise sequencing, hybridization based methods (such as (dynamic) allele-specific hybridization, molecular beacons, SNP microarrays), enzyme based methods (such as PCR, KASP (Kompetitive Allele Specific PCR), RFLP, ALFP, RAPD, Flap endonuclease, primer extension, 5′-nuclease, oligonucleotide ligation assay), post-amplification methods based on physical properties of DNA (such as single strand conformation polymorphism, temperature gradient gel electrophoresis, denaturing high performance liquid chromatography, high-resolution melting of the entire amplicon, use of DNA mismatch-binding proteins, SNPlex, surveyor nuclease assay), etc.

The term “linked” or “closely linked”, in the present application, means that recombination between two linked loci occurs with a frequency of equal to or less than about 20% (i.e., are separated on a genetic map by not more than 20 cM). Put another way, the closely linked loci co-segregate at least 80% of the time. Marker loci are especially useful with respect to the subject matter of the current disclosure when they demonstrate a significant probability of co-segregation (linkage) with a desired trait. Closely linked loci such as a marker locus and a second locus can display an inter-locus recombination frequency of 20% or less, such as 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 20%, such as less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

“Linkage” refers to the tendency for alleles to segregate together more often than expected by chance if their transmission was independent. Typically, linkage refers to alleles on the same chromosome. Genetic recombination occurs with an assumed random frequency over the entire genome. Genetic maps are constructed by measuring the frequency of recombination between pairs of traits or markers. The closer the traits or markers are to each other on the chromosome, the lower the frequency of recombination, and the greater the degree of linkage. Traits or markers are considered herein to be linked if they generally co-segregate. A 1/100 probability of recombination per generation is defined as a genetic map distance of 1.0 centiMorgan (1.0 cM). The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype, such as the genomic region of interest as defined herein elsewhere. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

The genetic elements or genes located on a single chromosome segment are physically linked. In some embodiments, the two loci are located in close proximity such that recombination between homologous chromosome pairs does not occur between the two loci during meiosis with high frequency, e.g., such that linked loci co-segregate at least about 80% of the time, preferably at least 90% of the time, e.g., 91%, 92%, 93%, 94%, 95%, 96%, 97%, 98%, 99%, 99.5%, 99.75%, or more of the time. The genetic elements located within a chromosomal segment are also “genetically linked”, typically within a genetic recombination distance of less than or equal to 50 cM, e.g., about 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.75, 0.5, 0.25 cM or less. That is, two genetic elements within a single chromosomal segment undergo recombination during meiosis with each other at a frequency of less than or equal to about 50%, e.g., about 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.75%, 0.5%, 0.25% or less. “Closely linked” markers display a cross over frequency with a given marker of about 10% or less, e.g., 9%, 8%, 7%, 6%, 5%, 4%, 3%, 2%, 1%, 0.75%, 0.5%, 0.25% or less (the given marker locus is within about 10 cM of a closely linked marker locus, e.g., 9, 8, 7, 6, 5, 4, 3, 2, 1, 0.75, 0.5, 0.25 cM or less of a closely linked marker locus). Put another way, closely linked marker loci co-segregate at least about 80% of the time, such as at least 90% the time, e.g., 91%, 92%, 93%, 94%, 95%, 96%, 97%, 98%, 99%, 99.5%, 99.75%, or more of the time.

As used herein, the terms “introgression”, “introgressed” and “introgressing” refer to both a natural and artificial process whereby chromosomal fragments or genes of one species, variety or cultivar are moved into the genome of another species, variety or cultivar, by crossing those species. The process may optionally be completed by backcrossing to the recurrent parent. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. In any case, offspring comprising the desired allele can be repeatedly backcrossed to a line having a desired genetic background and selected for the desired allele, to result in the allele becoming fixed in a selected genetic background. The process of “introgressing” is often referred to as “backcrossing” when the process is repeated two or more times. “Introgression fragment” or “introgression segment” or “introgression region” refers to a chromosome fragment (or chromosome part or region) which has been introduced into another plant of the same or related species either artificially or naturally such as by crossing or traditional breeding techniques, such as backcrossing, i.e. the introgressed fragment is the result of breeding methods referred to by the verb “to introgress” (such as backcrossing). It is understood that the term “introgression fragment” never includes a whole chromosome, but only a part of a chromosome. The introgression fragment can be large, e.g. even three quarter or half of a chromosome, but is preferably smaller, such as about 15 Mb or less, such as about 10 Mb or less, about 9 Mb or less, about 8 Mb or less, about 7 Mb or less, about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less.

A genetic element, an introgression fragment, or a gene or allele conferring a trait as described herein is said to be “obtainable from” or can be “obtained from” or “derivable from” or can be “derived from” or “as present in” or “as found in” a plant or plant part as described herein elsewhere if it can be transferred from the plant in which it is present into another plant in which it is not present (such as a line or variety) using traditional breeding techniques without resulting in a phenotypic change of the recipient plant apart from the addition of the trait conferred by the genetic element, locus, introgression fragment, gene or allele as described herein. The terms are used interchangeably and the genetic element, locus, introgression fragment, gene, marker or allele can thus be transferred into any other genetic background lacking the trait. Not only pants comprising the genetic element, locus, introgression fragment, gene, or allele can be used, but also progeny/descendants from such plants which have been selected to retain the genetic element, locus, introgression fragment, gene, or allele, can be used and are encompassed herein. Whether a plant (or genomic DNA, cell or tissue of a plant) comprises the same genetic element, locus, introgression fragment, gene, or allele as obtainable from such plant can be determined by the skilled person using one or more techniques known in the art, such as phenotypic assays, whole genome sequencing, molecular marker analysis, trait mapping, chromosome painting, allelism tests and the like, or combinations of techniques. It will be understood that transgenic plants may also be encompassed.

As used herein the terms “genetic engineering”, “transformation” and “genetic modification” are all used herein as synonyms for the transfer of isolated and cloned genes into the DNA, usually the chromosomal DNA or genome, of another organism.

“Transgenic” or “genetically modified organisms” (GMOs) as used herein are organisms whose genetic material has been altered using techniques generally known as “recombinant DNA technology”. Recombinant DNA technology encompasses the ability to combine DNA molecules from different sources into one molecule ex vivo (e.g. in a test tube). This terminology generally does not cover organisms whose genetic composition has been altered by conventional cross-breeding or by “mutagenesis” breeding, as these methods predate the discovery of recombinant DNA techniques. “Non-transgenic” as used herein refers to plants and food products derived from plants that are not “transgenic” or “genetically modified organisms” as defined above.

“Transgene” or “chimeric gene” refers to a genetic locus comprising a DNA sequence, such as a recombinant gene, which has been introduced into the genome of a plant by transformation, such as Agrobacterium mediated transformation. A plant comprising a transgene stably integrated into its genome is referred to as “transgenic plant”.

As used herein, the term “homozygote” refers to an individual cell or plant having the same alleles at one or more or all loci. When the term is used with reference to a specific locus or gene, it means at least that locus or gene has the same alleles. As used herein, the term “homozygous” means a genetic condition existing when identical alleles reside at corresponding loci on homologous chromosomes. Accordingly, for diploid organisms, the two alleles are identical, for tetraploid organisms, the 4 alleles are identical, etc. As used herein, the term “heterozygote” refers to an individual cell or plant having different alleles at one or more or all loci. When the term is used with reference to a specific locus or gene, it means at least that locus or gene has different alleles. Accordingly, for diploid organisms, the two alleles are not identical, for tetraploid organisms, the 4 alleles are not identical (i.e. at least one allele is different than the other alleles), etc. As used herein, the term “heterozygous” means a genetic condition existing when different alleles reside at corresponding loci on homologous chromosomes. In certain embodiments, the proteins, genes, or coding sequences as described herein is/are homozygous. In certain embodiments, the proteins, genes, or coding sequences as described herein are heterozygous.

In certain embodiments, proteins, genes, or coding sequence alleles as described herein is/are homozygous. In certain embodiments, the proteins, genes, or coding sequence alleles as described herein are heterozygous. It will be understood that homozygosity or heterozygosity preferably relates to at least a gene, i.e. the locus comprising the gene (or coding sequence derived thereof, or protein encoded thereby). However, more specifically, homozygosity or heterozygosity may equally refer to a particular mutation, such as a mutation described herein. Accordingly, a particular mutation can be considered to be homozygous (i.e. all alleles carry the mutation), whereas for instance the remainder of the gene, coding sequence, or protein may comprise differences between alleles.

In certain embodiments, the mutation as defined herein is homozygous. Accordingly, in diploid plants the two alleles are identical (at least with respect to the particular mutation), in tetraploid plants the four alleles are identical, and in hexaploid plants the six alleles are identical with respect to the mutation or marker. In certain embodiments, the mutation/marker as defined herein is heterozygous. Accordingly, in diploid plants the two alleles are not identical, in tetraploid plants the four alleles are not identical (for instance only one, two, or three alleles comprise the specific mutation/marker), and in hexaploid plants the six alleles are not identical with respect to the mutation or marker (for instance only one, two, three, four or five alleles comprise the specific mutation/marker). Similar considerations apply in case of pseudopolyploid pants.

The term “haploid” refers to the state (of a plant or plant cell, organ, or tissue) of having the number of sets of chromosomes normally found in gametes, i.e. pollen or ovules (of this plant or plant cell, organ or tissue). Typically, haploid refers to half the amount of chromosomes normally found in somatic cells. Haploid cells (or plants) can have more than one set of chromosomes, in particular in case of polyploid plants. For instance a plant whose somatic cells are tetraploid (four sets of chromosomes), will produce gametes by meiosis that contain two sets of chromosomes. These gametes might still be called haploid even though they are numerically diploid. Accordingly, a haploid plant derived from a plant normally being tetraploid will comprise two sets of chromosomes. An alternative name for such plant is dihaploid. Similarly, a haploid plant derived from a plant normally being hexaploid will comprise three sets of chromosomes. An alternative name for such plant is trihaploid.

The terms “haploid inducer” and “haploid inductor” are used as synonyms herein and refer to a plant that is capable of producing fertilized seeds or embryos which have a haploid chromosome set from a crossing with a plant of the same genus, preferably a plant of the same species, which is not a haploid inducer. Mechanistically, haploid induction results from uniparental elimination of chromosomes after fertilization. Haploid induction is frequently a medium to low penetrance trait of the inducer line, so the resulting progeny, depending on the species or situation, may be either diploid (if no genome loss takes place) or haploid (if genome loss does indeed take place). Haploids can be selected by any suitable means known in the art (e.g. by means of markers, cytology, karyotyping, etc.). In certain embodiments, a haploid inducer as used herein is capable of producing at least 0.1% haploid offspring. In certain embodiments, a haploid inducer as used herein is capable of producing at least 0.5% haploid offspring. In certain embodiments, a haploid inducer as used herein is capable of producing at least 1% haploid offspring. In certain embodiments, a haploid inducer as used herein is capable of producing at least 2% haploid offspring. In certain embodiments, a haploid inducer as used herein is capable of producing at least 3% haploid offspring. In certain embodiments, a haploid inducer as used herein is capable of producing at least 4% haploid offspring. In certain embodiments, a haploid inducer as used herein is capable of producing at least 5% haploid offspring, such as at least 6%, or at least 7%. It will be understood that certain genes or proteins encoded thereby, in particular the (mutated) genes as described herein confer haploid inducer or induction activity or capability or are enhancers of haploid inducer or induction activity or capability. Accordingly, in certain embodiments, each such gene or protein product encoded thereby individually or combined confers haploid inducer/induction activity or capability of at least 0.1%, such as at least 0.5%, at least 1%, at least 2%, at least 3%, at least 4%, at least 5%, at least 6%, or at least 7%. In certain embodiments, the combined genes or protein products encoded thereby enhance haploid inducer/induction activity or capability by at least 0.1%, such as at least 0.5%, at least 1%, at least 2%, at least 3%, at least 4%, at least 5%, at least 6%, or at least 7% compared to the haploid induction rate of a plant comprising only one of such gene or protein product encoded thereby.

As used herein, the term “enhancer of haploid inducing capability or activity” refers to a (mutated) gene of protein encoded thereby which may or may not on its own confer haploid inducing activity, but which, when combined with another (mutated) gene or protein encoded thereby, increases the haploid inducing capability or activity compared to the single presence of the other (mutated) gene or protein encoded thereby. In certain embodiments, the increase in haploid offspring is at least 0.1%, such as at least 0.5%, at least 1%, at least 2%, at least 3%, at least 4%, at least 5%, at least 6%, or at least 7% (referring to the final (average) haploid induction rate of a plant comprising both (mutated) proteins). “Enhancing or increasing the haploid induction capability of a haploid inducer” or “mediating the property of an enhancer of the haploid induction capability of an a haploid inducer” means that by the use of the polynucleic acids encoding a mutated protein as described herein, the haploid induction rate of a haploid inducer can preferably be increased with at least 0.1%, 0.2%, 0.3%, 0.4%, 0.5%, 0.6%, 0.7%, 0.8% or 0.9%, preferably with at least 1%, 1.5%, 2%, 2.5%, 3%, 3.5%, 4%, 4.5% or 5%, more preferably with at least 6%, 7%, 8%, 9%, 10%, 15%, 20%, 30% or 50% (referring to the increase in induction rate compared to a single (mutated) protein). The number of fertilized seeds or embryos which have a haploid chromosome set and which have arisen from a crossing of the haploid inducer with a plant of the same genus (preferably, a plant of the same species) which is not a haploid inducer may thus be higher by at least 0.1%, 0.2%, 0.3%, 0.4%, 0.5%, 0.6%, 0.7%, 0.8% or 0.9%, preferably at least 1%, 1.5%, 2%, 2.5%, 3%, 3.5%, 4%, 4.5% or 5%, more preferably, at least 6%, 7%, 8%, 9%, 10%, 15%, 20%, 30% or 50%, than the number of haploid fertilized seeds or embryos which is achieved without the use of the nucleic acid as described herein.

The term “haploid induction rate” refers to the (average) percentage of haploid offspring which is or can be produced by a haploid inducer. In certain embodiments, each such gene or protein product encoded thereby individually confers or enhances haploid inducer/induction activity or capability by at least 0.1%, such as at least 0.5%, at least 1%, at least 2%, at least 3%, at least 4%, at least 5%, at least 6%, or at least 7%. The term “haploid induction rate” refers to the (average) percentage of haploid offspring which is or can be produced by a haploid inducer. In certain embodiments, each such combination of genes or protein products encoded thereby confers or enhances haploid inducer/induction activity or capability by at least 0.1%, such as at least 0.5%, at least 1%, at least 2%, at least 3%, at least 4%, at least 5%, at least 6%, or at least 7%. The term “haploid induction rate” refers to the (average) percentage of haploid offspring which is or can be produced by a haploid inducer.

The term “paternal haploid inducer” or “paternal haploid induction” refers to the male plant being the haploid inducer. Accordingly, after fertilization of a female non-haploid inducer plant with a paternal (i.e. male) haploid inducer plant, the chromosomes deriving from the male/paternal haploid inducer plant are lost. The resulting haploid plant therefore comprises only the female-derived chromosomes. This process of haploid inducting may also be referred to as gynogenesis. The term “paternal haploid induction rate” refers to the (average) percentage of haploid offspring which is or can be produced by a paternal haploid inducer.

The term “maternal haploid inducer” or “maternal haploid induction” refers to the female plant being the haploid inducer. Accordingly, after fertilization of a maternal (i.e. female) haploid inducer plant with male non-haploid inducer plant, the chromosomes deriving from the female/maternal haploid inducer plant are lost. The resulting haploid plant therefore comprises only the male-derived chromosomes. This process of haploid inducting may also be referred to as androgenesis. The term “maternal haploid induction rate” refers to the (average) percentage of haploid offspring which is or can be produced by a maternal haploid inducer.

The term “mutation” or “mutated” as used herein refers to a gene or protein product thereof which is altered or modified such that the function normally attributed to the gene or protein product thereof is altered, or alternatively such that the expression, stability, and/or activity normally associated with the gene or protein product thereof is altered. Typically, a mutation as referred to herein results in a phenotypic effect, such as haploid induction, as described herein elsewhere. It will be understood that a mutation in a gene or protein product thereof is referred to in comparison with a gene or protein product thereof not having such mutation, such as a wild type or endogenous gene or protein product thereof. Typically, a mutation refers to a modification at the DNA level, and includes changes in the genetics and/or epigenetics. An alteration in the genetics may include an insertion, a deletion, an introduction of a stop codon, a base change (e.g. transition or transversion), or an alteration in splice junctions. These alterations may arise in coding or non-coding regions (e.g. promoter regions, exons, introns or splice junctions) of the endogenous DNA sequence. For example, an alteration in the genetics may be the exchange (including insertions, deletions) of at least one nucleotide in the endogenous DNA sequence or in a regulatory sequence of the endogenous DNA sequence. If such a nucleotide exchange takes place in a promoter, for example, this may lead to an altered activity of the promoter, since, for example, cis-regulator elements are modified such that the affinity of a transcription factor to the mutated cis-regulatory elements is altered in comparison to the wild-type promoter, so that the activity of the promoter with the mutated cis-regulatory elements is increased or reduced, depending upon whether the transcription factor is a repressor or inductor, or whether the affinity of the transcription factor to the mutated cis-regulatory elements is intensified or weakened. If such a nucleotide exchange occurs, e.g., in an encoding region of the endogenous DNA sequence, this may lead to an amino acid exchange in the encoded protein, which may produce an alteration in the activity or stability of the protein, in comparison to the wild-type protein. An alteration in the epigenetics may take place via an altered methylation pattern of the DNA. In certain embodiments, a mutation as referred to herein relates to the insertion of one or more nucleotides in a gene. In certain embodiments, a mutation as referred to herein relates to the deletion of one or more nucleotides in a gene. In certain embodiments, the mutation as referred to herein relates to the deletion as well as the insertion of one or more nucleotides. In certain embodiments, certain nucleotide stretches, such as for instance encoding a particular protein domain are deleted. In certain embodiments, certain nucleotide stretches, such as for instance encoding a particular protein domain are deleted and replaced by nucleotide sequences encoding a different protein domain (such as for instance, the “GFP-tailswap” CENH3 mutants as described herein elsewhere, see for instance Kelliher et al. (2016). “Maternal haploids are preferentially induced by CENH3-tailswap transgenic complementation in maize.” Frontiers in plant science, 7, 414, incorporated herein by reference in its entirety). In certain embodiments, a mutation as referred to herein relates to the exchange of one or more nucleotides in a gene by different nucleotides. In certain embodiments, the mutation is a nonsense mutation (i.e. the mutation results in the generation of a stop codon in a protein encoding sequence). In certain embodiments, the mutation is a frameshift mutation (i.e. an insertion or deletion of one or more nucleotides (not equal to three or a product thereof) in a protein encoding sequence). In certain embodiments, the mutation results in a truncated protein product. In certain embodiments, the mutation results in an N-terminally truncated protein product. In certain embodiments, the mutation results in a C-terminally truncated protein product. In certain embodiments, the mutation results in an N-terminally and C-terminally truncated protein product. In certain embodiments, the mutation results in an altered splice site (such as an altered splice donor and/or splice acceptor site). In certain embodiments, the mutation is in an exon. In certain embodiments, the mutation is in an intron. In certain embodiments, the mutation is in a regulatory sequence, such as a promoter. In certain embodiments, the mutation results in a codon encoding a different amino acid. In certain embodiments, the mutation results in the insertion or deletion of one or more codons (i.e. nucleotide triplets). In certain embodiments, the mutation is a knockout mutation. Both frameshift and nonsense mutations can in certain embodiments be considered as knockout mutations, in particular if the mutation is present in an early exon. A knockout mutation as used herein preferably means that a functional gene product, such as a functional protein, is not produced anymore. In particular, frameshift and nonsense mutations will lead to premature termination of protein translation, such that a truncated protein will result, which often lacks the required stability and/or activity to perform the function naturally attributed to it. In certain embodiments, the mutation is a knockdown mutation. In contrast to a knockout mutation, a knockdown mutation results in a decreased activity, stability, and/or expression rate of the native functional gene product, such as a protein, and thereby ultimately in a decreased functionality. For instance, mutations in promoter regions affecting transcriptional activator binding (or other regulatory sequences), in particular reducing transcription rate, can be considered knockdown mutations. Also mutations negatively affecting protein stability (such as to increase ubiquitination and subsequent protein degradation) can be considered knockdown mutations). In addition, mutations negatively affecting protein activity (such as binding strength or enzymatic activity) can be considered knockdown mutations. It will be understood that the mutations described herein according to the invention confer haploid inducer or inducing activity or capability or enhance haploid inducer or inducing activity or capability, as described herein elsewhere. While mutation described herein may be non-naturally occurring, this need not necessarily be the case. For instance, as described herein elsewhere, for the indeterminate gametophyte (ig) gene, several naturally occurring mutations have been described which confer haploid inducing activity. In certain embodiments, the term “mutated protein” can be used interchangeably with “haploid inducing protein” or “haploid conferring protein” or the like. As used herein, a mutated protein, gene, allele, or coding sequence (i.e. polynucleic acid encoding for instance a protein) can be used interchangeably with a protein, gene, allele, or coding sequence conferring or enhancing haploid inducing activity or capability, as described herein elsewhere.

In certain embodiments, a wild type/endogenous allele is replaced by a mutated allele, preferably all wild type/endogenous alleles are replaced by a mutated allele. Replacement can be effected by any means known in the art, as also described herein elsewhere. Replacement, as used herein also includes (direct) mutagenesis of the wild type/endogenous allele(s) at its native genomic locus. Accordingly, in certain embodiments, a wild type/endogenous allele is mutated, as described herein elsewhere, preferably all wild type/endogenous alleles are mutated. The skilled person will understand that only one copy of a wild type/endogenous allele may be mutated and that homozygosity (if so desired) may be obtained by selfing and subsequent selection. In certain embodiments, a reduced number of wild type/endogenous alleles is present (i.e. the wild type/endogenous allele is heterozygous).

In certain embodiments, a wild type/endogenous allele is knocked out, preferably all wild type/endogenous alleles are knocked out, and a mutated allele is transgenically introduced, transiently or genomically integrated, preferably genomically integrated. In certain embodiments, a wild type/endogenous allele is knocked out, preferably all wild type/endogenous alleles are knocked out, and is transgenically replaced by a mutated allele (at the native genomic location of the wild type allele). The skilled person will understand that only one copy of a wild type/endogenous allele may be knocked out and that homozygosity (if so desired) may be obtained by selfing and subsequent selection.

In certain embodiments, the mutations as described herein, such as the ig mutations or the CENH3 mutations, are or result in amino acid substitutions (compared to the wild type or unmutated protein, gene, or coding sequence). In certain embodiments, the mutation is a point mutation. Preferably, the mutation is a missense mutation (i.e. the mutation results in a codon encoding a different amino acid). In certain embodiments one or more mutations are present. In certain embodiments, from 1 to 10 mutations are present. In certain embodiments, from 1 to 9 mutations are present. In certain embodiments, from 1 to 8 mutations are present. In certain embodiments, from 1 to 7 mutations are present. In certain embodiments, from 1 to 6 mutations are present. In certain embodiments, from 1 to 5 mutations are present. In certain embodiments, from 1 to 4 mutations are present. In certain embodiments, from 1 to 3 mutations are present. In certain embodiments, from 1 to 2 mutations are present. In certain embodiments, 1 mutation is present. In certain embodiments, from 1 to 10 amino acid substitutions are present in the mutated protein. In certain embodiments, from 1 to 9 amino acid substitutions are present in the mutated protein. In certain embodiments, from 1 to 8 amino acid substitutions are present in the mutated protein. In certain embodiments, from 1 to 7 amino acid substitutions are present in the mutated protein. In certain embodiments, from 1 to 6 amino acid substitutions are present in the mutated protein. In certain embodiments, from 1 to 5 amino acid substitutions are present in the mutated protein. In certain embodiments, from 1 to 4 amino acid substitutions are present in the mutated protein. In certain embodiments, from 1 to 3 amino acid substitutions are present in the mutated protein. In certain embodiments, from 1 to 2 amino acid substitutions are present in the mutated protein. In certain embodiments, 1 amino acid substitution is present in the mutated protein. In certain embodiments, from 1 to 10 point mutations, preferably missense mutations, are present in the mutated gene, allele, or coding sequence. In certain embodiments, from 1 to 9 point mutations, preferably missense mutations, are present in the mutated gene, allele, or coding sequence. In certain embodiments, from 1 to 8 point mutations, preferably missense mutations, are present in the mutated gene, allele, or coding sequence. In certain embodiments, from 1 to 7 point mutations, preferably missense mutations, are present in the mutated gene, allele, or coding sequence. In certain embodiments, from 1 to 6 point mutations, preferably missense mutations, are present in the mutated gene, allele, or coding sequence. In certain embodiments, from 1 to 5 point mutations, preferably missense mutations, are present in the mutated gene, allele, or coding sequence. In certain embodiments, from 1 to 4 point mutations, preferably missense mutations, are present in the mutated gene, allele, or coding sequence. In certain embodiments, from 1 to 3 point mutations, preferably missense mutations, are present in the mutated gene, allele, or coding sequence. In certain embodiments, from 1 to 2 point mutations, preferably missense mutations, are present in the mutated gene, allele, or coding sequence. In certain embodiments, 1 point mutation, preferably missense mutation, is present in the mutated gene, allele, or coding sequence.

The term “indeterminate gametophyte” or “ig” refers to the wild type indeterminate gametophyte gene or protein product encoded thereby. While it is appreciated that in literature the term indeterminate gametophyte may also refer to the mutated gene or phenotype thereof, i.e. haploid inducing, as used herein this term, unless otherwise explicitly specified, refers to the unmutated gene (or protein encoded thereby), i.e. the ig1 gene which does not confer or hardly confers haploid inducing activity. It will be understood that in this context, an ig1 gene which does not confer or hardly confers haploid inducing activity preferably refers to an ig1 gene for which the haploid induction rate is less than 1%, preferably less than 0.5%, more preferably less than 0.1%. In contrast, the term “mutant indeterminate gametophyte” refers to the mutant gene, such as the naturally occurring mutations, such as ig-O (ig1-O) or ig-mum (ig1-mum) which confers or enhances haploid inducing activity, as well as artificially generated mutations. At least three ig genes have been identified (see for instance US 2009/0151025, incorporated herein by reference in its entirety): ig1, ig2, and ig3. Preferably, according to the invention, the ig gene is ig1. Ig1 promotes the switch from proliferation to differentiation in the embryo sac. It is a negative regulator of cell proliferation in the adaxial side of leaves, and it regulates the formation of a symmetric lamina and the establishment of venation. Ig1 interacts directly with RS2 (rough sheath 2) to repress some knox homeobox genes (see Evans (2007) “The indeterminate gametophyte1 Gene of Maize Encodes a LOB Domain Protein Required for Embryo Sac and Leaf Development”; The Plant Cell; 19:46-62; incorporated herein by reference in its entirety. An alternative name of the ig1 gene is “LOB domain-containing protein 6”.

In a plant from the genus Zea, such as preferably Zea mays, the ig protein (i.e. the wild type ig) may have, comprise, or consist of a protein sequence as set forth in SEQ ID NO: 9 or 10, or a sequence which is at least 80%, preferably at least 90%, more preferably at least 95%, most preferably at least 98% identical to SEQ ID NO: 9 or 10. In a plant from the genus Zea, such as preferably Zea mays, the ig gene (i.e. the wild type ig) may have, comprise, or consist of a nucleic acid sequence as set forth in SEQ ID NO: 6, or a sequence which is at least 80%, preferably at least 90%, more preferably at least 95%, most preferably at least 98% identical to SEQ ID NO: 6. In a plant from the genus Zea, such as preferably Zea mays, the ig coding sequence (i.e. the wild type ig) may have, comprise, or consist of a nucleic acid sequence as set forth in SEQ ID NO: 7 or 8, or a sequence which is at least 80%, preferably at least 90%, more preferably at least 95%, most preferably at least 98% identical to SEQ ID NO: 7 or 8. The Zea mays ig protein, gene, or coding sequence is preferably the ig1 protein, gene, or coding sequence. In a plant from the genus Brassica, such as preferably Brassica napus, the ig protein (i.e. the wild type ig) may have, comprise, or consist of a protein sequence as set forth in SEQ ID NO: 29 or 32, or a sequence which is at least 80%, preferably at least 90%, more preferably at least 95%, most preferably at least 98% identical to SEQ ID NO: 29 or 32. In a plant from the genus Brassica, such as preferably Brassica napus, the ig gene (i.e. the wild type ig) may have, comprise, or consist of a nucleic acid sequence as set forth in SEQ ID NO: 27 or 30, or a sequence which is at least 80%, preferably at least 90%, more preferably at least 95%, most preferably at least 98% identical to SEQ ID NO: 27 or 30. In a plant from the genus Brassica, such as preferably Brassica napus, the ig coding sequence (i.e. the wild type ig) may have, comprise, or consist of a nucleic acid sequence as set forth in SEQ ID NO: 28 or 31, or a sequence which is at least 80%, preferably at least 90%, more preferably at least 95%, most preferably at least 98% identical to SEQ ID NO: 28 or 31. The Brassica napus ig protein, gene, or coding sequence is preferably the orthologue of the Zea mays ig (preferably ig1) protein, gene, or coding sequence. In a plant from the genus Sorghum, such as preferably Sorghum bicolor, the ig (preferably ig) protein (i.e. the wild type ig) may have, comprise, or consist of a protein sequence as set forth in SEQ ID NO: 23 or 26, or a sequence which is at least 80%, preferably at least 90%, more preferably at least 95%, most preferably at least 98% identical to SEQ ID NO: 23 or 26. In a plant from the genus Sorghum, such as preferably Sorghum bicolor, the ig gene (i.e. the wild type ig) may have, comprise, or consist of a nucleic acid sequence as set forth in SEQ ID NO: 21 or 24, or a sequence which is at least 80%, preferably at least 90%, more preferably at least 95%, most preferably at least 98% identical to SEQ ID NO: 21 or 24. In a plant from the genus Sorghum, such as preferably Sorghum bicolor, the ig coding sequence (i.e. the wild type ig) may have, comprise, or consist of a nucleic acid sequence as set forth in SEQ ID NO: 22 or 25, or a sequence which is at least 80%, preferably at least 90%, more preferably at least 95%, most preferably at least 98% identical to SEQ ID NO: 22 or 25. The Sorghum bicolor ig protein, gene, or coding sequence is preferably the orthologue of the Zea mays ig (preferably ig1) protein, gene, or coding sequence.

In certain embodiments, the indeterminate gametophyte gene encodes a protein which has a sequence which is at least 80% identical, preferably over its entire length, to a sequence as set forth in SEQ ID NO: 9, 10, 29, 32, 23, or 26. In certain embodiments, the indeterminate gametophyte gene encodes a protein which has a sequence which is at least 85% identical, preferably over its entire length, to a sequence as set forth in SEQ ID NO: 9, 10, 29, 32, 23, or 26. In certain embodiments, the indeterminate gametophyte gene encodes a protein which has a sequence which is at least 90% identical, preferably over its entire length, to a sequence as set forth in SEQ ID NO: 9, 10, 29, 32, 23, or 26. In certain embodiments, the indeterminate gametophyte gene encodes a protein which has a sequence which is at least 95% identical, preferably over its entire length, to a sequence as set forth in SEQ ID NO: 9, 10, 29, 32, 23, or 26. In certain embodiments, the indeterminate gametophyte gene encodes a protein which has a sequence which is at least 98% identical, preferably over its entire length, to a sequence as set forth in SEQ ID NO: 9, 10, 29, 32, 23, or 26. In certain embodiments, the indeterminate gametophyte gene encodes a protein which has a sequence which is at least 99% identical, preferably over its entire length, to a sequence as set forth in SEQ ID NO: 9, 10, 29, 32, 23, or 26. In certain embodiments, the indeterminate gametophyte gene encodes a protein which has a sequence which is identical to a sequence as set forth in SEQ ID NO: 9, 10, 29, 32, 23, or 26.

In certain embodiments, the indeterminate gametophyte gene encodes a protein which has a LOB domain having a sequence which is at least 80% identical to the sequence of the LOB domain of ig, preferably as set forth in SEQ ID NO: 9, 10, 29, 32, 23, or 26. In certain embodiments, the indeterminate gametophyte gene encodes a protein which has a LOB domain having a sequence which is at least 85% identical to the sequence of the LOB domain of ig, preferably as set forth in SEQ ID NO: 9, 10, 29, 32, 23, or 26. In certain embodiments, the indeterminate gametophyte gene encodes a protein which has a LOB domain having a sequence which is at least 90% identical to the sequence of the LOB domain of ig, preferably as set forth in SEQ ID NO: 9, 10, 29, 32, 23, or 26. In certain embodiments, the indeterminate gametophyte gene encodes a protein which has a LOB domain having a sequence which is at least 95% identical to the sequence of the LOB domain of ig, preferably as set forth in SEQ ID NO: 9, 10, 29, 32, 23, or 26. In certain embodiments, the indeterminate gametophyte gene encodes a protein which has a LOB domain having a sequence which is at least 98% identical to the sequence of the LOB domain of ig, preferably as set forth in 9, 10, 29, 32, 23, or 26. In certain embodiments, the indeterminate gametophyte gene encodes a protein which has a LOB domain having a sequence which is at least 99% identical to the sequence of the LOB domain of ig, preferably as set forth in SEQ ID NO: 9, 10, 29, 32, 23, or 26.

In certain embodiments, the indeterminate gametophyte gene encodes a protein which comprises a region having a sequence which is at least 80% identical to amino acids 30 to 145 of a sequence as set forth in SEQ ID NO: 9 or 10, or a corresponding region in SEQ ID NO: 23, 26, 29, or 31. In certain embodiments, the indeterminate gametophyte gene encodes a protein which comprises a sequence which is at least 85% identical to amino acids 30 to 145 of a sequence as set forth in SEQ ID NO: 9 or 10, or a corresponding region in SEQ ID NO: 23, 26, 29, or 31. In certain embodiments, the indeterminate gametophyte gene encodes a protein which comprises a sequence which is at least 90% identical to amino acids 30 to 145 of a sequence as set forth in SEQ ID NO: 9 or 10, or a corresponding region in SEQ ID NO: 23, 26, 29, or 31. In certain embodiments, the indeterminate gametophyte gene encodes a protein which comprises a sequence which is at least 95% identical to amino acids 30 to 145 of a sequence as set forth in SEQ ID NO: 9 or 10, or a corresponding region in SEQ ID NO: 23, 26, 29, or 31. In certain embodiments, the indeterminate gametophyte gene encodes a protein which comprises a sequence which is at least 98% identical to amino acids 30 to 145 of a sequence as set forth in SEQ ID NO: 9 or 10, or a corresponding region in SEQ ID NO: 23, 26, 29, or 31. In certain embodiments, the indeterminate gametophyte gene encodes a protein which comprises a sequence which is at least 99% identical to amino acids 30 to 145 of a sequence as set forth in SEQ ID NO: 9 or 10, or a corresponding region in SEQ ID NO: 23, 26, 29, or 31. It will be understood that sequence variants still maintain wild type ig functionality. In certain embodiments, ig is an orthologue of Zea mays ig, Sorghum bicolor ig, or Brassica napus ig. In certain embodiments, ig1 is an orthologue of Zea mays ig1, Sorghum bicolor ig1, or Brassica napus ig1.

In certain embodiments, the mutated ig gene or the ig gene conferring or enhancing haploid inducing activity or capability comprises an insertion of one or more nucleotides. In certain embodiments, the mutated ig coding sequence or the ig coding sequence conferring or enhancing haploid inducing activity or capability comprises an insertion of one or more nucleotides. In certain embodiments, the polynucleic acid encoding the mutated ig protein or the polynucleic acid encoding the ig protein conferring or enhancing haploid inducing activity or capability comprises an insertion of one or more nucleotides. In certain embodiments, the insertion is an insertion of 1 to 1000 nucleotides. In certain embodiments, the insertion is an insertion of 1 to 500 nucleotides. In certain embodiments, the insertion is an insertion of 1 to 300 nucleotides. In certain embodiments, the insertion is an insertion of 1 to 200 nucleotides. In certain embodiments, the insertion is an insertion of 10 to 1000 nucleotides. In certain embodiments, the insertion is an insertion of 10 to 500 nucleotides. In certain embodiments, the insertion is an insertion of 10 to 300 nucleotides. In certain embodiments, the insertion is an insertion of 10 to 200 nucleotides. In certain embodiments, the insertion is an insertion of 10 to 100 nucleotides. In certain embodiments, the insertion is an insertion of 10 to 100 nucleotides. In certain embodiments, the insertion is an insertion of 100 to 1000 nucleotides. In certain embodiments, the insertion is an insertion of 100 to 500 nucleotides. In certain embodiments, the insertion is an insertion of 100 to 300 nucleotides. In certain embodiments, the insertion is an insertion of 100 to 200 nucleotides. In certain embodiments, the insertion is an insertion of 200 to 1000 nucleotides. In certain embodiments, the insertion is an insertion of 200 to 500 nucleotides. In certain embodiments, the insertion is an insertion of 200 to 300 nucleotides. Preferably, the insertion is not a product of 3 nucleotides. The skilled person will understand that the presence of the insertion is compared to the unmutated or wild type, or the ig not conferring or enhancing haploid inducting activity or capability.

In certain embodiments, the insertion of one or more nucleotides is an insertion of one or more nucleotides in the LOB domain encoding region or sequence. In Zea mays, the LOB domain corresponds to amino acids 32 to 133, such as amino acids 32 to 133 of SEQ ID NO: 9 or 10. The skilled person can determine the corresponding positions delineating the LOB domain in orthologous ig genes or proteins.

In certain embodiments, the insertion of one or more nucleotides is an insertion of one or more nucleotides in the first protein encoding exon. In Zea mays, the first protein encoding exon is exon 2 (exon 1 being a 5′ UTR exon). In Zea mays, the first protein encoding exon corresponds to nucleotide positions 431 to 841 of the ig gene, such as nucleotide positions 431 to 841 of SEQ ID NO: 6. The skilled person can determine the corresponding positions delineating the first protein encoding exon in orthologous ig genes or proteins.

In certain embodiments, the insertion of one or more nucleotides is an insertion of one or more nucleotides in an intron, such as preferably the intron preceding the first protein encoding exon. In Zea mays, the intron preceding the first protein encoding exon is intron 1. The insertion of one or more nucleic acids in an intron preferably affects splicing, and results in a reduced (wild type) ig expression.

In certain embodiments, the mutated ig gene (or coding sequence) or the ig gene (or coding sequence) conferring or enhancing haploid inducing activity or capability corresponds to the ig1-O allele. In certain embodiments, the mutated ig gene or the ig gene conferring or enhancing haploid inducing activity or capability corresponds to the ig1-mum allele.

In certain embodiments, the mutated ig gene (or coding sequence) or the ig gene (or coding sequence) conferring or enhancing haploid inducing activity or capability comprises an insertion of one or more nucleic acids in an ig codon corresponding to a codon selected from codon 118, 119, or 120 of the wild type Zea mays ig coding sequence, such as set forth SEQ ID NO: 7 or 8.

In certain embodiments, the mutated ig gene (or coding sequence) or the ig gene (or coding sequence) conferring or enhancing haploid inducing activity or capability comprises an insertion of one or more nucleic acids in an ig codon corresponding to a codon selected from codon 191, 192, or 193 of the wild type Sorghum bicolor ig coding sequence, such as set forth in SEQ ID NO: 22.

In certain embodiments, the mutated ig gene (or coding sequence) or the ig gene (or coding sequence) conferring or enhancing haploid inducing activity or capability comprises an insertion of one or more nucleic acids in an ig codon corresponding to a codon selected from codon 143, 144, or 145 of the wild type Sorghum bicolor ig coding sequence, such as set forth in SEQ ID NO: 25.

In certain embodiments, the mutated ig gene (or coding sequence) or the ig gene (or coding sequence) conferring or enhancing haploid inducing activity or capability comprises an insertion of one or more nucleic acids in an ig codon corresponding to a codon selected from codon 94, 95 or 96 of the wild type Brassica napus ig coding sequence, such as set forth in SEQ ID NO: 28 or 31.

In certain embodiments, the mutated ig gene or the ig gene conferring or enhancing haploid inducing activity or capability comprises a frameshift mutation. In certain embodiments, the mutated ig coding sequence or the ig coding sequence conferring or enhancing haploid inducing activity or capability comprises a frameshift mutation. In certain embodiments, the polynucleic acid encoding the mutated ig protein or the polynucleic acid encoding the ig protein conferring or enhancing haploid inducing activity or capability comprises a frameshift mutation. A frameshift mutation is an insertion or deletion of one or more nucleotides which is not a product of 3 nucleotides. Preferably, a frameshift mutation is an insertion or deletion of 1 or 2 nucleotides. The skilled person will understand that the presence of the frameshift mutation is compared to the unmutated or wild type, or the ig not conferring or enhancing haploid inducting activity or capability.

In certain embodiments, the mutated ig gene or the ig gene conferring or enhancing haploid inducing activity or capability comprises a nonsense mutation. In certain embodiments, the mutated ig coding sequence or the ig coding sequence conferring or enhancing haploid inducing activity or capability comprises a nonesense mutation. In certain embodiments, the polynucleic acid encoding the mutated ig protein or the polynucleic acid encoding the ig protein conferring or enhancing haploid inducing activity or capability comprises a nonsense mutation. A nonsense mutation is a mutation in which an amino acid encoding codon is mutated to a stop codon. The skilled person will understand that the presence of the nonsense mutation is compared to the unmutated or wild type, or the ig not conferring or enhancing haploid inducting activity or capability.

In certain embodiments, the mutated ig gene or the ig gene conferring or enhancing haploid inducing activity or capability comprises a point mutation. In certain embodiments, the mutated ig coding sequence or the ig coding sequence conferring or enhancing haploid inducing activity or capability comprises a point mutation. In certain embodiments, the polynucleic acid encoding the mutated ig protein or the polynucleic acid encoding the ig protein conferring or enhancing haploid inducing activity or capability comprises a point mutation. A point mutation is a substitution of 1 nucleotide. Preferably, the point mutation is a missense mutation (i.e. a mutation in a codon as a result of which a different codon arises, which encodes a different amino acid). The skilled person will understand that the presence of the point mutation is compared to the unmutated or wild type, or the ig not conferring or enhancing haploid inducting activity or capability.

In certain embodiments, the mutated ig gene or the ig gene conferring or enhancing haploid inducing activity or capability comprises a knockout mutation. In certain embodiments, the mutated ig coding sequence or the ig coding sequence conferring or enhancing haploid inducing activity or capability comprises a knockout mutation. In certain embodiments, the polynucleic acid encoding the mutated ig protein or the polynucleic acid encoding the ig protein conferring or enhancing haploid inducing activity or capability comprises a knockout mutation. The skilled person will understand that the presence of the knockout mutation is compared to the unmutated or wild type, or the ig not conferring or enhancing haploid inducting activity or capability.

In certain embodiments, the mutated ig gene or the ig gene conferring or enhancing haploid inducing activity or capability comprises a knockdown mutation. In certain embodiments, the mutated ig coding sequence or the ig coding sequence conferring or enhancing haploid inducing activity or capability comprises a knockdown mutation. In certain embodiments, the polynucleic acid encoding the mutated ig protein or the polynucleic acid encoding the ig protein conferring or enhancing haploid inducing activity or capability comprises a knockdown mutation. The skilled person will understand that the presence of the knockout mutation is compared to the unmutated or wild type, or the ig not conferring or enhancing haploid inducting activity or capability. The skilled person will understand that instead of a knockdown mutation the same effect can be achieved for instance by RNAi (e.g. siRNA, shRNA) or by using site directed nucleases, such as RNA specific CRISPR/Cas systems, as described herein elsewhere.

In certain embodiments, the (wild type) ig gene, mRNA, and/or protein has a reduced expression or transcription (rate), a reduced stability, and/or a reduced activity.

As used herein, “reducing the expression (rate)” or “reduction in the expression rate” or “suppression of the expression” “reduced expression (rate)” or “repression” or a comparable phrase in certain embodiments means a reduction in the expression level or rate of a nucleotide or protein sequence by more than 10%, 15%, 20%, 25% or 30%, preferably by more than 40%, 45%, 50%, 55%, 60% or 65%, more preferably by more than 70%, 75%, 80%, 85%, 90%, 92%, 94%, 96% or 98% in comparison to the specified reference, such as a plant not comprising the genetic or otherwise modifications according to the invention as described herein elsewhere, or a reference plant (such as BL73 for maize). However, it may also mean that the expression rate of a nucleotide sequence or protein is reduced by 100%. The reduction in the expression rate preferably leads to a change of the phenotype of a plant in which the expression rate is reduced. In the context of the present invention, an altered phenotype may be the enhanced induction capability of a haploid inductor.

“Reduction in the transcription rate” or “reduced transcription rate” or a comparable phrase in certain embodiments means a reduction in the transcription rate of a nucleotide sequence by more than 10%, 15%, 20%, 25% or 30%, preferably by more than 40%, 45%, 50%, 55%, 60% or 65%, more preferably by more than 70%, 75%, 80%, 85%, 90%, 92%, 94%, 96% or 98% in comparison to the specified reference, such as a plant not comprising the genetic or otherwise modifications according to the invention as described herein elsewhere, or a reference plant (such as BL73 for maize). However, it may also mean that the transcription rate of a nucleotide sequence is reduced by 100%. The reduction in the transcription rate preferably leads to a change of the phenotype of a plant in which the transcription rate is reduced. In the context of the present invention, an altered phenotype may be the enhanced induction capability of a haploid inductor.

As used herein, “reduced (protein) activity” refers to reduced activity of about at least 10%, preferably at least 30%, more preferably at least 50%, such as at least 20%, 40%, 60%, 80% or more, such as at least 85%, at least 90%, at least 95%, or more. Activity is (substantially) absent or eliminated if activity is reduced at least 80%, preferably at least 90%, more preferably at least 95%. In certain embodiments, activity is (substantially) absent, if no activity, in particular the wild type or native protein activity, can be detected. (Protein) activity levels can be determined by any means known in the art, depending on the type of protein, such as by standard detection methods, including for instance enzymatic assays (for enzymes), transcription assays (for transcription factors), assays to analyse a phenotypic output, etc. Activity may be compared to a reference as defined above.

As used herein, “reduced stability” may refer to reduced protein stability or reduced RNA, such as mRNA stability. Stability of proteins or RNA can be determined by means known in the art, such as determination of protein/RNA half-life. Reduced protein or RNA stability in certain embodiments means a reduction of stability of about at least 10%, preferably at least 30%, more preferably at least 50%, such as at least 20%, 40%, 60%, 80% or more, such as at least 85%, at least 90%, or at least 95. Stability may be compared to a reference as defined above.

In certain embodiments, the mutated ig protein or the ig protein conferring or enhancing haploid inducing activity or capability comprises an insertion of one or more amino acids. In certain embodiments, the insertion is an insertion of 1 to 350 amino acids. In certain embodiments, the insertion is an insertion of 1 to 250 amino acids. In certain embodiments, the insertion is an insertion of 1 to 150 amino acids. In certain embodiments, the insertion is an insertion of 1 to 50 amino acids. In certain embodiments, the insertion is an insertion of 10 to 350 amino acids. In certain embodiments, the insertion is an insertion of 10 to 250 amino acids. In certain embodiments, the insertion is an insertion of 10 to 150 amino acids. In certain embodiments, the insertion is an insertion of 10 to 50 amino acids. In certain embodiments, the insertion is an insertion of 50 to 350 amino acids. In certain embodiments, the insertion is an insertion of 50 to 250 amino acids. In certain embodiments, the insertion is an insertion of 50 to 150 amino acids. In certain embodiments, the insertion is an insertion of 100 to 350 amino acids. In certain embodiments, the insertion is an insertion of 100 to 250 amino acids. In certain embodiments, the insertion is an insertion of 100 to 150 amino acids. The skilled person will understand that the presence of the insertion is compared to the unmutated or wild type, or the ig not conferring or enhancing haploid inducting activity or capability.

In certain embodiments, the mutated ig protein or the ig protein conferring or enhancing haploid inducing activity or capability comprises an insertion of one or more amino acids and/or substitution of one or more amino acids in a region corresponding to amino acid residues 110 to 130 of the wild type Zea mays ig protein, such as set forth in SEQ ID NO: 9 or 10 In certain embodiments, the mutated ig protein or the ig protein conferring or enhancing haploid inducing activity or capability comprises an insertion of one or more amino acids and/or substitution of one or more amino acids in a region corresponding to amino acid residues 183 to 203 of the wild type Sorghum bicolor ig protein, such as set forth in SEQ ID NO: 23.

In certain embodiments, the mutated ig protein or the ig protein conferring or enhancing haploid inducing activity or capability comprises an insertion of one or more amino acids and/or substitution of one or more amino acids in a region corresponding to amino acid residues 135 to 155 of the wild type Sorghum bicolor ig protein, such as set forth in SEQ ID NO: 26.

In certain embodiments, the mutated ig protein or the ig protein conferring or enhancing haploid inducing activity or capability comprises an insertion of one or more amino acids and/or substitution of one or more amino acids in a region or corresponding to amino acid residues 86 to 106 of the wild type Brassica napus ig protein, such as set forth in SEQ ID NO: 29 or 32.

In certain embodiments, the mutated ig protein or the ig protein conferring or enhancing haploid inducing activity or capability comprises an insertion of one or more amino acids and/or substitution of one or more amino acids in a region corresponding to amino acid residues 116 to 120, preferably 117 to 119 of the wild type Zea mays ig protein, such as set forth in SEQ ID NO: 9 or 10

In certain embodiments, the mutated ig protein or the ig protein conferring or enhancing haploid inducing activity or capability comprises an insertion of one or more amino acids and/or substitution of one or more amino acids in a region corresponding to amino acid residues 189 to 193, preferably 190 to 192 of the wild type Sorghum bicolor ig protein, such as set forth in SEQ ID NO: 23.

In certain embodiments, the mutated ig protein or the ig protein conferring or enhancing haploid inducing activity or capability comprises an insertion of one or more amino acids and/or substitution of one or more amino acids in a region corresponding to amino acid residues 141 to 145, preferably 142 to 144 of the wild type Sorghum bicolor ig protein, such as set forth in SEQ ID NO: 26.

In certain embodiments, the mutated ig protein or the ig protein conferring or enhancing haploid inducing activity or capability comprises an insertion of one or more amino acids and/or substitution of one or more amino acids in a region or corresponding to amino acid residues 92 to 96, preferably 93 to 95 of the wild type Brassica napus ig protein, such as set forth in SEQ ID NO: 29 or 32.

In certain embodiments, the mutated ig protein or the ig protein conferring or enhancing haploid inducing activity or capability is a truncated ig protein. In certain embodiments, the mutated ig protein or the ig protein conferring or enhancing haploid inducing activity or capability is a C-terminally truncated ig protein (i.e. the mutated protein comprises only the N-terminal part, such as the LOB domain).

In certain embodiments, the mutated ig protein or the ig protein conferring or enhancing haploid inducing activity or capability consists of a protein sequence corresponding to amino acid residues 1 to 116, 1 to 117, 1 to 118, 1 to 119, or 1 to 120, preferably 1 to 117, 1 to 118, or 1 to 119 of the wild type Zea mays ig protein, such as set forth in SEQ ID NO: 9 or 10

In certain embodiments, the mutated ig protein or the ig protein conferring or enhancing haploid inducing activity or capability consists of a protein sequence corresponding to amino acid residues 1 to 189, 1 to 190, 1 to 191, 1 to 192, or 1 to 193, preferably 1 to 190, 1 to 191, or 1 to 192 of the wild type Sorghum bicolor ig protein, such as set forth in SEQ ID NO: 23.

In certain embodiments, the mutated ig protein or the ig protein conferring or enhancing haploid inducing activity or capability consists of a protein sequence corresponding to amino acid residues 1 to 141, 1 to 142, 1 to 143, 1 to 144, or 1 to 145, preferably 1 to 142, 1 to 143, or 1 to 144 of the wild type Sorghum bicolor ig protein, such as set forth in SEQ ID NO: 26.

In certain embodiments, the mutated ig protein or the ig protein conferring or enhancing haploid inducing activity or capability consists of a protein sequence corresponding to amino acid residues 1 to 92, 1 to 93, 1 to 94, 1 to 95, or 1 to 96, preferably 1 to 93, 1 to 94, or 1 to 95 of the wild type Brassica napus ig protein, such as set forth in SEQ ID NO: 29 or 32.

In certain embodiments, the mutated ig protein or the ig protein conferring or enhancing haploid inducing activity or capability does not comprise a protein sequence corresponding to amino acid residues 117 to 260, 118 to 260, 119 to 260, 120 to 260, or 121 to 260, preferably 118 to 260, 119 to 260, or 120 to 260, of the wild type Zea mays ig protein, such as set forth in SEQ ID NO: 9 or 10.

In certain embodiments, the mutated ig protein or the ig protein conferring or enhancing haploid inducing activity or capability does not comprise a protein sequence corresponding to amino acid residues 190 to 332, 1 to 191 to 332, 192 to 332, 193 to 332, or 194 to 332, preferably 191 to 332, 192 to 332, or 193 to 332 of the wild type Sorghum bicolor ig protein, such as set forth in SEQ ID NO: 23.

In certain embodiments, the mutated ig protein or the ig protein conferring or enhancing haploid inducing activity or capability does not comprise a protein sequence corresponding to amino acid residues 142 to 308, 143 to 308, 144 to 308, 145 to 308, or 146 to 308, preferably 143 to 308, 144 to 308, or 145 to 308 of the wild type Sorghum bicolor ig protein, such as set forth in SEQ ID NO: 26.

In certain embodiments, the mutated ig protein or the ig protein conferring or enhancing haploid inducing activity or capability does not comprise a protein sequence corresponding to amino acid residues 93 to 202, 94 to 202, 95 to 202, 96 to 202, or 97 to 202, preferably 94 to 202, 95 to 202, or 96 to 202 of the wild type Brassica napus ig protein, such as set forth in SEQ ID NO: 29 or 32.

In a plant from the genus Zea, such as preferably Zea mays, the mutated ig protein or the ig protein conferring or enhancing haploid inducing activity or capability may have, comprise, or consist of a protein sequence as set forth in SEQ ID NO: 4 or 5, or a sequence which is at least 80%, preferably at least 90%, more preferably at least 95%, most preferably at least 98% identical to SEQ ID NO: 4 or 5. In a plant from the genus Zea, such as preferably Zea mays, the mutated ig gene or the ig gene conferring or enhancing haploid inducing activity or capability may have, comprise, or consist of a nucleic acid sequence as set forth in SEQ ID NO: 1, or a sequence which is at least 80%, preferably at least 90%, more preferably at least 95%, most preferably at least 98% identical to SEQ ID NO: 1. In a plant from the genus Zea, such as preferably Zea mays, the mutated ig coding sequence or the ig coding sequence conferring or enhancing haploid inducing activity or capability may have, comprise, or consist of a nucleic acid sequence as set forth in SEQ ID NO: 2 or 3, or a sequence which is at least 80%, preferably at least 90%, more preferably at least 95%, most preferably at least 98% identical to SEQ ID NO: 2 or 3. The mutated Zea mays ig protein, gene, or coding sequence is preferably the ig1 protein, gene, or coding sequence.

In certain embodiments, the mutated ig gene or allele or the ig gene or allele conferring or enhancing haploid inducing activity or capability encodes a protein which has a sequence which is at least 80% identical, preferably over its entire length, to a sequence as set forth in SEQ ID NO: 4 or 5. In certain embodiments, the mutated ig gene or allele or the ig gene or allele conferring or enhancing haploid inducing activity or capability encodes a protein which has a sequence which is at least 85% identical, preferably over its entire length, to a sequence as set forth in SEQ ID NO: 4 or 5. In certain embodiments, the mutated ig gene or allele or the ig gene or allele conferring or enhancing haploid inducing activity or capability encodes a protein which has a sequence which is at least 90% identical, preferably over its entire length, to a sequence as set forth in SEQ ID NO: 4 or 5. In certain embodiments, the mutated ig gene or allele or the ig gene or allele conferring or enhancing haploid inducing activity or capability encodes a protein which has a sequence which is at least 95% identical, preferably over its entire length, to a sequence as set forth in SEQ ID NO: 4 or 5. In certain embodiments, the mutated ig gene or allele or the ig gene or allele conferring or enhancing haploid inducing activity or capability encodes a protein which has a sequence which is at least 98% identical, preferably over its entire length, to a sequence as set forth in SEQ ID NO: 4 or 5. In certain embodiments, the mutated ig gene or allele or the ig gene or allele conferring or enhancing haploid inducing activity or capability encodes a protein which has a sequence which is at least 99% identical, preferably over its entire length, to a sequence as set forth in SEQ ID NO: 4 or 5. In certain embodiments, the mutated ig gene or allele or the ig gene or allele conferring or enhancing haploid inducing activity or capability encodes a protein which has a sequence which is identical to a sequence as set forth in SEQ ID NO: 4 or 5.

The term “centromere protein” refers to any protein associated with the centromere. These can be proteins associated with DNA at centromeric regions, such as centromeric histone proteins (e.g. CENH3). The term “kinetochore protein” refers to any protein associated with the kinetochore. These can be proteins which are present in the kinetochore, preferably excluding microtubular proteins such as tubulin. In certain embodiments, the centromere or kinetochore protein is a histone protein. In certain embodiments, the centromere or kinetochore protein is not a histone protein. In certain embodiments, the centromere or kinetochore protein is a CENP. It will be understood that in the context of the present invention the mutated centromere or kinetochore protein confers or enhances haploid inducing activity. In certain embodiments, the centromere or kinetochore protein is selected from CENH3 or any centromere or kinetochore interacting directly or indirectly with CENH3, preferably interacting directly with CENH3. In certain embodiments, the centromere or kinetochore protein is selected from CENH3, CENP-C, KNL2, SCM3, SAD2 and SIM3.

As used herein, “CENP-C” or “CENPC” refers to centromere protein C. By means of example, and without limitation, Zea mays CENP-C can have an amino acid sequence as set forth in NCBI Reference Sequence XP_008656649.1 (SEQ ID NO: 36). Sorghum bicolor CENP-C can have an amino acid sequence as set forth GenBank accession number AAU04623.1 (SEQ ID NO: 38). The skilled person will readily be able to identify orthologues in different plant species. Mutants of CENP-C conferring haploid inducing activity have been described for instance in Wang, N., & Dawe, R. K. (2018). “Centromere size and its relationship to haploid formation in plants.” Molecular plant, 11(3), 398-406, and WO2017058022A1, which are incorporated herein by reference in their entirety. A nucleic acid molecule encoding a CENP-C protein may be selected from the group consisting of:
i) a nucleic acid molecule having the coding sequence of SEQ ID NO: 35 or 37;
ii) a nucleic acid molecule having the coding sequence which is 80%, 85%, 90%, 92%, 94%, 96%, 98% or 99% identical to the sequence of SEQ ID NO: 35 or 37;
iii) a nucleic acid molecule encoding a protein having the amino acid sequence of SEQ ID NO: 36 or 38; or
iv) a nucleic acid molecule encoding a protein having an amino acid sequence which is 80%, 85%, 90%, 92%, 94%, 96%, 98% or 99% identical to the sequence of SEQ ID NO: 36 or 38.

As used herein, “KNL2” refers to Kinetochore-associated protein KNL-2 homolog or alternatively kinetochore null2. By means of example, and without limitation, Arabidopsis thaliana KNL2 can have an amino acid sequence as set forth in UniProtKB/Swiss-Prot accession number F4KCE9.1 (SEQ ID NO: 40). The skilled person will readily be able to identify orthologues in different plant species. Mutants of KNL2 conferring haploid inducing activity have been described for instance in Sandmann et al. (2017) “Targeting of Arabidopsis KNL2 to Centromeres Depends on the Conserved CENPC-k Motif in Its C Terminus” Plant Cell, 29(1):144-155, and US 2019/0075744 A1, which are incorporated herein by reference in their entirety. A nucleic acid molecule encoding a KNL2 protein may be selected from the group consisting of:
i) a nucleic acid molecule having a nucleotide sequence of SEQ ID NO: 41, 43, 45 or 47 or a nucleotide sequence which is 80%, 85%, 90%, 92%, 94%, 96%, 98% or 99% identical to the sequence of SEQ ID NO: 41, 43, 45 or 47;
ii) a nucleic acid molecule having the coding sequence of SEQ ID NO: 39 or a coding sequence which is 80%, 85%, 90%, 92%, 94%, 96%, 98% or 99% identical to the sequence of SEQ ID NO: 39;
iii) a nucleic acid molecule encoding a protein having the amino acid sequence of SEQ ID NO: 40, 42, 44, 46 or 48; or
iv) a nucleic acid molecule encoding a protein having an amino acid sequence which is 80%, 85%, 90%, 92%, 94%, 96%, 98% or 99% identical to the sequence of SEQ ID NO: 40, 42, 44, 46 or 48.

As used herein, “Scm3” refers to suppressor of chromosome missegregation protein 3, which was originally identified in Saccharomyces cerevisiae see for instance https://www.yeastgenome.org/locus/5000002298) (SEQ ID NO: 50). It is a homologue of HJURP. Scm3 is a chaperone protein for CENH3. A nucleic acid molecule encoding a Scm3 protein may be selected from the group consisting of:
i) a nucleic acid molecule having the coding sequence of SEQ ID NO: 49 or a coding sequence which is 80%, 85%, 90%, 92%, 94%, 96%, 98% or 99% identical to the sequence of SEQ ID NO: 49;
ii) a nucleic acid molecule encoding a protein having the amino acid sequence of SEQ ID NO: 50 or an amino acid sequence which is 80%, 85%, 90%, 92%, 94%, 96%, 98% or 99% identical to the sequence of SEQ ID NO: 50.

As used herein, “SAD2” refers ‘Sensitive to ABA (abscisic acid) and Drought2’ which was described in Verslues et al. (2006). Mutation of SAD2, an importin β-domain protein in Arabidopsis, alters abscisic acid sensitivity. The Plant Journal, 47(5), 776-787.). SAD2 encodes an importin beta-domain family protein likely to be involved in nuclear transport. SAD2 was expressed at a low level in all tissues examined except flowers, but SAD2 expression was not inducible by ABA or stress. Subcellular localization of GFP-tagged SAD2 showed a predominantly nuclear localization, consistent with a role for SAD2 in nuclear transport. SAD2 is in the same pathway as two transcription factors (GLABROUS1 (GL1) and GLABRA3 (GL3)). Recent publication demonstrated that mutant sad2 gene affects haploid induction in plants (EP 3 794 939 A1). A nucleic acid molecule encoding a SAD2 protein may be selected from the group consisting of:
i) a nucleic acid molecule having the coding sequence of SEQ ID NO: 51 or a coding sequence which is 80%, 85%, 90%, 92%, 94%, 96%, 98% or 99% identical to the sequence of SEQ ID NO: 51;
ii) a nucleic acid molecule encoding a protein having the amino acid sequence of any one of SEQ ID NO: 52-70, or an amino acid sequence which is 80%, 85%, 90%, 92%, 94%, 96%, 98% or 99% identical to the sequence of any one of SEQ ID NO: 52-70.

As used herein “SIM3” refers to NASP-related protein sim3. SIM3 is a Histone H3 and H3-like CENP-A-specific chaperone. SIM3 promotes delivery and incorporation of CENP-A in centromeric chromatin, probably by escorting nascent CENP-A to CENP-A chromatin assembly factors. It is required for central core silencing and normal chromosome segregation.

As used herein, “CENH3” refers to centromere specific histone H3. An alternative name is CENPA or CENP-A (centromere protein A). CENH3 is a centromere protein which contains a histone H3 related histone fold domain that is required for targeting to the centromere. Centromere protein A is proposed to be a component of a modified nucleosome or nucleosome-like structure in which it replaces 1 or both copies of conventional histone H3 in the (H3-H4)2 tetrameric core of the nucleosome particle. The protein is a replication-independent histone that is a member of the histone H3 family. In Arabidopsis thaliana, CENH3 may have a protein sequence as set forth in SEQ ID NO: 12. In Zea mays, CENH3 may have a protein sequence as set forth in SEQ ID NO: 14. In Brassica napus, CENH3 may have a protein sequence as set forth in SEQ ID NO: 16. In Sorghum bicolor, CENH3 may have a protein sequence as set forth in SEQ ID NO: 18. Accordingly, in certain embodiments, the CENH3 gene encodes a protein which has a sequence which is at least 80% identical, preferably over its entire length, to a sequence as set forth in SEQ ID NO: 12, 14, 16, or 18. In certain embodiments, the CENH3 gene encodes a protein which has a sequence which is at least 85% identical, preferably over its entire length, to a sequence as set forth in SEQ ID NO: 12, 14, 16, or 18. In certain embodiments, the CENH3 gene encodes a protein which has a sequence which is at least 90% identical, preferably over its entire length, to a sequence as set forth in SEQ ID NO: 12, 14, 16, or 18. In certain embodiments, the CENH3 gene encodes a protein which has a sequence which is at least 95% identical, preferably over its entire length, to a sequence as set forth in SEQ ID NO: 12, 14, 16, or 18. In certain embodiments, the CENH3 gene encodes a protein which has a sequence which is at least 98% identical, preferably over its entire length, to a sequence as set forth in SEQ ID NO: 12, 14, 16, or 18. In certain embodiments, the CENH3 gene encodes a protein which has a sequence which is at least 99% identical, preferably over its entire length, to a sequence as set forth in SEQ ID NO: 12, 14, 16, or 18. In certain embodiments, CENH3 is an orthologue of Zea mays CENH3, Sorghum bicolor CENH3, or Brassica napus CENH3.

In certain embodiments, the mutated CENH3 protein or the CENH3 protein conferring or enhancing haploid inducing activity or capability comprises one or more mutated amino acids, preferably one or more amino acid substitutions, in one or more of the N-terminal domain, the αN-helix, the α1-helix, the loop 1 domain, the α2-helix, the loop 2 domain, the α3-helix, or the C-terminal domain of CENH3, such as specified in Table 1.

TABLE 1
Protein domains of CENH3

| | | | | | | CATD | | | | |
|---|---|---|---|---|---|---|---|---|---|---|
| | | SEQ | | | | domain | | | | |
| | | ID | N- | αN- | α1 | loop | α2- | | α3- | C- |
| crop | | NO: | terminus | helix | helix | 1 | helix | loop2 | helix | terminus |
| Arabidopsis | CDNA | 11 | 1-246 | 247- | 307- | 340- | 379- | 466- | 487- | 517- |
| thaliana | | | | 291 | 339 | 378 | 465 | 486 | 516 | 534 |
| | protein | 12 | 1-82 | 83- | 103- | 114- | 127- | 156- | 163- | 173- |
| | | | | 97 | 113 | 126 | 155 | 162 | 172 | 178 |
| maize | cDNA | 13 | 1-186 | 187- | 247- | 280- | 319- | 406- | 427- | 457- |
| | | | | 231 | 279 | 318 | 405 | 426 | 456 | 471 |
| | protein | 14 | 1-62 | 63- | 83- | 94- | 107- | 136- | 143- | 153- |
| | | | | 77 | 93 | 106 | 135 | 142 | 152 | 157 |
| rape seed | CDNA | 15 | 1-252 | 253- | 313- | 346- | 385- | 472- | 493- | 523- |
| | | | | 297 | 345 | 384 | 471 | 492 | 522 | 540 |
| | protein | 16 | 1-84 | 85- | 105- | 116- | 129- | 158- | 165- | 175- |
| | | | | 99 | 115 | 128 | 157 | 164 | 174 | 180 |
| sorghum | CDNA | 17 | 1-186 | 187- | 247- | 280- | 319- | 406- | 427- | 457- |
| | | | | 231 | 279 | 318 | 405 | 426 | 456 | 471 |
| | protein | 18 | 1-62 | 63- | 83- | 94- | 107- | 136- | 143- | 153- |
| | | | | 77 | 93 | 106 | 135 | 142 | 152 | 157 |

In certain embodiments, the mutated CENH3 protein or the CENH3 protein conferring or enhancing haploid inducing activity or capability comprises one or more mutated amino acids, preferably one or more amino acid substitutions in the N-terminal domain corresponding to amino acids 1 to 82 of Arabidopsis thaliana CENH3.

In certain embodiments, the mutated CENH3 protein or the CENH3 protein conferring or enhancing haploid inducing activity or capability comprises one or more mutated amino acids, preferably one or more amino acid substitutions in the αN-helix corresponding to amino acids 83 to 97 of Arabidopsis thaliana CENH3.

In certain embodiments, the mutated CENH3 protein or the CENH3 protein conferring or enhancing haploid inducing activity or capability comprises one or more mutated amino acids, preferably one or more amino acid substitutions in the α1-helix to amino acids 103 to 113 of Arabidopsis thaliana CENH3.

In certain embodiments, the mutated CENH3 protein or the CENH3 protein conferring or enhancing haploid inducing activity or capability comprises one or more mutated amino acids, preferably one or more amino acid substitutions in the loop 1 domain to amino acids 114 to 126 of Arabidopsis thaliana CENH3.

In certain embodiments, the mutated CENH3 protein or the CENH3 protein conferring or enhancing haploid inducing activity or capability comprises one or more mutated amino acids, preferably one or more amino acid substitutions in the α2-helix to amino acids 127 to 155 of Arabidopsis thaliana CENH3.

In certain embodiments, the mutated CENH3 protein or the CENH3 protein conferring or enhancing haploid inducing activity or capability comprises one or more mutated amino acids, preferably one or more amino acid substitutions in the loop 2 domain to amino acids 156 to 162 of Arabidopsis thaliana CENH3.

In certain embodiments, the mutated CENH3 protein or the CENH3 protein conferring or enhancing haploid inducing activity or capability comprises one or more mutated amino acids, preferably one or more amino acid substitutions in the α3-helix to amino acids 163 to 172 of Arabidopsis thaliana CENH3.

In certain embodiments, the mutated CENH3 protein or the CENH3 protein conferring or enhancing haploid inducing activity or capability comprises one or more mutated amino acids, preferably one or more amino acid substitutions in the C-terminal domain of CENH3 to amino acids 173 to 178 of Arabidopsis thaliana CENH3.

Preferably wild type Arabidopsis thaliana CENH3 has an amino acid sequence which is at least 90%, preferably at least 95%, more preferably at least 98% identical to a sequence as set forth in SEQ ID NO: 12.

In certain embodiments, the mutated CENH3 protein or the CENH3 protein conferring or enhancing haploid inducing activity or capability comprises one or more mutated amino acids, preferably one or more amino acid substitutions in the N-terminal domain corresponding to amino acids 1 to 62 of Zea mays CENH3.

In certain embodiments, the mutated CENH3 protein or the CENH3 protein conferring or enhancing haploid inducing activity or capability comprises one or more mutated amino acids, preferably one or more amino acid substitutions in the αN-helix corresponding to amino acids 63 to 77 of Zea mays CENH3.

In certain embodiments, the mutated CENH3 protein or the CENH3 protein conferring or enhancing haploid inducing activity or capability comprises one or more mutated amino acids, preferably one or more amino acid substitutions in the α1-helix to amino acids 83 to 93 of Zea mays CENH3.

In certain embodiments, the mutated CENH3 protein or the CENH3 protein conferring or enhancing haploid inducing activity or capability comprises one or more mutated amino acids, preferably one or more amino acid substitutions in the loop 1 domain to amino acids 94 to 106 of Zea mays CENH3.

In certain embodiments, the mutated CENH3 protein or the CENH3 protein conferring or enhancing haploid inducing activity or capability comprises one or more mutated amino acids, preferably one or more amino acid substitutions in the α2-helix to amino acids 107 to 135 of Zea mays CENH3.

In certain embodiments, the mutated CENH3 protein or the CENH3 protein conferring or enhancing haploid inducing activity or capability comprises one or more mutated amino acids, preferably one or more amino acid substitutions in the loop 2 domain to amino acids 136 to 142 of Zea mays CENH3.

In certain embodiments, the mutated CENH3 protein or the CENH3 protein conferring or enhancing haploid inducing activity or capability comprises one or more mutated amino acids, preferably one or more amino acid substitutions in the α3-helix to amino acids 143 to 152 of Zea mays CENH3.

In certain embodiments, the mutated CENH3 protein or the CENH3 protein conferring or enhancing haploid inducing activity or capability comprises one or more mutated amino acids, preferably one or more amino acid substitutions in the C-terminal domain of CENH3 to amino acids 153 to 157 of Zea mays CENH3.

Preferably wild type Zea mays CENH3 has an amino acid sequence which is at least 90%, preferably at least 95%, more preferably at least 98% identical to a sequence as set forth in SEQ ID NO: 14.

In certain embodiments, the mutated CENH3 protein or the CENH3 protein conferring or enhancing haploid inducing activity or capability comprises one or more mutated amino acids, preferably one or more amino acid substitutions in the N-terminal domain corresponding to amino acids 1 to 62 of Sorghum bicolor CENH3.

In certain embodiments, the mutated CENH3 protein or the CENH3 protein conferring or enhancing haploid inducing activity or capability comprises one or more mutated amino acids, preferably one or more amino acid substitutions in the αN-helix corresponding to amino acids 63 to 77 of Sorghum bicolor CENH3.

In certain embodiments, the mutated CENH3 protein or the CENH3 protein conferring or enhancing haploid inducing activity or capability comprises one or more mutated amino acids, preferably one or more amino acid substitutions in the α1-helix to amino acids 83 to 93 of Sorghum bicolor CENH3.

In certain embodiments, the mutated CENH3 protein or the CENH3 protein conferring or enhancing haploid inducing activity or capability comprises one or more mutated amino acids, preferably one or more amino acid substitutions in the loop 1 domain to amino acids 94 to 106 of Sorghum bicolor CENH3.

In certain embodiments, the mutated CENH3 protein or the CENH3 protein conferring or enhancing haploid inducing activity or capability comprises one or more mutated amino acids, preferably one or more amino acid substitutions in the α2-helix to amino acids 107 to 135 of Sorghum bicolor CENH3.

In certain embodiments, the mutated CENH3 protein or the CENH3 protein conferring or enhancing haploid inducing activity or capability comprises one or more mutated amino acids, preferably one or more amino acid substitutions in the loop 2 domain to amino acids 136 to 142 of Sorghum bicolor CENH3.

In certain embodiments, the mutated CENH3 protein or the CENH3 protein conferring or enhancing haploid inducing activity or capability comprises one or more mutated amino acids, preferably one or more amino acid substitutions in the α3-helix to amino acids 143 to 152 of Sorghum bicolor CENH3, the C-terminal domain of CENH3 to amino acids 153 to 157 of Sorghum bicolor CENH3.

Preferably wild type Sorghum bicolor CENH3 has an amino acid sequence which is at least 90%, preferably at least 95%, more preferably at least 98% identical to a sequence as set forth in SEQ ID NO: 18.

In certain embodiments, the mutated CENH3 protein or the CENH3 protein conferring or enhancing haploid inducing activity or capability comprises one or more mutated amino acids, preferably one or more amino acid substitutions in the N-terminal domain corresponding to amino acids 1 to 84 of Brassica napus CENH3.

In certain embodiments, the mutated CENH3 protein or the CENH3 protein conferring or enhancing haploid inducing activity or capability comprises one or more mutated amino acids, preferably one or more amino acid substitutions in the αN-helix corresponding to amino acids 85 to 99 of Brassica napus CENH3.

In certain embodiments, the mutated CENH3 protein or the CENH3 protein conferring or enhancing haploid inducing activity or capability comprises one or more mutated amino acids, preferably one or more amino acid substitutions in the α1-helix to amino acids 105 to 115 of Brassica napus CENH3.

In certain embodiments, the mutated CENH3 protein or the CENH3 protein conferring or enhancing haploid inducing activity or capability comprises one or more mutated amino acids, preferably one or more amino acid substitutions in the loop 1 domain to amino acids 116 to 128 of Brassica napus CENH3.

In certain embodiments, the mutated CENH3 protein or the CENH3 protein conferring or enhancing haploid inducing activity or capability comprises one or more mutated amino acids, preferably one or more amino acid substitutions in the α2-helix to amino acids 129 to 157 of Brassica napus CENH3.

In certain embodiments, the mutated CENH3 protein or the CENH3 protein conferring or enhancing haploid inducing activity or capability comprises one or more mutated amino acids, preferably one or more amino acid substitutions in the loop 2 domain to amino acids 158 to 164 of Brassica napus CENH3.

In certain embodiments, the mutated CENH3 protein or the CENH3 protein conferring or enhancing haploid inducing activity or capability comprises one or more mutated amino acids, preferably one or more amino acid substitutions in the α3-helix to amino acids 165 to 174 of Brassica napus CENH3.

In certain embodiments, the mutated CENH3 protein or the CENH3 protein conferring or enhancing haploid inducing activity or capability comprises one or more mutated amino acids, preferably one or more amino acid substitutions in the C-terminal domain of CENH3 to amino acids 175 to 180 of Brassica napus CENH3.

Preferably wild type Brassica napus CENH3 has an amino acid sequence which is at least 90%, preferably at least 95%, more preferably at least 98% identical to a sequence as set forth in SEQ ID NO: 16.

In certain embodiments, the mutated CENH3 protein or the CENH3 protein conferring or enhancing haploid inducing activity or capability comprises one or more mutated amino acids, preferably one or more amino acid substitutions, in one or more of the N-terminal domain, the αN-helix, the α1-helix, the loop 1 domain, the α2-helix, the loop 2 domain, the α3-helix, or the C-terminal domain of CENH3, such as specified in Table 2.

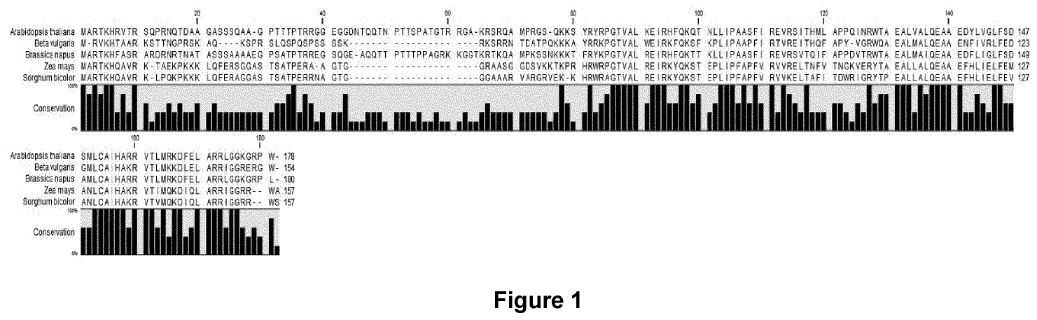

TABLE 2
CENH3 protein mutants validated and positively tested for maternal
haploid induction in maize, rapeseed, Sorghum and Arabidopsis (At)
(see also FIG. 1)

| | | | | | | Pat/mat |
|---|---|---|---|---|---|---|
| | | | | | | (%) |
| | | corresponding | | Mat | Pat | (Haploid = |
| Crop | Mutant | position in At | Domain | % | % | inducer) |
| maize | R3Q | R3 | N- | 0.2 | 0 | 0 |
| | | | terminus | | | |
| maize | P16L | T17 | N- | 0.2 | 0 | 0 |
| | | | terminus | | | |
| maize | A32T | T32 | N- | 0.38 | 0 | 0 |
| | | | terminus | | | |
| maize | E35K | T35 | N- | 0.38 | 0 | 0 |
| | | | terminus | | | |
| maize | A84V | A104 | α1-helix | 0.2 | 0 | 0 |
| maize | P85S | S105 | α1-helix | 0.2 | 0 | 0 |
| maize | V89M | E109 | α1-helix | 0.2 | 0 | 0.3 |
| maize | G100E | P120 | loop 1 | 1.5 | 0 | 0 |
| maize | A128T | S148 | α2-helix | 0.2 | 0 | 0 |
| maize | R155H | G175 | C- | 0.2 | 0 | 0 |
| | | | terminus | | | |
| rape seed | S9F | T9 | N- | 1.6 | 0 | 0 |
| | | | terminus | | | |
| rape seed | S24L | S24 | N- | 0.5 | 0 | 0.5 |
| | | | terminus | | | |
| rape seed | E29K | — | N- | 0.5 | 0 | 0 |
| | | | terminus | | | |
| rape seed | G30D | G29 | N- | 0.5 | 0 | 0 |
| | | | terminus | | | |
| rape seed | A33T | T32 | N- | 0.5 | 0 | 0 |
| | | | terminus | | | |
| rape seed | S41N | E40 | N- | 1.1 | 0 | 0 |
| | | | terminus | | | |
| rape seed | G43E | G42 | N- | 1.1 | 0 | 0.5 |
| | | | terminus | | | |
| rape seed | P50S | P50 | N- | 1.1 | 0 | 0 |
| | | | terminus | | | |
| rape seed | P55L | A55 | N- | 0.5 | 0 | 0.5 |
| | | | terminus | | | |
| rape seed | G57D | G57 | N- | 0.5 | 0 | 0 |
| | | | terminus | | | |
| rape seed | G61E | G61 | N- | 2.2 | 0 | 0 |
| | | | terminus | | | |
| rape seed | L132F | L130 | α2-helix | 0 | 0 | 2.2 |
| rape seed | C153Y | C151 | α2-helix | 0.5 | 0 | 0 |
| rape seed | R159H | R157 | loop2 | 1.1 | 0 | 0 |
| rape seed | V160I | V158 | loop2 | 1.6 | 0 | 0 |
| rape seed | D166N | D164 | α3-helix | 0.5 | 0 | 0 |
| rape seed | E168K | E166 | α3-helix | 1.1 | 0 | 0 |
| Sorghum | G42E | G42 | N- | 0.39 | 0 | 0 |
| | | | terminus | | | |
| Sorghum | E55K | Q74 | N- | 0 | 0 | 0.63 |
| | | | terminus | | | |
| Sorghum | L110F | L130 | α2-helix | 0.07 | 0 | 0 |
| Sorghum | S157L | — | C- | 0.28 | 0 | 0 |
| | | | terminus | | | |
| Arabidopsis | P82S | P82 | N- | | | |
| | | | terminus | | | |
| Arabidopsis | P82L | P82 | N- | | | |
| | | | terminus | | | |
| Arabidopsis | G83E | G83 | αN- | | | |
| | | | helix | | | |
| Arabidopsis | A86T | A86 | αN- | | | |
| | | | helix | | | |
| Arabidopsis | R124C | R124 | loop 1 | | | |
| Arabidopsis | A127V | A127 | α2-helix | | | |
| Arabidopsis | A132V | A132 | α2-helix | | | |
| Arabidopsis | A136V | A136 | α2-helix | | | |
| Arabidopsis | A136T | A136 | α2-helix | | | |
| Arabidopsis | C151Y | C151 | α2-helix | | | |
| Arabidopsis | A152V | A152 | α2-helix | | | |
| Arabidopsis | A155T | A155 | α2-helix | | | |
| Arabidopsis | G172R | G172 | α3-helix | | | |

In certain embodiments, the mutated CENH3 protein or the CENH3 protein conferring or enhancing haploid inducing activity or capability comprises one or more mutated amino acids, preferably one or more amino acid substitutions, as disclosed in WO 2016/030019, WO 2016/102665, or WO 2016/138021 (each of which are incorporated herein by reference in their entirety), or the corresponding mutations in CENH3 orthologues.

In certain embodiments, the mutated CENH3 protein or the CENH3 protein conferring or enhancing haploid inducing activity or capability comprises one or more mutated amino acids, preferably one or more amino acid substitutions, corresponding to positions 3, 17, 32, 35, 9, 24, 29, 40, 42, 50, 55, 57, 61, 74, 82, 104, 109, 120, 148, 175, 130, 151, 157, 158, 164, 166, 83, 86, 124, 127, 132, 136, 152, 155 or 172 of reference Arabidopsis thaliana CENH3 protein, preferably wherein said Arabidopsis thaliana CENH3 protein has an amino acid sequence which is at least 90%, preferably at least 95%, more preferably at least 98% identical to a sequence as set forth in SEQ ID NO: 12.

In certain embodiments, the mutated CENH3 protein or the CENH3 protein conferring or enhancing haploid inducing activity or capability comprises one or more mutated amino acids, preferably one or more amino acid substitutions, corresponding to positions 3, 17, 32, 35, 104, 109, 120, 148 or 175 of Arabidopsis thaliana CENH3 protein if the plant or plant part comprising such sequence is from the genus Zea, preferably Zea mays, preferably wherein said Arabidopsis thaliana CENH3 protein has an amino acid sequence which is at least 90%, preferably at least 95%, more preferably at least 98% identical to a sequence as set forth in SEQ ID NO: 12.

In certain embodiments, the mutated CENH3 protein or the CENH3 protein conferring or enhancing haploid inducing activity or capability comprises one or more mutated amino acids, preferably one or more amino acid substitutions, at positions 3, 16, 32 35, 84, 89, 100, 128 or 155 of CENH3 protein of a plant or plant part from the genus Zea, preferably Zea mays, preferably wherein said Zea mays CENH3 protein has an amino acid sequence which is at least 90%, preferably at least 95%, more preferably at least 98% identical to a sequence as set forth in SEQ ID NO: 14.

In certain embodiments, the mutated CENH3 protein or the CENH3 protein conferring or enhancing haploid inducing activity or capability comprises one or more mutated amino acids, preferably one or more amino acid substitutions, corresponding to positions 9, 24, 29, 32, 40, 42, 50, 55, 57, 61, 130, 151, 157, 158, 164 or 166 of reference Arabidopsis thaliana CENH3 protein if the plant or plant part comprising such sequence is from the genus Brassica, preferably Brassica napus, preferably wherein said Arabidopsis thaliana CENH3 protein has an amino acid sequence which is at least 90%, preferably at least 95%, more preferably at least 98% identical to a sequence as set forth in SEQ ID NO: 12.

In certain embodiments, the mutated CENH3 protein or the CENH3 protein conferring or enhancing haploid inducing activity or capability comprises one or more mutated amino acids, preferably one or more amino acid substitutions, at positions 9, 24, 29, 30, 33, 41, 43, 50, 55, 57, 61, 132, 153, 159, 160, 166 or 168 of CENH3 protein of a plant or plant part from the genus Brassica, preferably Brassica napus, preferably wherein said Brassica napus CENH3 protein has an amino acid sequence which is at least 90%, preferably at least 95%, more preferably at least 98% identical to a sequence as set forth in SEQ ID NO: 16.

In certain embodiments, the mutated CENH3 protein or the CENH3 protein conferring or enhancing haploid inducing activity or capability comprises one or more mutated amino acids, preferably one or more amino acid substitutions, corresponding to positions 42, 74, or 130 of reference Arabidopsis thaliana CENH3 protein if the plant or plant part comprising such sequence is from the genus Sorghum, preferably Sorghum bicolor, preferably wherein said Arabidopsis thaliana CENH3 protein has an amino acid sequence which is at least 90%, preferably at least 95%, more preferably at least 98% identical to a sequence as set forth in SEQ ID NO: 12.

In certain embodiments, the mutated CENH3 protein or the CENH3 protein conferring or enhancing haploid inducing activity or capability comprises one or more mutated amino acids, preferably one or more amino acid substitutions, at positions 42, 55, 110, or 157 of CENH3 protein of a plant or plant part from the genus Sorghum, preferably Sorghum bicolor, preferably wherein said Sorghum bicolor CENH3 protein has an amino acid sequence which is at least 90%, preferably at least 95%, more preferably at least 98% identical to a sequence as set forth in SEQ ID NO: 18.

In a preferred embodiment, the mutated CENH3 protein or the CENH3 protein conferring or enhancing haploid inducing activity or capability comprises an amino acid substitution corresponding to position 35 of Zea mays CENH3, preferably an amino acid substitution corresponding to position 35 of SEQ ID NO: 14 or at position 35 of SEQ ID NO: 14, preferably wherein said amino acid substitution is 35K, such as E35K in Zea mays. Such sequence is preferably comprised in a plant from the genus Zea, preferably Zea mays.

In a preferred embodiment, the mutated CENH3 protein or the CENH3 protein conferring or enhancing haploid inducing activity or capability comprises an amino acid substitution corresponding to position 35 of Sorghum bicolor CENH3, preferably an amino acid substitution corresponding to position 35 of SEQ ID NO: 18 or at position 35 of SEQ ID NO: 18, preferably wherein said amino acid substitution is 35K, such as E35K in Sorghum bicolor. Such sequence is preferably comprised in a plant from the genus Sorghum, preferably Sorghum bicolor.

In a preferred embodiment, the mutated CENH3 protein or the CENH3 protein conferring or enhancing haploid inducing activity or capability comprises an amino acid substitution corresponding to position 36 of Brassica napus CENH3, preferably an amino acid substitution corresponding to position 36 of SEQ ID NO: 16 or at position 36 of SEQ ID NO: 16, preferably wherein said amino acid substitution is 35K, such as T35K in Brassica napus. Such sequence is preferably comprised in a plant from the genus Brassica, preferably Brassica napus.

The skilled person will understand how to determine the corresponding position in CENH3 orthologues.

In a preferred embodiment, the mutated CENH3 protein or the CENH3 protein conferring or enhancing haploid inducing activity or capability comprises an amino acid sequence as set forth in SEQ ID NO: 20. In a preferred embodiment, the mutated CENH3 protein or the CENH3 protein conferring or enhancing haploid inducing activity or capability comprises an amino acid sequence as set forth in SEQ ID NO: 20, an amino acid sequence corresponding to an amino acid sequence as set forth in SEQ ID NO: 20, or an amino acid sequence which is at least 80%, such as at least 90%, preferably at least 95%, more preferably at least 98% identical to a sequence as set forth in SEQ ID NO: 20, and which comprises an amino acid at position 35 or a corresponding amino acid position which is not E. In a preferred embodiment, the mutated CENH3 protein or the CENH3 protein conferring or enhancing haploid inducing activity or capability comprises an amino acid sequence as set forth in SEQ ID NO: 20, an amino acid sequence corresponding to an amino acid sequence as set forth in SEQ ID NO: 20, or an amino acid sequence which is at least 80%, such as at least 90%, preferably at least 95%, more preferably at least 98% identical to a sequence as set forth in SEQ ID NO: 20, and which comprises an amino acid at position 35 or a corresponding amino acid position (such as amino acid position 36 in some species, including Brassica napus) which is K. The skilled person will be able to determine corresponding amino acid positions, such as by suitable alignment algorithms, such as described herein elsewhere.

In an embodiment, the invention relates to a Zea mays plant or plant part (such as pollen or seed) comprising a polynucleic acid encoding a mutated ig1 protein having a sequence as set forth in SEQ ID NO: 1, 2, or 3 or a polynucleic acid encoding a protein having a sequence as set forth is SEQ ID NO: 4 or 5 and comprising a polynucleic acid encoding a CENH3 protein having a sequence as set forth in SEQ ID NO: 20.

In an embodiment, the invention relates to a Zea mays plant or plant part (such as pollen or seed) comprising a polynucleic acid encoding a mutated ig1 protein having a sequence as set forth in SEQ ID NO: 1 a polynucleic acid encoding a protein having a sequence as set forth is SEQ ID NO: 4 or 5 and comprising a polynucleic acid encoding a CENH3 protein having a sequence as set forth in SEQ ID NO: 20.

In an embodiment, the invention relates to a Zea mays plant or plant part (such as pollen or seed) comprising a polynucleic acid encoding a mutated ig1 protein having a sequence as set forth in SEQ ID NO: 2 or a polynucleic acid encoding a protein having a sequence as set forth is SEQ ID NO: 4 and comprising a polynucleic acid encoding a CENH3 protein having a sequence as set forth in SEQ ID NO: 20.

In an embodiment, the invention relates to a Zea mays plant or plant part (such as pollen or seed) comprising a polynucleic acid encoding a mutated ig1 protein having a sequence as set forth in SEQ ID NO: 3 or a polynucleic acid encoding a protein having a sequence as set forth is SEQ ID NO: 5 and comprising a polynucleic acid encoding a CENH3 protein having a sequence as set forth in SEQ ID NO: 20.

In an embodiment, the invention relates to a Zea mays plant or plant part (such as pollen or seed) comprising a polynucleic acid encoding a mutated ig1 protein having a sequence as set forth in SEQ ID NO: 1, 2, or 3 or a polynucleic acid encoding a protein having a sequence as set forth is SEQ ID NO: 4 or 5 and comprising a polynucleic acid encoding a CENH3 protein having an amino acid at position 35 which is different than E, preferably wherein said amino acid is K.

In an embodiment, the invention relates to a Zea mays plant or plant part (such as pollen or seed) comprising a polynucleic acid encoding a mutated ig1 protein having a sequence as set forth in SEQ ID NO: 1 a polynucleic acid encoding a protein having a sequence as set forth is SEQ ID NO: 4 or 5 and comprising a polynucleic acid encoding a CENH3 protein having an amino acid at position 35 which is different than E, preferably wherein said amino acid is K.

In an embodiment, the invention relates to a Zea mays plant or plant part (such as pollen or seed) comprising a polynucleic acid encoding a mutated ig1 protein having a sequence as set forth in SEQ ID NO: 2 or a polynucleic acid encoding a protein having a sequence as set forth is SEQ ID NO: 4 and comprising a polynucleic acid encoding a CENH3 protein having an amino acid at position 35 which is different than E, preferably wherein said amino acid is K.

In an embodiment, the invention relates to a Zea mays plant or plant part (such as pollen or seed) comprising a polynucleic acid encoding a mutated ig1 protein having a sequence as set forth in SEQ ID NO: 3 or a polynucleic acid encoding a protein having a sequence as set forth is SEQ ID NO: 5 and comprising a polynucleic acid encoding a CENH3 protein having an amino acid at position 35 which is different than E, preferably wherein said amino acid is K.

In certain embodiments, the plant or plant part according to the invention as described herein further comprises a site-directed DNA or RNA binding protein or a polynucleic acid encoding a site-directed DNA or RNA binding protein, preferably a site-directed DNA or RNA editing or modification protein. Accordingly, in certain embodiments, the plant or plant part according to the invention as described herein further comprises a site-directed DNA or RNA binding protein or a polynucleic acid encoding a site-directed DNA or RNA editing or modification protein. Such plants as well as methods for producing such plants are for instance described in U.S. Pat. No. 10,285,348, which is incorporated herein by reference in its entirety.

As used herein, the term “site-directed DNA or RNA binding protein” refers to a protein which binds DNA or RNA in a sequence-specific manner or which is recruited to DNA or RNA in a sequence-specific manner, either directly (such as in the case of TALENS or zinc finger nucleases) or indirectly (such as in the case of CRISPR/Cas systems, in which the Cas effector protein binds a DNA or RNA hybridizing guide RNA (comprising a guide sequence and a direct repeat sequence), and optionally (if needed) a tracr sequence). The site-directed DNA or RNA binding protein may edit or modify DNA or RNA directly (i.e. the DNA or RNA binding protein may intrinsically possess the capacity to edit or modify DNA or RNA, such as a Cas effector protein) or may be fused to another protein or domain which has the capacity to edit or modify DNA or RNA (such as is the case for TALENs or ZFNs which are respectively comprise TALEs or ZFs fused to FokI). As used herein, the term “site-directed DNA or RNA editing or modification protein” commonly refers to proteins which either directly or indirectly bind DNA or RNA in a sequence-specific manner and which edit or modify DNA or RNA either directly or indirectly (such as via a fusion partner, i.e. chimeric proteins), and can alternatively be called “editing machinery”.

In certain embodiments, the site-directed DNA or RNA binding protein or DNA or RNA site-directed editing or modification protein is a nuclease (i.e. a DNA or RNA nuclease). In certain embodiments, the site-directed DNA or RNA binding protein or DNA or RNA site-directed editing or modification protein is an endonuclease (i.e. a DNA or RNA endonuclease).

In certain embodiments, the site-directed DNA or RNA binding protein or DNA or RNA site-directed editing or modification protein is a mutated nuclease (i.e. a DNA or RNA nuclease). In certain embodiments, the site-directed DNA or RNA binding protein or DNA or RNA site-directed editing or modification protein is a mutated endonuclease (i.e. a DNA or RNA endonuclease). Such mutated (endo)nuclease may comprise mutations which alter DNA or RNA binding specificity (for instance to alter PAM specificity in case of Cas effector proteins), stability (such as destabilizing mutants), and/or activity (such as mutants enhancing or (partially) abolishing enzymatic activity, for instance catalytically inactive Cas effector proteins or nickase Cas effector proteins). An advantage of catalytically inactive mutants is that they may serve as a vehicle to recruit fusion partners in a sequence-specific manner. Such fusion partners may possess different DNA or RNA editing or modification activities, or even other activities, such as transcription activation or repression activities, chromatin remodelling activity.

In certain embodiments, the site-directed DNA or RNA binding protein or DNA or RNA site-directed editing or modification protein is selected from the group comprising meganucleases (MNs), zinc-finger nucleases (ZFNs), transcription-activator like effector nucleases (TALENs), (mutated) Cas nucleases/effector proteins, such as Cas9, Cfp1 (Cas12a), MAD7, Cas13 (e.g. Cas13a or Cas13b), dCas9-FokI (“dead” or catalytically inactive Cas9 fused to FokI), dCpf1-FokI (“dead” or catalytically inactive Cpf1 fused to FokI), dMAD7-FokI (“dead” or catalytically inactive MAD7 fused to FokI), a nickase Cas effector protein (e.g. Cas9 or Cpf1), chimeric Cas effector (such as Cas9, Cpf1, Cas13)-cytidine deaminase (wherein the Cas effector protein is catalytically inactive), chimeric Cas effector (such as Cas9, Cpf1, Cas13)-adenine deaminase (wherein the Cas effector protein is catalytically inactive), chimeric FENI-FokI, and Mega-TALs, chimeric dCas9 non-FokI nuclease, dCpf1 non-FokI nuclease and dMAD7 non-FokI nuclease. Fusion proteins of for instance Cas effectors (such as Cas9, Cas12, or Cas13) with deaminases, such as adenine or cytidine deaminases allow for base editing, in particular the introduction of point mutations.

As described herein elsewhere, if the site-directed DNA or RNA binding protein is a (mutated) Cas effector protein, sequence specific DNA or RNA binding requires the presence of a guide RNA (gRNA), which hybridizes to a specific target sequence and recruits the Cas effector protein to this target sequence. The gRNA typically comprises a guide sequence (which hybridizes with the target sequence) and a direct repeat (or tracr mate) sequence (which binds to and recruits the Cas effector protein). Depending on the type of Cas effector protein, a tracr sequence may or may not be required, as is known in the art. The gRNA and tracr sequences may be provided on the same or different polynucleic acids. Also chimeric gRNAs (i.e. a fusion of gRNA and tracr) are within the scope of the present invention. The skilled person will understand that the gRNA (and tracr, if needed) can also be comprised in the haploid inducer plant according to the present invention, or can also be expressed in the haploid inducer plant according to the present invention. However, such need not necessarily intrinsically be the case. For instance, only a Cas effector protein can be comprised or expressed in the haploid inducer plant according to the present invention, whereas the appropriate gRNA (and tracr RNA, if required) can be provided (e.g. inserted, transformed, etc) at a separate time.

Plants or plant parts according to the invention, in particular the haploid inducer plants as described herein, such as the paternal haploid inducer plants as described herein which further comprise a site-directed DNA or RNA binding, editing, or modifying protein or a polynucleic acid encoding a site-directed DNA or RNA binding, editing, or modifying protein as described herein allow for simultaneous haploid induction and gene editing. The editing machinery is delivered via the inducer line. The editing machinery is encoded by and are present in the inducer line because they have been stably inserted in the inducer, for example, via bombardment or agrobacterium mediated transformation. In other examples, the editing machinery is transiently introduced (through exogenous application) or transiently expressed in the gametophyte prior to fertilization.

After fertilization, edits are made by the editing machinery in the non-inducer target genes prior to or during elimination of the inducer chromosomes. The result is a haploid embryo or plant or seed that contains the chromosome set only from the non-inducer parent, where that chromosome set contains DNA sequences that have been edited. These edited haploids can be identified, grown, and their chromosomes doubled, preferably by colchicine, pronamide, dithipyr, trifluralin, or another known anti-microtubule agent. This line can then be directly used in downstream breeding programs.

In certain embodiments, the editing machinery is any DNA modification enzyme, but is preferably a site-directed nuclease. The site-directed nuclease is preferably CRISPR-based, but could also be a meganuclease, a transcription-activator like effector nuclease (TALEN), or a zinc finger nuclease. The nuclease used in this invention could be Cas9, Cfp1, dCas9-FokI, chimeric FEN1-FokI. In one aspect, the DNA modification enzyme is a site-directed base editing enzyme such as Cas9 (or Cpf1, etc.)—cytidine deaminase fusion protein or Cas9 (or Cpf1, etc.)—adenine deaminase fusion protein, wherein the Cas9 (or Cpf1, etc.) can have one or both of its nuclease activity inactivated, i.e. chimeric Cas9 (or Cpf1, etc.) nickase (nCas9, nCpf1, etc.) or deactivated Cas9 (dCas9, dCpf1, etc.) fused to cytidine deaminase or adenine deaminase. The optional guide RNA targets the genome at the specific site intended to be edited.

In an aspect, the invention relates to a plant or plant part obtained or obtainable from crossing a first plant, which is a plant according to the invention as described herein, with a second plant. In an aspect, the invention relates to a plant or plant part obtained or obtainable from crossing a first female plant, which is a plant according to the invention as described herein, with a second male plant. In an aspect, the invention relates to a plant or plant part obtained or obtainable from pollinating a second plant by pollen from a first plant, which is a plant according to the invention as described herein.

In an aspect, the invention relates to a method for generating a plant or plant part, comprising crossing a first plant, which is a plant according to the invention as described herein, with a second plant. In an aspect, the invention relates to a method for generating a plant or plant part, comprising crossing a first female plant, which is a plant according to the invention as described herein, with a second male plant. In an aspect, the invention relates to a method for generating a plant or plant part, comprising pollinating a second plant with pollen from a first plant which is a plant according to the invention as described herein.

In an aspect, the invention relates to Zea mays seed designated igEIN, a representative sample of which has been deposited under NCIMB (National Collection of Industrial Food and Marine Bacteria; Ltd. Ferguson Building, Craibstone Estate, Bucksburn, Aberdeen, AB21 9YA Scotland) on May 11, 2021, Accession No. NCIMB 43772, or plants or plant parts grown or obtained therefrom. In an aspect, the invention relates to Zea mays seed as deposited under NCIMB Accession No. NCIMB 43772, or plants or plant parts grown or obtained therefrom. Plants grown or obtained from seed deposited under NCIMB Accession No. NCIMB 43772 exhibit a (increased) haploid inducer phenotype (on average). Seed deposited under NCIMB Accession No. NCIMB 43772 comprises a CENH3 mutation resulting in an E35K amino acid exchange (SEQ ID NO: 20) and comprise an ig nucleotide sequence as set forth in SEQ ID NO: 1, as described in Example 1.

In an aspect, the invention relates to a method for generating a haploid plant or plant part, comprising crossing a first plant, which is a plant according to the invention as described herein, with a second plant, and selecting a haploid progeny plant or plant part. In an aspect, the invention relates to a method for generating a haploid plant or plant part, comprising crossing a first female plant, which is a plant according to the invention as described herein, with a second male plant, and selecting a haploid progeny plant or plant part. In an aspect, the invention relates to a method for generating a haploid plant or plant part, comprising pollinating a second plant with pollen from a first plant which is a plant according to the invention as described herein, and selecting a haploid progeny plant or plant part. It will be understood that haploid progeny includes dihapolid, trihaploid, etc. progeny, as described herein elsewhere. Optionally, the method further comprises generating a doubled haploid plant or plant part from said haploid plant or plant part or converting said haploid plant or plant part into a doubled haploid plant or plant part.

In an aspect, the invention relates to a method for generating a plant or plant part, comprising providing a haploid plant or plant part obtained or obtainable from crossing a first plant, which is a plant according to the invention as described herein, with a second plant, and converting the haploid plant or plant part into a doubled haploid plant or plant part. In an aspect, the invention relates to a method for generating a plant or plant part, comprising providing a haploid plant or plant part obtained or obtainable from crossing a first female plant, which is a plant according to the invention as described herein, with a second male plant, and converting the haploid plant or plant part into a doubled haploid plant or plant part. In an aspect, the invention relates to a method for generating a plant or plant part, comprising providing a haploid plant or plant part obtained or obtainable from pollinating a second plant by pollen from a first plant, which is a plant according to the invention as described herein, and converting the haploid plant or plant part into a doubled haploid plant or plant part. It will be understood that haploid plant or plant part includes dihapolid, trihaploid, etc. plant or plant part, as described herein elsewhere.

In an aspect, the invention relates to a method for generating a (doubled haploid) plant or plant part, comprising crossing a first plant, which is a plant according to the invention as described herein, with a second plant, and converting haploid progeny into a doubled haploid plant or plant part. In an aspect, the invention relates to a method for generating a (doubled haploid) plant or plant part, comprising crossing a first female plant, which is a plant according to the invention as described herein, with a second male plant, and converting haploid progeny into a doubled haploid plant or plant part. In an aspect, the invention relates to a method for generating a (doubled haploid) plant or plant part, comprising pollinating a second plant with pollen from a first plant which is a plant according to the invention as described herein, and converting haploid progeny into a doubled haploid plant or plant part.

In an aspect, the invention provides a method of editing a plant's genomic DNA. This is done by taking a first plant—which is a haploid inducing plant and which also has encoded into its DNA the machinery necessary for accomplishing the editing (for example, a Cas9 enzyme and a guide RNA)—and using that first plant's pollen to pollinate a second plant. The second plant is the plant to be edited. From that pollination event, progeny (e.g., embryos or seeds) are produced; at least one of which will be a haploid seed. This haploid seed will only contain the chromosomes of the second plant; the first plant's chromosomes have vanished (having been eliminated, lost or degraded), but before doing so, the first plant's chromosomes permitted the gene-editing machinery to be expressed, or the first plant delivers the already-expressed editing machinery upon pollination via the pollen tube. Or, in the case that the haploid inducer line is the female in the cross, the haploid inducing plant's egg cell contains the editing machinery that is present and perhaps already being expressed, upon fertilization with the “wild type” or non-haploid inducing pollen grain. Through any of these routes, the haploid progeny obtained by the cross will also have had its genome edited.

One embodiment of the invention provides a method of editing plant genomic DNA, comprising: (i) providing a first plant, wherein the first plant is a haploid inducer line of the plant according to the invention as described herein, and wherein said first plant comprises, expresses, or is capable of expressing a DNA modification enzyme as described herein elsewhere, and optionally a guide RNA; (ii) providing a second plant, wherein the second plant comprises the plant genomic DNA which is to be edited; (iii) crossing the first and second plant, or pollinating the second plant with pollen from the first plant; and (iv) selecting at least one haploid progeny produced by the pollination of step (c) wherein the haploid progeny comprises the genome of the second plant but not the first plant, and the genome of the haploid progeny has been modified by the DNA modification enzyme and optional guide nucleic acid delivered by the first plant.

In an aspect, the invention relates to a method of editing or modifying plant genomic DNA or RNA, comprising: a) providing a first plant which is a plant according to the invention as described herein and comprising, expressing, or capable of expressing a site-directed DNA or RNA binding protein as described herein elsewhere; b) providing a second plant (comprising the plant genomic DNA or RNA which is to be modified); c) pollinating the second maize plant with pollen from the first plant; and d) selecting at least one haploid progeny produced by the pollination of step c) (wherein the haploid, dihaploid or trihaploid progeny comprises the genome of the second plant but not the first plant, and the genome of the haploid, dihaploid or trihaploid progeny has been modified by the site-directed DNA or RNA binding protein delivered by the first plant).

The methods of the invention as described herein may further comprise the step of harvesting plant material, such as preferably seeds (resulting from the cross or pollination).

The methods of the invention as described herein may further comprise the step of selecting haploid progeny resulting from the cross or pollination. It will be understood that haploid progeny includes dihapolid, trihaploid, etc. progeny, as described herein elsewhere.

The methods of the invention as described herein may further comprise the step of crossing the progeny, preferably backcrossing the progeny (resulting from the cross or pollination). The methods of the invention as described herein may further comprise the step of selfing the progeny (resulting from the cross or pollination).

The methods of the invention as described herein may further comprise the step of regenerating a plant or plant part (from the embryo resulting from the cross or pollination).

The methods of the invention as described herein may further comprise the step of converting a haploid plant or plant part (resulting from the cross or pollination) into a doubled haploid plant or plant part. It will be understood that haploid progeny includes dihapolid, trihaploid, etc. progeny, as described herein elsewhere. Methods for generating doubled haploid plants are known in the art and are described herein elsewhere.

Preferably, the second plant is not a plant according to the present invention. Preferably, the second plant is not a haploid inducing plant.

Preferably, the second plant is from the same species as the first plant. In certain embodiments, the first and second plant are from the genus Zea, preferably Zea mays. In certain embodiments, the first and second plant are from the genus Sorghum, preferably Sorghum bicolor. In certain embodiments, the first and second plant are from the genus Brassica, preferably Brassica napus.

In an aspect, the invention relates to a progeny plant or plant part obtained or obtainable by the methods according to the invention as described herein.

It will be understood that the polynucleic acid encoding a mutated indeterminate gametophyte (ig) protein or haploid inducing or enhancing ig protein and the polynucleic acid encoding a mutated centromere or kinetochore protein or haploid inducing or enhancing centromere or kinetochore protein are operatively linked to one or more regulatory sequences in a plant or plant part, in particular a promoter sequence, thereby allowing expression of the protein. Such promoter may be the endogenous promoter or may be an exogenous (heterologous) promoter. Such promoter may be in its native genomic location or may not be in its native genomic location. Such promoter may allow for constitutive, transient, or conditional expression, such as expression depending on developmental level, tissue-specific expression, inducible expression, etc. The same holds true for the site-directed DNA or RNA binding protein encoding polynucleic acids as described herein elsewhere.

The term “regulatory sequence” as used herein relates to a nucleotide sequence which affects the specificity and/or the expression strength, e.g., in that the regulatory sequence mediates a defined tissue specificity. Such a regulatory sequence may be located upstream of the transcription initiation point of a minimal promoter, but also downstream of it, e.g., as in a transcribed, but untranslated, leader sequence or within an intron.

In certain embodiments, the polynucleic acid sequences according to the invention as described herein can be introduced in a plant or plant part by transformation, such as Agrobacterium tumefaciens mediated transformation, as is known in the art. Hereto, the polynucleic acid may be provided on a suitable vector.

As used herein, a “vector” has its ordinary meaning in the art, and may for instance be a plasmid, a cosmid, a phage or an expression vector, a transformation vector, shuttle vector, or cloning vector; it may be double- or single-stranded, linear or circular; or it may transform a prokaryotic or eukaryotic host, either via integration into its genome or extrachromosomally. The nucleic acid according to the invention is preferably operatively linked in a vector with one or more regulatory sequences which allow the transcription, and, optionally, the expression, in a prokaryotic or eukaryotic host cell. A regulatory sequence—preferably, DNA—may be homologous or heterologous to the nucleic acid according to the invention. For example, the nucleic acid is under the control of a suitable promoter or terminator. Suitable promoters may be promoters which are constitutively induced (example: 35S promoter from the “Cauliflower mosaic virus” (Odell et al., 1985); those promoters which are tissue-specific are especially suitable (example: Pollen-specific promoters, Chen et al. (2010), Zhao et al. (2006), or Twell et al. (1991)), or are development-specific (example: blossom-specific promoters). Suitable promoters may also be synthetic or chimeric promoters which do not occur in nature, are composed of multiple elements, and contain a minimal promoter, as well as—upstream of the minimum promoter—at least one cis-regulatory element which serves as a binding location for special transcription factors. Chimeric promoters may be designed according to the desired specifics and are induced or repressed via different factors. Examples of such promoters are found in Gurr & Rushton (2005) or Venter (2007). For example, a suitable terminator is the nos-terminator (Depicker et al., 1982). The vector may be introduced via conjugation, mobilization, biolistic transformation, agrobacteria-mediated transformation, transfection, transduction, vacuum infiltration, or electroporation.

In certain embodiments, the vector is a conditional expression vector. In certain embodiments, the vector is a constitutive expression vector. In certain embodiments, the vector is a tissue-specific expression vector, such as a pollen-specific expression vector. In certain embodiments, the vector is an inducible expression vector. All such vectors are well-known in the art.

Methods for preparation of the described vectors are commonplace to the person skilled in the art (Sambrook et al., 2001).

Also envisaged herein is a host cell, such as a plant cell, which comprises a nucleic acid as described herein, preferably an induction-promoting nucleic acid or a nucleic acid encoding a double-stranded RNA as described herein, or a vector as described herein. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

The host cell may be a prokaryotic (for example, bacterial) or eukaryotic cell (for example, a plant cell or a yeast cell). For example, the host cell may be an agrobacterium, such as Agrobacterium tumefaciens or Agrobacterium rhizogenes. Preferably, the host cell is a plant cell.

A nucleic acid described herein or a vector described herein may be introduced in a host cell via well-known methods, which may depend on the selected host cell, including, for example, conjugation, mobilization, biolistic transformation, agrobacteria-mediated transformation, transfection, transduction, vacuum infiltration, or electroporation. In particular, methods for introducing a nucleic acid or a vector in an agrobacterium cell are well-known to the skilled person and may include conjugation or electroporation methods. Also methods for introducing a nucleic acid or a vector into a plant cell are known (Sambrook et al., 2001) and may include diverse transformation methods such as biolistic transformation and agrobacterium-mediated transformation.

In particular embodiments, the present invention relates to a transgenic plant cell which comprises a nucleic acid as described herein, in particular an induction-promoting nucleic acid or a nucleic acid encoding a double-stranded RNA as described herein, as a transgene or a vector as described herein. In further embodiments, the present invention relates to a transgenic plant or a part thereof which comprises the transgenic plant cell.

For example, such a transgenic plant cell or transgenic plant is a plant cell or plant which is, preferably stably, transformed with a nucleic acid as described herein, in particular an induction-promoting nucleic acid or a nucleic acid encoding a double-stranded RNA as described herein, or a vector as described herein.

Preferably, the nucleic acid in the transgenic plant cell is operatively linked with one or more regulatory sequences which allow the transcription, and optionally the expression, in the plant cell. A regulatory sequence may be homologous or heterologous to the nucleic acid. The total structure made up of the nucleic acid according to the invention and the regulatory sequence(s) may then represent the transgene.

A part of a transgenic plant may be, for example, a fertilized or unfertilized seed, an embryo, a pollen, a tissue, an organ, or a plant cell, wherein the fertilized or unfertilized seed, the embryo, or the pollen are generated in the transgenic plant, and the nucleic acid as described herein, in particular an induction-promoting nucleic acid or a nucleic acid encoding a double-stranded RNA as described herein, is integrated into its genome as a transgene or the vector. The term transgenic plant as used herein also includes a descendant of the transgenic plant described herein in whose genome the nucleic acid as described herein, in particular an induction-promoting nucleic acid or a nucleic acid encoding a double-stranded RNA as described herein, is integrated as a transgene or the vector.

As used herein, the term “operatively linked” or “operably linked” means connected in a common nucleic acid molecule in such a manner that the connected elements are positioned and oriented relative to one another such that a transcription of the nucleic acid molecule may occur. A DNA which is operatively linked with a promoter is under the transcriptional control of this promoter.

As used herein the term “transformation” refers to the transfer of isolated and cloned genes into the DNA, usually the chromosomal DNA or genome, of another organism.

As used herein, the term “sequence identity” refers to the degree of identity between any given nucleic acid sequence and a target nucleic acid sequence. As used herein, unless explicitly specified, sequence identity is preferably determined over the entire sequence length. Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (Bl2seq) program from the stand-alone version of BLASTZ containing BLASTN and BLASTP. This stand-alone version of BLASTZ can be obtained from Fish & Richardson's web site (World Wide Web at fr.com/blast) or the U.S. government's National Center for Biotechnology Information web site (World Wide Web at ncbi.nlm.nih.gov). Instructions explaining how to use the Bl2seq program can be found in the readme file accompanying BLASTZ. Bl2seq performs a comparison between two sequences using either the BLASTN or BLASTP algorithm.

BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq I .txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. The following command will generate an output file containing a comparison between two sequences: C:\B12seq-i c:\seqI.txt-j c:\seg2.txt-p blastn-o c:\output.txt-q-1-r 2. If the target sequence shares homology with any portion of the identified sequence, then the designated output file will present those regions of homology as aligned sequences. If the target sequence does not share homology with any portion of the identified sequence, then the designated output file will not present aligned sequences. Once aligned, a length is determined by counting the number of consecutive nucleotides from the target sequence presented in alignment with the sequence from the identified sequence starting with any matched position and ending with any other matched position. A matched position is any position where an identical nucleotide is presented in both the target and identified sequences. Gaps presented in the target sequence are not counted since gaps are not nucleotides. Likewise, gaps presented in the identified sequence are not counted since target sequence nucleotides are counted, not nucleotides from the identified sequence. The percent identity over a particular length is determined by counting the number of matched positions over that length and dividing that number by the length followed by multiplying the resulting value by 100. For example, if (i) a 500-base nucleic acid target sequence is compared to a subject nucleic acid sequence, (ii) the Bl2seq program presents 200 bases from the target sequence aligned with a region of the subject sequence where the first and last bases of that 200-base region are matches, and (iii) the number of matches over those 200 aligned bases is 180, then the 500-base nucleic acid target sequence contains a length of 200 and a sequence identity over that length of 90% (i.e., 180/200×100=90). It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

An “isolated nucleic acid sequence” or “isolated DNA” refers to a nucleic acid sequence which is no longer in the natural environment from which it was isolated, e.g. the nucleic acid sequence in a bacterial host cell or in the plant nuclear or plastid genome. When referring to a “sequence” herein, it is understood that the molecule having such a sequence is referred to, e.g. the nucleic acid molecule. A “host cell” or a “recombinant host cell” or “transformed cell” are terms referring to a new individual cell (or organism) arising as a result of at least one nucleic acid molecule, having been introduced into said cell. The host cell is preferably a plant cell or a bacterial cell. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

In certain embodiments, the nucleic acid molecule as described herein comprises less than 50000 nucleotides. In certain embodiments, the nucleic acid molecule as described herein comprises less than 40000 nucleotides. In certain embodiments, the nucleic acid molecule as described herein comprises less than 30000 nucleotides. In certain embodiments, the nucleic acid molecule as described herein comprises less than 25000 nucleotides. In certain embodiments, the nucleic acid molecule as described herein comprises less than 20000 nucleotides. In certain embodiments, the nucleic acid molecule as described herein comprises less than 15000 nucleotides. In certain embodiments, the nucleic acid molecule as described herein comprises less than 10000 nucleotides. In certain embodiments, the nucleic acid molecule as described herein comprises less than 5000 nucleotides. In certain embodiments, the nucleotide molecule as described herein comprises at least 100 nucleotides. In certain embodiments, the nucleic acid molecule as described herein comprises at least 100 nucleotides and less than 50000 nucleotides. In certain embodiments, the nucleic acid molecule as described herein comprises at least 100 nucleotides and less than 40000 nucleotides. In certain embodiments, the nucleic acid molecule as described herein comprises at least 100 nucleotides and less than 30000 nucleotides. In certain embodiments, the nucleic acid molecule as described herein comprises at least 100 nucleotides and less than 25000 nucleotides. In certain embodiments, the nucleic acid molecule as described herein comprises at least 100 nucleotides and less than 20000 nucleotides. In certain embodiments, the nucleic acid molecule as described herein comprises at least 100 nucleotides and less than 15000 nucleotides. In certain embodiments, the nucleic acid molecule as described herein comprises at least 100 nucleotides and less than 10000 nucleotides. In certain embodiments, the nucleic acid molecule as described herein comprises at least 100 nucleotides and less than 5000 nucleotides.

When reference is made to a nucleic acid sequence (e.g. DNA or genomic DNA) having “substantial sequence identity to” a reference sequence or having a sequence identity of at least 80%>, e.g. at least 85%, 90%, 95%, 98%> or 99%> nucleic acid sequence identity to a reference sequence, in one embodiment said nucleotide sequence is considered substantially identical to the given nucleotide sequence and can be identified using stringent hybridisation conditions. In another embodiment, the nucleic acid sequence comprises one or more mutations compared to the given nucleotide sequence but still can be identified using stringent hybridisation conditions. “Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

“RNA interference” or “RNAi” is a biological process in which RNA molecules inhibit gene expression or translation, by neutralizing targeted mRNA molecules. Two types of small ribonucleic acid (RNA) molecules—microRNA (miRNA) and small interfering RNA (siRNA)— are central to RNA interference. RNAs are the direct products of genes, and these small RNAs can bind to other specific messenger RNA (mRNA) molecules and either increase or decrease their activity, for example by preventing an mRNA from being translated into a protein. The RNAi pathway is found in many eukaryotes, including animals, and is initiated by the enzyme Dicer, which cleaves long double-stranded RNA (dsRNA) molecules into short double-stranded fragments of about 21 nucleotide siRNAs (small interfering RNAs). Each siRNA is unwound into two single-stranded RNAs (ssRNAs), the passenger strand and the guide strand. The passenger strand is degraded and the guide strand is incorporated into the RNA-induced silencing complex (RISC). Mature miRNAs are structurally similar to siRNAs produced from exogenous dsRNA, but before reaching maturity, miRNAs must first undergo extensive post-transcriptional modification. A miRNA is expressed from a much longer RNA-coding gene as a primary transcript known as a pri-miRNA which is processed, in the cell nucleus, to a 70-nucleotide stem-loop structure called a pre-miRNA by the microprocessor complex. This complex consists of an RNase III enzyme called Drosha and a dsRNA-binding protein DGCR8. The dsRNA portion of this pre-miRNA is bound and cleaved by Dicer to produce the mature miRNA molecule that can be integrated into the RISC complex; thus, miRNA and siRNA share the same downstream cellular machinery. A short hairpin RNA or small hairpin RNA (shRNA/Hairpin Vector) is an artificial RNA molecule with a tight hairpin turn that can be used to silence target gene expression via RNA interference. The most well-studied outcome is post-transcriptional gene silencing, which occurs when the guide strand pairs with a complementary sequence in a messenger RNA molecule and induces cleavage by Argonaute 2 (Ago2), the catalytic component of the RISC. In will be understood that the RNAi molecules can be applied as such to/in the plant, or can be encoded by appropriate vectors, from which the RNAi molecule is expressed. Delivery and expression systems of RNAi molecules, such as siRNAs, shRNAs or miRNAs are well known in the art.

Mutations as described herein may be introduced by mutagenesis, which may be performed in accordance with any of the techniques known in the art. As used herein, “mutagenization” or “mutagenesis” includes both conventional mutagenesis and location-specific mutagenesis or “genome editing” or “gene editing”. In conventional mutagenesis, modification at the DNA level is not produced in a targeted manner. The plant cell or the plant is exposed to mutagenic conditions, such as TILLING, via UV light exposure or the use of chemical substances (Till et al., 2004). An additional method of random mutagenesis is mutagenesis with the aid of a transposon. Location-specific mutagenesis enables the introduction of modification at the DNA level in a target-oriented manner at predefined locations in the DNA. For example, TALENS, meganucleases, homing endonucleases, zinc finger nucleases, or a CRISPR/Cas system as further described herein may be used for this.

Mutations as described herein may be introduced by random mutagenesis. The skilled person will understand that identification and selection of suitable mutations may include appropriate selection assays, such as functional selection assays (including genotypic or phenotypic selection assays). In random mutagenesis, cells or organisms may be exposed to mutagens such as UV, X-ray, or gamma ray radiation or mutagenic chemicals (such as for instance such as ethyl methanesulfonate (EMS), ethylnitrosourea (ENU), or dimethylsulfate (DMS), and mutants with desired characteristics are then selected. Mutants can for instance be identified by TILLING (Targeting Induced Local Lesions in Genomes). The method combines mutagenesis, such as mutagenesis using a chemical mutagen such as ethyl methanesulfonate (EMS) with a sensitive DNA screening-technique that identifies single base mutations/point mutations in a target gene. The TILLING method relies on the formation of DNA heteroduplexes that are formed when multiple alleles are amplified by PCR and are then heated and slowly cooled. A “bubble” forms at the mismatch of the two DNA strands, which is then cleaved by a single stranded nuclease. The products are then separated by size, such as by HPLC. See also McCallum et al. “Targeted screening for induced mutations”; Nat Biotechnol. 2000 April; 18(4):455-7 and McCallum et al. “Targeting Induced Local Lesions IN Genomes (TILLING) for plant functional genomics”; Plant Physiol. 2000 June; 123(2):439-42, both incorporated by reference in their entirety. By means of further example, and without limitation, the methodologies described in the following publications, incorporated by reference in their entirety, may be adopted according to the present invention, such as in connection with EMS mutagenesis: Till et al. “Discovery of induced point mutations in maize genes by TILLING”; BMC Plant Biol. 2004 Jul. 28; 4:12; and Weil & Monde “Getting the point-mutations in maize” Crop Sci 2007; 47 S60-S67. The skilled person will understand that depending on the mutagen dose (irradiation of chemical) the (average) mutation density can be varied or fixed. In certain embodiments, the random mutagenesis is single nucleotide mutagenesis. In certain embodiments, the random mutagenesis is chemical mutagenesis, preferably EMS mutagenesis.

“Gene editing” or “genome editing” or “gene modification” or “genome modification” refers to genetic engineering in which in which DNA or RNA is inserted, deleted, modified or replaced in the genome (or transcriptome) of a living organism. Accordingly, gene editing encompassed DNA editing and RNA editing. Gene editing may comprise targeted or non-targeted (random) mutagenesis. Targeted mutagenesis may be accomplished for instance with designer nucleases, such as for instance with meganucleases, zinc finger nucleases (ZFNs), transcription activator-like effector-based nucleases (TALEN), and the clustered regularly interspaced short palindromic repeats (CRISPR/Cas9) system. These nucleases create site-specific double-strand breaks (DSBs) at desired locations in the genome. The induced double-strand breaks are repaired through nonhomologous end-joining (NHEJ) or homologous recombination (HR), resulting in targeted mutations or nucleic acid modifications. The use of designer nucleases is particularly suitable for generating gene knockouts or knockdowns. In certain embodiments, designer nucleases are developed which specifically induce a mutation in the ig and/or centromere or kinetochore gene, as described herein elsewhere, such as to generate a mutation or a knockout of the gene. Alternatively, by means of for instance RNA-specific CRISPR/Cas systems, a knockdown can be achieved, as RNA/specific CRISPR/Cas systems (such as Cas13) allow site-directed cleavage of (single-stranded) RNA. Accordingly, in certain embodiments, designer nucleases, in particular RNA-specific CRISPR/Cas systems are developed which specifically target the mRNA, such as to cleave mRNA and generate a knockdown of the gene/mRNA/protein. Delivery and expression systems of designer nuclease systems are well known in the art.

In certain embodiments, the nuclease or targeted/site-specific/homing nuclease is, comprises, consists essentially of, or consists of a (modified) CRISPR/Cas system or complex, a (modified) Cas protein, a (modified) zinc finger, a (modified) zinc finger nuclease (ZFN), a (modified) transcription factor-like effector (TALE), a (modified) transcription factor-like effector nuclease (TALEN), or a (modified) meganuclease. In certain embodiments, said (modified) nuclease or targeted/site-specific/homing nuclease is, comprises, consists essentially of, or consists of a (modified) RNA-guided nuclease. It will be understood that in certain embodiments, the nucleases may be codon optimized for expression in plants. As used herein, the term “targeting” of a selected nucleic acid sequence means that a nuclease or nuclease complex is acting in a nucleotide sequence specific manner. For instance, in the context of the CRISPR/Cas system, the guide RNA is capable of hybridizing with a selected nucleic acid sequence. As used herein, “hybridization” or “hybridizing” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. Hybridization is a process in which a single-stranded nucleic acid molecule attaches itself to a complementary nucleic acid strand, i.e. agrees with this base pairing. Standard procedures for hybridization are described, for example, in Sambrook et al. (Molecular Cloning. A Laboratory Manual, Cold Spring Harbor Laboratory Press, 3rd edition 2001). Preferably this will be understood to mean an at least 50%, more preferably at least 55%, 60%, 65%, 70%, 75%, 80% or 85%, more preferably 90%, 91%, 92%, 93%, 94%, 95%, 96%, 97%, 98% or 99% of the bases of the nucleic acid strand form base pairs with the complementary nucleic acid strand. A hybridization reaction may constitute a step in a more extensive process, such as the initiation of PGR, or the cleavage of a polynucleotide by an enzyme. A sequence capable of hybridizing with a given sequence is referred to as the “complement” of the given sequence.

Gene editing may involve transient, inducible, or constitutive expression of the gene editing components or systems. Gene editing may involve genomic integration or episomal presence of the gene editing components or systems. Gene editing components or systems may be provided on vectors, such as plasmids, which may be delivered by appropriate delivery vehicles, as is known in the art. Preferred vectors are expression vectors.

Gene editing may comprise the provision of recombination templates, to effect homology directed repair (HDR). For instance a genetic element may be replaced by gene editing in which a recombination template is provided. The DNA may be cut upstream and downstream of a sequence which needs to be replaced. As such, the sequence to be replaced is excised from the DNA. Through HDR, the excised sequence is then replaced by the template.

In certain embodiments, the nucleic acid modification or mutation is effected by a (modified) transcription activator-like effector nuclease (TA LEN) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference. By means of further guidance, and without limitation, naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26. The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

In certain embodiments, the nucleic acid modification or mutation is effected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference. By means of further guidance, and without limitation, artificial zinc-finger (ZF) technology involves arrays of ZF modules to target new DNA-binding sites in the genome. Each finger module in a ZF array targets three DNA bases. A customized array of individual zinc finger domains is assembled into a ZF protein (ZFP). ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms.

In certain embodiments, the nucleic acid modification is effected by a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, the nucleic acid modification is effected by a (modified) CRISPR/Cas complex or system. With respect to general information on CRISPR/Cas Systems, components thereof, and delivery of such components, including methods, materials, delivery vehicles, vectors, particles, and making and using thereof, including as to amounts and formulations, as well as Cas9CRISPR/Cas-expressing eukaryotic cells, Cas-9 CRISPR/Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809), WO 2015/089351 (PCT/US2014/069897), WO 2015/089354 (PCT/US2014/069902), WO 2015/089364 (PCT/US2014/069925), WO 2015/089427 (PCT/US2014/070068), WO 2015/089462 (PCT/US2014/070127), WO 2015/089419 (PCT/US2014/070057), WO 2015/089465 (PCT/US2014/070135), WO 2015/089486 (PCT/US2014/070175), PCT/US2015/051691, PCT/US2015/051830. Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/835,973, 61/836,080, 61/836,101, and 61/836,127, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT/US2014/62558 filed Oct. 28, 2014, and US Provisional Patent Applications Ser. Nos. 61/915,148, 61/915,150, 61/915,153, 61/915,203, 61/915,251, 61/915,301, 61/915,267, 61/915,260, and 61/915,397, each filed Dec. 12, 2013; 61/757,972 and 61/768,959, filed on Jan. 29, 2013 and Feb. 25, 2013; 62/010,888 and 62/010,879, both filed Jun. 11, 2014; 62/010,329, 62/010,439 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/055,484, 62/055,460 and 62/055,487, each filed Sep. 25, 2014; and 62/069,243, filed Oct. 27, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to PCT application designating, inter alia, the United States, application No. PCT/US14/41806, filed Jun. 10, 2014. Mention is also made of U.S. application 62/180,709, 17-Jun.-15, PROTECTED GUIDE RNAS (PGRNAS); U.S. application 62/091,455, filed, 12-Dec.-14, PROTECTED GUIDE RNAS (PGRNAS); U.S. application 62/096,708, 24-Dec.-14, PROTECTED GUIDE RNAS (PGRNAS); U.S. applications 62/091,462, 12-Dec.-14, 62/096,324, 23-Dec.-14, 62/180,681, 17 Jun. 2015, and 62/237,496, 5 Oct. 2015, DEAD GUIDES FOR CRISPR TRANSCRIPTION FACTORS; U.S. application 62/091,456, 12-Dec.-14 and 62/180,692, 17 Jun. 2015, ESCORTED AND FUNCTIONALIZED GUIDES FOR CRISPR-CAS SYSTEMS; U.S. application 62/091,461, 12-Dec.-14, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19-Dec.-14, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24-Dec.-14, ENGINEERING OF SYSTEMS, METHODS AND OPTIMIZED ENZYME AND GUIDE SCAFFOLDS FOR SEQUENCE MANIPULATION; U.S. application 62/098,059, 30-Dec.-14, 62/181,641, 18 Jun. 2015, and 62/181,667, 18 Jun. 2015, RNA-TARGETING SYSTEM; U.S. application 62/096,656, 24-Dec.-14 and 62/181,151, 17 Jun. 2015, CRISPR HAVING OR ASSOCIATED WITH DESTABILIZATION DOMAINS; U.S. application 62/096,697, 24-Dec.-14, CRISPR HAVING OR ASSOCIATED WITH AAV; U.S. application 62/098,158, 30-Dec.-14, ENGINEERED CRISPR COMPLEX INSERTIONAL TARGETING SYSTEMS; U.S. application 62/151,052, 22-Apr.-15, CELLULAR TARGETING FOR EXTRACELLULAR EXOSOMAL REPORTING; U.S. application 62/054,490, 24-Sep.-14, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12-F

EB-14, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25-Sep.-14, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4-Dec.-14, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24-Sep.-14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23-Oct.-14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24-Sep.-14 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24-Sep.-14, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25-Sep.-14, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25-Sep.-14, MULTIFUNCTIONAL-CRISPR COMPLEXES AND/OR OPTIMIZED ENZYME LINKED FUNCTIONAL-CRISPR COMPLEXES; U.S. application 62/087,475, 4-Dec.-14 and 62/181,690, 18 Jun. 2015, FUNCTIONAL SCREENING WITH OPTIMIZED FUNCTIONAL CRISPR-CAS SYSTEMS; U.S. application 62/055,487, 25-Sep.-14, FUNCTIONAL SCREENING WITH OPTIMIZED FUNCTIONAL CRISPR-CAS SYSTEMS; U.S. application 62/087,546, 4-Dec.-14 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30-Dec.-14, CRISPR MEDIATED IN VIVO MODELING AND GENETIC SCREENING OF TUMOR GROWTH AND METASTASIS. Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, and Attorney Docket No. 46783.01.2128, filed 22 Oct. 2015, NOVEL CRISPR ENZYMES AND SYSTEMS, U.S. application 62/232,067, 24 Sep. 2015, U.S. application 62/205,733, 16 Aug. 2015, U.S. application 62/201,542, August 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12-Dec.-2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES. European patent application EP3009511. Reference is further made to Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013); RNA-guided editing of bacterial genomes using CRISPR-Cas systems. Jiang W., Bikard D., Cox D., Zhang F, Marraffini L A. Nat Biotechnol March; 31(3):233-9 (2013); One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013); Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. 2013 Aug. 22; 500(7463):472-6. doi: 10.1038/Nature12466. Epub 2013 Aug. 23; Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5. (2013); DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013); Genome engineering using the CRISPR-Cas9 system. Ran, F A., Hsu, P D., Wright, J., Agarwala, V., Scott, D A., Zhang, F. Nature Protocols November; 8(11):2281-308. (2013); Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print]; Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27. (2014). 156(5):935-49; Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. (2014) Apr. 20. doi: 10.1038/nbt.2889; CRISPR-Cas9 Knockin Mice for Genome Editing and Cancer Modeling, Platt et al., Cell 159(2): 440-455 (2014) DOI: 10.1016/j.cell.2014.09.014; Development and Applications of CRISPR-Cas9 for Genome Engineering, Hsu et al, Cell 157, 1262-1278 (Jun. 5, 2014) (Hsu 2014); Genetic screens in human cells using the CRISPR/Cas9 system, Wang et al., Science. 2014 Jan. 3; 343(6166): 80-84. doi:10.1126/science.1246981; Rational design of highly active sgRNAs for CRISPR-Cas9-mediated gene inactivation, Doench et al., Nature Biotechnology 32(12):1262-7 (2014) published online 3 Sep. 2014; doi:10.1038/nbt.3026, and In vivo interrogation of gene function in the mammalian brain using CRISPR-Cas9, Swiech et al, Nature Biotechnology 33, 102-106 (2015) published online 19 Oct. 2014; doi:10.1038/nbt.3055, Cpf1 Is a Single RNA-Guided Endonuclease of a Class 2 CRISPR-Cas System, Zetsche et al., Cell 163, 1-13 (2015); Discovery and Functional Characterization of Diverse Class 2 CRISPR-Cas Systems, Shmakov et al., Mol Cell 60(3): 385-397 (2015); C2c2 is a single-component programmable RNA-guided RNA-targeting CRISPR effector, Abudayyeh et al, Science (2016) published online Jun. 2, 2016 doi: 10.1126/science.aaf5573. Each of these publications, patents, patent publications, and applications, and all documents cited therein or during their prosecution (“appin cited documents”) and all documents cited or referenced in the appin cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appin cited documents) are incorporated herein by reference to the same extent as if each individual document was specifically and individually indicated to be incorporated by reference.

In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide.

In general, the CRISPR/Cas or CRISPR system is as used herein foregoing documents refers collectively to transcripts and other elements involved in the expression of or directing the activity of CRISPR-associated (“Cas”) genes, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cpf1).

As used herein, the term “crRNA” or “guide RNA” or “single guide RNA” or “sgRNA” or “one or more nucleic acid components” of a CRISPR/Cas locus effector protein, as applicable, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay.

A guide sequence, and hence a nucleic acid-targeting guide RNA may be selected to target any target nucleic acid sequence. The target sequence may be DNA. The target sequence may be genomic DNA. The target sequence may be mitochondrial DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In certain embodiments, the gRNA comprises a stem loop, preferably a single stem loop. In certain embodiments, the direct repeat sequence forms a stem loop, preferably a single stem loop. In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In particular embodiments, the CRISPR/Cas system requires a tracrRNA. The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and gRNA sequence are contained within a single transcript, such that hybridization between the two produces a transcript having a secondary structure, such as a hairpin. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In a hairpin structure the portion of the sequence 5′ of the final “N” and upstream of the loop may correspond to the tracr mate sequence, and the portion of the sequence 3′ of the loop then corresponds to the tracr sequence. In a hairpin structure the portion of the sequence 5′ of the final “N” and upstream of the loop may alternatively correspond to the tracr sequence, and the portion of the sequence 3′ of the loop corresponds to the tracr mate sequence. In alternative embodiments, the CRISPR/Cas system does not require a tracrRNA, as is known by the skilled person.

In certain embodiments, the guide RNA (capable of guiding Cas to a target locus) may comprise (1) a guide sequence capable of hybridizing to a target locus and (2) a tracr mate or direct repeat sequence (in 5′ to 3′ orientation, or alternatively in 3′ to 5′ orientation, depending on the type of Cas protein, as is known by the skilled person). In particular embodiments, the CRISPR/Cas protein is characterized in that it makes use of a guide RNA comprising a guide sequence capable of hybridizing to a target locus and a direct repeat sequence, and does not require a tracrRNA. In particular embodiments, where the CRISPR/Cas protein is characterized in that it makes use of a tracrRNA, the guide sequence, tracr mate, and tracr sequence may reside in a single RNA, i.e. an sgRNA (arranged in a 5′ to 3′ orientation or alternatively arranged in a 3′ to 5′ orientation), or the tracr RNA may be a different RNA than the RNA containing the guide and tracr mate sequence. In these embodiments, the tracr hybridizes to the tracr mate sequence and directs the CRISPR/Cas complex to the target sequence.

Typically, in the context of an endogenous nucleic acid-targeting system, formation of a nucleic acid-targeting complex (comprising a guide RNA hybridized to a target sequence and complexed with one or more nucleic acid-targeting effector proteins) results in modification (such as cleavage) of one or both DNA or RNA strands in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g. within 1, 2, 3, 4, 5, 6, 7, 8, 9, 10, 20, 50, or more base pairs from the target sequence, wherein the target sequence is comprised within a target locus of interest). The skilled person will be aware of specific cut sites for selected CRISPR/Cas systems, relative to the target sequence, which as is known in the art may be within the target sequence or alternatively 3′ or 5′ of the target sequence.

In some embodiments, the unmodified nucleic acid-targeting effector protein may have nucleic acid cleavage activity. In some embodiments, the nuclease as described herein may direct cleavage of one or both nucleic acid (DNA, RNA, or hybrids, which may be single or double stranded) strands at the location of or near a target sequence, such as within the target sequence and/or within the complement of the target sequence or at sequences associated with the target sequence. In some embodiments, the nucleic acid-targeting effector protein may direct cleavage of one or both DNA or RNA strands within about 1, 2, 3, 4, 5, 6, 7, 8, 9, 10, 15, 20, 25, 50, 100, 200, 500, or more base pairs from the first or last nucleotide of a target sequence. In some embodiments, the cleavage may be blunt (e.g. for Cas9, such as SaCas9 or SpCas9). In some embodiments, the cleavage may be staggered (e.g. for Cpf1), i.e. generating sticky ends. In some embodiments, the cleavage is a staggered cut with a 5′ overhang. In some embodiments, the cleavage is a staggered cut with a 5′ overhang of 1 to 5 nucleotides, preferably of 4 or 5 nucleotides. In some embodiments, the cleavage site is upstream of the PAM. In some embodiments, the cleavage site is downstream of the PAM. In some embodiments, the nucleic acid-targeting effector protein that may be mutated with respect to a corresponding wild-type enzyme such that the mutated nucleic acid-targeting effector protein lacks the ability to cleave one or both DNA or RNA strands of a target polynucleotide containing a target sequence. As a further example, two or more catalytic domains of a Cas protein (e.g. RuvC I, RuvC II, and RuvC III or the HNH domain of a Cas9 protein) may be mutated to produce a mutated Cas protein substantially lacking all DNA cleavage activity. In some embodiments, a nucleic acid-targeting effector protein may be considered to substantially lack all DNA and/or RNA cleavage activity when the cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form. As used herein, the term “modified” Cas generally refers to a Cas protein having one or more modifications or mutations (including point mutations, truncations, insertions, deletions, chimeras, fusion proteins, etc.) compared to the wild type Cas protein from which it is derived. By derived is meant that the derived enzyme is largely based, in the sense of having a high degree of sequence homology with, a wildtype enzyme, but that it has been mutated (modified) in some way as known in the art or as described herein.

In certain embodiments, the target sequence should be associated with a PAM (protospacer adjacent motif) or PFS (protospacer flanking sequence or site); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). Examples of PAM sequences are given in the examples section below, and the skilled person will be able to identify further PAM sequences for use with a given CRISPR enzyme. Further, engineering of the PAM Interacting (PI) domain may allow programing of PAM specificity, improve target site recognition fidelity, and increase the versatility of the Cas, e.g. Cas9, genome engineering platform. Cas proteins, such as Cas9 proteins may be engineered to alter their PAM specificity, for example as described in Kleinstiver B P et al. Engineered CRISPR-Cas9 nucleases with altered PAM specificities. Nature. 2015 Jul. 23; 523(7561):481-5. doi: 10.1038/nature14592. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence. The skilled person will understand that other Cas proteins may be modified analogously.

The Cas protein as referred to herein, such as without limitation Cas9, Cpf1 (Cas12a), C2c1 (Cas12b), C2c2 (Cas13a), C2c3, Cas13b protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is (modified) Cpf1, preferably Acidaminococcus sp., such as Acidaminococcus sp. BV3L6 Cpf1 (AsCpf1) or Lachnospiraceae bacterium Cpf1, such as Lachnospiraceae bacterium MA2020 or Lachnospiraceae bacterium MD2006 (LbCpf1). In certain embodiments, the Cas protein is (modified) C2c2, preferably Leptotrichia wadei C2c2 (LwC2c2) or Listeria newyorkensis FSL M6-0635 C2c2 (LbFSLC2c2). In certain embodiments, the (modified) Cas protein is C2c1. In certain embodiments, the (modified) Cas protein is C2c3. In certain embodiments, the (modified) Cas protein is Cas13b.

A doubled haploid plant or plant part is one that is developed by the doubling of a haploid set of chromosomes. A plant or seed that is obtained from a doubled haploid plant that is selfed any number of generations may still be identified as a doubled haploid plant. A doubled haploid plant is considered a homozygous plant. A plant is considered to be doubled haploid if it is fertile, even if the entire vegetative part of the plant does not consist of the cells with the doubled set of chromosomes. For example, a plant will be considered a doubled haploid plant if it contains viable gametes, even if it is chimeric.

Somatic haploid cells, haploid embryos, haploid seeds, or haploid seedlings produced from haploid seeds can be treated with a chromosome doubling agent. Homozygous plants can be regenerated from haploid cells by contacting the haploid cells, such as embryo cells or callus produced from such cells, with chromosome doubling agents, such as colchicine, pronamide, dithipyr, trifluralin, or another known anti-microtubule agent or anti-microtubule herbicide, or nitrous oxide to create homozygous doubled haploid cells. Treatment of a haploid seed or the resulting seedling generally produces a chimeric plant, partially haploid and partially doubled haploid. It may be beneficial to nick the seedling before treatment with colchicine. When reproductive tissue contains doubled haploid cells, then doubled haploid seed is produced.

In an aspect, the invention relates to a method for identifying a plant or plant part, such as a plant or plant part according to the invention, such as described herein elsewhere. Accordingly, in an aspect, the invention relates to a method for identifying a plant or plant part having haploid inducing activity or having enhanced haploid inducing activity (such as described herein elsewhere). In an aspect, the invention relates to a method for identifying a plant or plant part comprising or expressing (a polynucleic acid encoding) a mutated indeterminate gametophyte allele, gene, or protein and (a polynucleic acid encoding) a mutated centromere or kinetochore allele, gene, or protein, preferably CENH3 (such as described herein elsewhere). In an aspect, the invention relates to a method for identifying a plant or plant part comprising or expressing (a polynucleic acid encoding) an indeterminate gametophyte allele, gene, or protein conferring or enhancing haploid inducing activity or capability and (a polynucleic acid encoding) a centromere or kinetochore allele, gene, or protein, preferably CENH3, conferring or enhancing haploid inducing activity or capability (such as described herein elsewhere). In an aspect, the invention relates to a method for identifying a plant or plant part having reduced expression, stability, and/or activity of an indeterminate gametophyte allele, gene, or protein and (a polynucleic acid encoding) a mutated centromere or kinetochore allele, gene, or protein, preferably CENH3 (such as described herein elsewhere). In an aspect, the invention relates to a method for identifying a plant or plant part having reduced expression, stability, and/or activity of an indeterminate gametophyte allele, gene, or protein and comprising (a polynucleic acid encoding) a centromere or kinetochore allele, gene, or protein, preferably CENH3, conferring or enhancing haploid inducing activity or capability (such as described herein elsewhere).

In certain embodiments, such method comprises detecting the mutated indeterminate gametophyte allele, gene, or protein and detecting the mutated centromere or kinetochore, preferably CENH3, allele, gene, or protein (such as described herein elsewhere). In certain embodiments, such method comprises detecting the indeterminate gametophyte allele, gene, or protein having haploid inducing activity or having enhanced haploid inducing activity and detecting the centromere or kinetochore, preferably CENH3, allele, gene, or protein having haploid inducing activity or having enhanced haploid inducing activity (such as described herein elsewhere). In certain embodiments, such method comprises detecting the reduced expression, stability, and/or activity of indeterminate gametophyte allele, gene, or protein and detecting the mutated centromere or kinetochore, preferably CENH3, allele, gene, or protein (such as described herein elsewhere). In certain embodiments, such method comprises detecting the reduced expression, stability, and/or activity of indeterminate gametophyte allele, gene, or protein and detecting the centromere or kinetochore, preferably CENH3, allele, gene, or protein having haploid inducing activity or having enhanced haploid inducing activity (such as described herein elsewhere). In certain embodiments, such method comprises providing a sample comprising (genomic) DNA from a plant or plant part. In certain embodiments, such method comprises assaying for the presence of the ig allele, gene, or protein mutation and the centromere or kinetochore allele, gene, or protein mutation or assaying for the haploid inducing or enhancing ig allele, gene, or protein mutation and assaying for the haploid inducing or enhancing centromere or kinetochore allele, gene, or protein mutation. The skilled person will understand that assaying for a mutation can be direct or indirect, i.e. the mutation may be detected directly (by appropriate assays, as described herein elsewhere), or may be detected indirectly for instance by detection of linked or associated (molecular or genetic) markers (as described herein elsewhere).

In an aspect, the invention relates to a method for generating a plant or plant part, comprising mutagenizing one or more (endogenous) ig allele, gene or protein encoding polynucleic acid and one or more (endogenous) centromere or kinetochore protein allele, gene, or protein encoding polynucleic acid, preferably CENH3, and/or introducing one or more mutated ig allele, gene or protein encoding polynucleic acid and one or more mutated centromere or kinetochore protein allele, gene, or protein encoding polynucleic acid, preferably CENH3. The skilled person will understand that a single allele may be mutated and that homozygosity may be achieved in subsequent generations. The skilled person will understand that the ig and centromere or kinetochore protein may be mutated simultaneously or subsequently, in either order. For instance, in a first stage, ig (or a polynucleic acid encoding the ig protein) may be mutated, and in a subsequent stage, which may be in the same plant or plant part or which may be in a plant or plant part of one or more subsequent generation(s), a centromere or kinetochore protein (or polynucleic acid encoding a centromere or kinetochore protein) may be mutated, or vice versa.

Any means of mutagenesis may be applied, as described herein elsewhere, and include for instance random mutagenesis as well as site-directed mutagenesis.

The aspects and embodiments of the invention are further supported by the following non-limiting examples.

TABLE
description of sequences disclosed herein

| SEQ | |
|---|---|
| ID NO | Description |
| 1 | genomic DNA sequence of haploid induction conferring ig allele |
| 2 | cDNA1 of haploid induction conferring ig allele |
| 3 | cDNA2 of haploid induction conferring ig allele |
| 4 | protein 1 of haploid induction conferring ig allele |
| 5 | protein 2 of haploid induction conferring ig allele |
| 6 | genomic DNA of ig gene derived from reference genome B73 |
| 7 | cDNA1 of ig gene derived from reference genome B73 |
| 8 | cDNA2 of ig gene derived from reference genome B73 |
| 9 | protein 1 of ig gene derived from reference genome B73 |
| 10 | protein 2 of ig gene derived from reference genome B73 |
| 11 | nucleotide sequence of the wildtype CENH3 cDNA of A. |
| | thaliana |
| 12 | amino acid sequence of the wildtype CENH3 protein of A. |
| | thaliana |
| 13 | nucleotide sequence of the wildtype CENH3 cDNA of Z. mays |
| 14 | amino acid sequence of the wildtype CENH3 protein of Z. mays |
| 15 | nucleotide sequence of the wildtype CENH3 cDNA of B. napus |
| 16 | amino acid sequence of the wildtype CENH3 protein of B. napus |
| 17 | nucleotide sequence of the wildtype CENH3 cDNA of S. bicolor |
| 18 | amino acid sequence of the wildtype CENH3 protein of S. |
| | bicolor |
| 19 | nucleotide sequence of the mutated CENH3 (E35K) cDNA of |
| | Z. mays |
| 20 | amino acid sequence of the mutated CENH3 protein (E35K) of |
| | Z. mays |
| 21 | genomic DNA of ig gene 1 derived from Sorghum bicolor |
| 22 | cDNA of ig gene 1 derived from Sorghum bicolor |
| 23 | protein encoded by ig gene 1 derived from Sorghum bicolor |
| 24 | genomic DNA of ig gene 2 derived from Sorghum bicolor |
| 25 | cDNA of ig gene 2 derived from Sorghum bicolor |
| 26 | protein encoded by ig gene 2 derived from Sorghum bicolor |
| 27 | genomic DNA of ig gene 1 derived from Brassica napus |
| 28 | cDNA of ig gene 1 derived from Brassica napus |
| 29 | protein encoded by ig gene 1 derived from Brassica napus |
| 30 | genomic DNA of ig gene 2 derived from Brassica napus |
| 31 | cDNA of ig gene 2 derived from Brassica napus |
| 32 | protein encoded by ig gene 2 derived from Brassica napus |
| 33 | nucleotide sequence of the wildtype CENH3 cDNA of B. |
| | vulgaris |
| 34 | amino acid sequence of the wildtype CENH3 protein of B |
| | vulgaris |

### EXAMPLES

### Example 1

A mutation of CenH3 (E35K) which showed low maternal induction on its own in maize was introgressed to ig-Alvey, a maize line possessing a haploid inducer ig-allele (cf. SEQ ID NO: 1). After 4 backcross generations, the genomic background of ig-Alvey was reconstituted to 99%. The major difference consists in the exchange of the CenH3 alleles. This line was tested for maternal and paternal induction using a glossy mutant as tester and marker analysis and flow cytometry for ploidy confirmation. The maternal induction rate was approximately 0.5%. But independent of the backcross version the paternal induction rate increased to an average of 5.7-7.5%, which is much higher than expected from ig-Alvey alone (1-3%).

TABLE 1
Results of paternal haploid induction of different backcross versions
in first induction test. Haploids have been identified by marker and
flow cytometry analyses. Paternal haploid induction rate (pHIR).

| | number of | | |
|---|---|---|---|
| | kernels | number | |
| | analyzed | of | |
| backcross version | for ploidy | haploids | pHIR |
| 5WVm003b160033-BC10.03.10.6.SE11 | 1343 | 88 | 6.6% |
| 5WVm003b160033-BC10.03.10.13.SE18 | 431 | 46 | 10.7% |
| 5WVm003b160033-BC10.03.10.13.SE23 | 280 | 19 | 6.8% |

TABLE 2
Results of paternal haploid induction of different backcross
versions in the second induction test. Haploids have
been identified by marker and flow cytometry analyses.
Paternal haploid induction rate (pHIR).

| | number of | | |
|---|---|---|---|
| | kernels | number | |
| | analyzed | of | |
| backcross version | for ploidy | haploids | pHIR |
| 5WVm003b160033-BC10.03.10.6.SE11.40 | 454 | 29 | 6.4% |
| 5WVm003b160033-BC10.03.10.13.SE18.30 | 429 | 29 | 6.8% |
| 5WVm003b160033-BC10.03.10.13.SE23.4 | 452 | 18 | 4.0% |

TABLE 3
Results of haploid induction of parental lines. Paternal haploid
induction rate (pHIR) and maternal haploid induction rate (mHIR):

| | number of kernels | number of | | |
|---|---|---|---|---|
| Genotype | analyzed for ploidy | haploids | pHIR | mHIR |
| ig-Alvey | 385 | 4 | 1.0% | — |
| CenH3 (E35K) mutant | 533 | 2 | 0% | 0.4% |

True paternal haploids were not found in the induction tests with different mutations in CenH3 gene alone. However, maternal induction rates could be used as an indication that tested mutation has a potential to increase induction rate when combined with another mutation.

1. A plant or plant part comprising a polynucleic acid encoding a mutated indeterminate gametophyte (ig) protein and a polynucleic acid encoding a mutated centromere or kinetochore protein.

2. The plant or plant part according to claim 1, wherein said polynucleic acid encoding said mutated ig protein comprises an insertion of one or more nucleic acids compared to the polynucleic acid encoding the wild-type indeterminate gametophyte (ig) protein, or wherein said polynucleic acid encoding said mutated ig protein comprises a knockout mutation or a knockdown mutation.

3. The plant or plant part according to claim 1, wherein said polynucleic acid encoding said mutated ig protein comprises an insertion of one or more nucleic acids in an ig codon corresponding to a codon selected from codon 118, 119, or 120 of the wild type Zea mays ig protein, such as set forth SEQ ID NO: 7 or 8, corresponding to a codon selected from codon 191, 192, or 193 of the wild type Sorghum bicolor ig protein, such as set forth in SEQ ID NO: 22, corresponding to a codon selected from codon 143, 144, or 145 of the wild type Sorghum bicolor ig protein, such as set forth in SEQ ID NO: 25, or corresponding to a codon selected from codon 94, 95 or 96 of the wild type Brassica napus ig protein, such as set forth in SEQ ID NO: 28 or 31.

4. The plant or plant part according to claim 1, wherein said mutated centromere or kinetochore protein is selected from the group comprising CENH3, CENP-C, KNL2, SCM3, SAD2 and SIM3, preferably CENH3.

5. The plant or plant part according to claim 4, wherein said mutated CENH3 protein comprises one or more mutated amino acids corresponding to positions 3, 17, 32, 35, 9, 24, 29, 40, 42, 50, 55, 57, 61, 74, 82, 104, 109, 120, 148, 175, 130, 151, 157, 158, 164, 166, 83, 86, 124, 127, 132, 136, 152, 155 or 172 of reference Arabidopsis thaliana CENH3 protein, preferably wherein said Arabidopsis thaliana CENH3 protein has an amino acid sequence which is at least 90%, preferably at least 95%, more preferably at least 98% identical to a sequence as set forth in SEQ ID NO: 12.

6. The plant or plant part according to claim 1, wherein said plant or plant part is selected from the group comprising the genera Zea, Sorghum, and Brassica, preferably Zea mays, Sorghum bicolor, and Brassica napus.

7. The plant or plant part according to claim 1, wherein said plant is from the genus Zea, preferably Zea mays, wherein said mutated indeterminate gametophyte (ig) protein
a) is encoded by a polynucleic acid comprising the nucleotide sequence of SEQ ID NO: 1 or a sequence which is at least 90% identical, preferably at least 95% identical, more preferable at least 98% identical to SEQ ID NO: 1;
b) is derived from a coding sequence comprising the nucleotide sequence of SEQ ID NO: 2 or 3, or a sequence which is at least 90% identical, preferably at least 95% identical, more preferable at least 98% identical to SEQ ID NO: 2 or 3; or
c) has an amino acid sequence of SEQ ID NO: 4 or 5, or which is at least 90% identical, preferably at least 95% identical, more preferable at least 98% identical to SEQ ID NO: 4 or 5.

8. The plant or plant part according to claim 1, wherein said plant is Zea mays and wherein said mutated centromere or kinetochore protein is mutated CENH3 protein having an amino acid substitution at position 35, preferably an amino acid substitution corresponding to position 35 of SEQ ID NO: 14 or at position 35 of SEQ ID NO: 14, preferably wherein said amino acid substitution is 35K, such as E35K.

9. The plant according to claim 1, further comprising a polynucleic acid encoding a site-directed DNA or RNA binding protein.

10. The plant according to claim 9, wherein said site-directed DNA or RNA binding protein is a mutated nuclease selected from the group comprising meganucleases (MNs), zinc-finger nucleases (ZFNs), transcription-activator like effector nucleases (TALENs), mutated Cas nucleases/effector proteins, such as Cas9 nuclease, Cfp1 nuclease, MAD7 nuclease, dCas9-FokI, dCpf1-FokI, dMAD7 nuclease-FokI, chimeric Cas9-cytidine deaminase, chimeric Cas9-adenine deaminase, chimeric FENI-FokI, and Mega-TALs, a nickase Cas9 (nCas9), chimeric dCas9 non-FokI nuclease, dCpf1 non-FokI nuclease and dMAD7 non-FokI nuclease.

11. A method for generating a plant or plant part, comprising providing a haploid, dihaploid, or trihaploid plant resulting from crossing a first plant which is a plant according to claim 1 with a second plant and converting the haploid, dihaploid, or trihaploid plant or plant part into a doubled haploid, doubled dihaploid, or doubled trihaploid plant or plant part.

12. A method for generating a plant according to claim 1, comprising the steps of:
A) (i) providing a plant or plant part; and
(ii) mutating one or more endogenous ig allele, gene, or protein encoding polynucleic acid, and mutating one or more endogenous centromere or kinetochore protein allele, gene, or protein encoding polynucleic acid, and/or genomically introducing the one or more mutated ig allele, gene, or protein encoding polynucleic acid, and the one or more mutated centromere or kinetochore protein allele, gene, or protein encoding polynucleic acid; or
B) (i) providing a plant or plant part comprising the one or more endogenous mutated ig allele, gene, or protein encoding polynucleic acid, and/or the one or more genomically introduced mutated ig allele, gene, or protein encoding polynucleic acid; and
(ii) mutating the one or more endogenous centromere or kinetochore protein allele, gene, or protein encoding polynucleic acid, and/or genomically introducing the one or more mutated centromere or kinetochore protein allele, gene, or protein encoding polynucleic acid; or
C) (i) providing a plant or plant part comprising the one or more endogenous mutated centromere or kinetochore protein allele, gene, or protein encoding polynucleic acid, and/or the one or more genomically introduced mutated centromere or kinetochore allele, gene, or protein encoding polynucleic acid; and
(ii) mutating the one or more endogenous ig allele, gene, or protein encoding polynucleic acid, and/or genomically introducing the one or more mutated ig allele, gene, or protein encoding polynucleic acid,
wherein said mutated ig allele, gene, or protein encoding polynucleic acid comprises an insertion of one or more nucleic acids compared to the polynucleic acid encoding the wild-type indeterminate gametophyte (ig) protein, or wherein said polynucleic acid encoding said mutated ig protein comprises a knockout mutation or a knockdown mutation, and
wherein said mutated centromere or kinetochore protein allele, gene, or protein encoding polynucleic acid is selected from the group comprising CENH3, CENP-C, KNL2, SCM3, SAD2 and SIM3, preferably CENH3.

13. A method for identifying a plant or plant part according to claim 1, comprising detecting a mutated indeterminate gametophyte (ig) protein, and a mutated centromere or kinetochore protein, or detecting a polynucleic acid encoding an indeterminate gametophyte protein comprising a mutation, and a polynucleic acid encoding a centromere or kinetochore protein comprising a mutation,
wherein said mutated ig protein or polynucleic acid encoding a mutated ig protein comprises an insertion of one or more nucleic acids compared to the polynucleic acid encoding the wild-type indeterminate gametophyte (ig) protein, or wherein said polynucleic acid encoding said mutated ig protein comprises a knockout mutation or a knockdown mutation, and
wherein said mutated centromere or kinetochore protein or polynucleic acid encoding a mutated centromere or kinetochore protein is selected from the group comprising CENH3, CENP-C, KNL2, SCM3, SAD2 and SIM3, preferably CENH3.

14. A method of modifying plant genomic DNA, comprising: a) providing a first plant which is a plant according to claim 11; b) providing a second plant comprising the plant genomic DNA which is to be modified; c) pollinating the second maize plant with pollen from the first plant; and d) selecting at least one haploid, dihaploid or trihaploid progeny produced by the pollination of step (c) wherein the haploid, dihaploid or trihaploid progeny comprises the genome of the second plant but not the first plant, and the genome of the haploid, dihaploid or trihaploid progeny has been modified by the site-directed DNA or RNA binding protein delivered by the first plant.

15. A method of using a plant or plant part according to claim 1 as a haploid inducer, preferably a paternal haploid inducer.

16. A Zea mays seed as deposited under NCIMB Deposit number NCIMB 43772.

17. A (igEIN) Zea mays seed, a representative sample of which has been deposited under NCIMB Deposit No. NCIMB 43772.

18. A Zea mays plant grown or obtained from the seed according to claim 16.

19. A Zea mays plant part grown or obtained from the seed according to claim 16 or obtained from a plant grown or obtained from the seed according to claim 16.

20. A method for identifying or selecting a plant or plant part, such as a plant or plant part having enhanced haploid inducing activity or capability, comprising:
i) providing a plant or plant part having reduced expression, stability, and/or activity of an indeterminate gametophyte (ig) gene, mRNA, or protein;
ii) mutating a gene encoding a centromere or kinetochore protein, preferably CENH3; and
iii) analysing haploid inducing activity or capability in said plant or plant part, or offspring thereof;
optionally further comprising:
iv) selecting a plant or plant part having enhanced haploid inducing activity or capability.

21. A method for identifying or selecting a plant or plant part, such as a plant or plant part having enhanced haploid inducing activity or capability, comprising:
i) providing a first plant having reduced expression, stability, and/or activity of an indeterminate gametophyte (ig) gene, mRNA, or protein;
ii) crossing said first plant with a second plant having a gene encoding a mutated centromere or kinetochore protein, preferably CENH3; and
iii) analysing haploid inducing activity or capability in the resulting offspring thereof;
optionally further comprising:
iv) selecting a plant or plant part having enhanced haploid inducing activity or capability.

22. A method of using a plant or plant part having reduced expression, stability, and/or activity of an indeterminate gametophyte (ig) gene, mRNA, or protein for screening for or identifying centromere or kinetochore protein, preferably CENH3, mutations conferring or enhancing haploid inducing activity or capability.
